(19) United States
(12) Patent Application Publication
(10) Pub. No.: US 2023/0219892 A1
(43) Pub. Date: Jul. 13, 2023

(54) FORMS AND COMPOSITIONS OF A BETA ADRENERGIC AGONIST

(71) Applicant: Curasen Therapeutics, Inc., San Carlos, CA (US)

(72) Inventors: Wei Chen, San Carlos, CA (US); Bing Xue, San Carlos, CA (US); David Scott Carter, San Carlos, CA (US)

(21) Appl. No.: 18/007,726

(22) PCT Filed: Jun. 3, 2021
(86) PCT No.: PCT/US2021/035796

§ 371 Date: Dec. 1, 2022

Related U.S. Application Data
(60) Provisional application No. 63/034,900, filed on Jun. 4, 2020.

(51) Int. Cl. C07D 213/84; C07C 69/78; C07C 61/06; C07C 59/08; C07C 59/11; C07C 59/245; C07C 59/255; C07C 59/06; C07C 57/15; C07C 57/145; C07C 309/30

(52) U.S. Cl. CPC C07D 213/84; C07C 57/15; C07C 57/145; C07C 59/06; C07C 59/08; C07C 59/11; C07C 59/245; C07C 59/255; C07C 61/06; C07C 69/78; C07C 309/30; C07B 2200/13

(57) ABSTRACT

The present disclosure relates generally to various forms and compositions useful as beta adrenergic agonists and uses of the same in the treatment of diseases associated with an adrenergic receptor. In one aspect, the disclosure provides a crystalline solid form of Compound 1: selected from Form A and Form B and salt forms thereof.

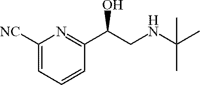

### CROSS-REFERENCE TO RELATED APPLICATION(S)

This application claims the benefit of priority under 35 U.S.C. §119(e) of U.S. Provisional Pat. Application No. 63/034,900, filed Jun. 4, 2020. The disclosure of the prior application is considered part of and is incorporated by reference in the disclosure of this application.

### FIELD OF THE INVENTION

The present disclosure relates generally to various forms and compositions useful as beta adrenergic agonists and uses of the same in the treatment of diseases associated with an adrenergic receptor.

### BACKGROUND

PCT Application Publication Number WO 2017/197324 discloses “[a]drenergic receptor modulating compounds and methods ... of treating a subject for a disease or condition associated with an adrenergic receptor including administering a therapeutically effective amount of the subject compound.”

U.S. Pat. Application Publication Number 2013/0096126 discloses “a method for enhancing learning or memory of both in a mammal having impaired learning or memory or both from a neuro-degenerative disorder, which entails the step of administering at least one compound or a salt thereof which is a β1-adrenergic receptor agonist, partial agonist or receptor ligand in an amount effective to improve the learning or memory or both of said mammal.”

U.S. Pat. Application Publication Number 2014/0235726 discloses “a method of improving cognition in a patient with Down syndrome, which entails administering one or more β2 adrenergic receptor agonists to the patient in an amount and with a frequency effective to improve cognition of the patient as measured by contextual learning tests.”

U.S. Pat. Application Publication Number 2016/0184241 discloses “a method of improving cognition in a patient with Down syndrome, which entails intranasally administering one or more β2-ADR agonists or pharmaceutically-acceptable salts of either or both to the patient in an amount and with a frequency effective to improve cognition of the patient as measured contextual learning tests.”

### SUMMARY OF THE INVENTION

It has now been found that novel forms of the present disclosure, and compositions thereof, are useful as beta adrenergic agonists and exhibit desirable characteristics for the same. In general, salt forms or freebase forms, and pharmaceutically acceptable compositions thereof, are useful for treating or lessening the severity of a variety of diseases or disorders as described in detail herein.

### BRIEF DESCRIPTION OF THE DRAWINGS

FIG. 1A.1 depicts an XRPD pattern of Form A of compound 1.

FIG. 1A.2 depicts a DSC thermogram and TGA trace of Form A of compound 1.

FIG. 1A.3 depicts a ¹H NMR spectrum of Form A of compound 1.

FIG. 1B.1 depicts an XRPD pattern of Form B of compound 1.

FIG. 1B.2 depicts a DSC thermogram and TGA trace of Form B of compound 1.

FIG. 1B.3 depicts a ¹H NMR spectrum of Form B of compound 1.

FIG. 2A.1 depicts an XRPD pattern of Form A of compound 2.

FIG. 2A.2 depicts a DSC thermogram and TGA trace of Form A of compound 2.

FIGS. 2A.3 and 2A.4 depicts DVS plots of Form A of compound 2.

FIG. 3A.1 depicts an XRPD pattern of Form A of compound 3.

FIG. 3A.2 depicts a DSC thermogram and TGA trace of Form A of compound 3.

FIG. 4A.1 depicts an XRPD pattern of Form A of compound 4.

FIG. 4A.2 depicts a DSC thermogram and TGA trace of Form A of compound 4.

FIG. 5A.1 depicts an XRPD pattern of Form A of compound 5.

FIG. 5A.2 depicts a DSC thermogram and TGA trace of Form A of compound 5.

FIG. 5A.3 depicts a ¹H NMR spectrum of Form A of compound 5.

FIGS. 5A.4 and 5A.5 depict DVS plots of Form A of compound 5.

FIG. 6A.1 depicts an XRPD pattern of Form A of compound 6.

FIG. 6A.2 depicts a DSC thermogram and TGA trace of Form A of compound 6.

FIG. 6A.3 depicts a ¹H NMR spectrum of Form A of compound 6.

FIG. 7A.1 depicts an XRPD pattern of Form A of compound 7.

FIG. 7A.2 depicts a DSC thermogram and TGA trace of Form A of compound 7.

FIG. 7A.3 depicts a ¹H NMR spectrum of Form A of compound 7.

FIG. 8A.1 depicts an XRPD pattern of Form A of compound 8.

FIG. 8A.2 depicts a DSC thermogram and TGA trace of Form A of compound 8, wherein the DSC thermogram shows the results of two separate batches of Form A of compound 8.

FIG. 8A.3 depicts a ¹H NMR spectrum of Form A of compound 8.

FIG. 9A.1 depicts an XRPD pattern of Form A of compound 9.

FIG. 9A.2 depicts a DSC thermogram and TGA trace of Form A of compound 9.

FIG. 9A.3 depicts a ¹H NMR spectrum of Form A of compound 9.

FIGS. 9A.4 and 9A.5depict DVS plots of Form A of compound 9.

FIG. 10A.1 depicts an XRPD pattern of Form A of compound 10.

FIG. 10A.2 depicts a DSC thermogram and TGA trace of Form A of compound 10.

FIG. 10A.3 depicts a ¹H NMR spectrum of Form A of compound 10.

FIG. 11A.1 depicts an XRPD pattern of Form A of compound 11.

FIG. 11A.2 depicts a DSC thermogram and TGA trace of Form A of compound 11.

FIG. 11A.3 depicts a ¹H NMR spectrum of Form A of compound 11.

FIG. 12A.1 depicts an XRPD pattern of Form A of compound 12.

FIG. 12A.2 depicts a DSC thermogram and TGA trace of Form A of compound 12.

FIG. 13A.1 depicts an XRPD pattern of Form A of compound 13.

FIG. 13A.2 depicts a DSC thermogram and TGA trace of Form A of compound 13.

FIG. 13A.3 depicts a ¹H NMR spectrum of Form A of compound 13.

FIG. 14A.1 depicts an XRPD pattern of Form A of compound 14.

FIG. 14A.2 depicts a DSC thermogram and TGA trace of Form A of compound 14.

FIG. 14A.3 depicts a ¹H NMR spectrum of Form A of compound 14.

### DETAILED DESCRIPTION OF THE INVENTION

### General Description of Certain Aspects of the Invention

The present disclosure is based at least in part on the identification of a compound that modulates adrenergic receptors and methods of using the same to treat diseases associated with an adrenergic receptor. Disclosed herein is compound 1:

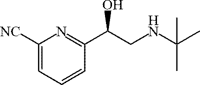

Compound 1, (S)-6-(2-(tert-butylamino)-1-hydroxyethyl)picolinonitrile, is active in a variety of assays and therapeutic models, acting as a low concentration partial agonist of the β2 adrenergic receptor. Compound 1 has been further found to demonstrate an unexpectedly high ability to cross the blood-brain barrier and accumulate in the cerebral spinal fluid.

It would be desirable to provide a solid form of compound 1 (e.g., as a freebase thereof or salt thereof) that imparts characteristics such as improved aqueous solubility, stability and ease of formulation. Accordingly, the present disclosure provides both free base forms and salt forms of compound 1.

### Free Base Forms of Compound 1

It is contemplated that compound 1 can exist in a variety of physical forms. For example, compound 1 can be in solution, suspension, or in solid form. In certain embodiments, compound 1 is in solid form. When compound 1 is in solid form, said compound may be amorphous, crystalline, or a mixture thereof. Exemplary solid forms are described in more detail below.

In some embodiments, the present disclosure provides a form of compound 1 substantially free of impurities. As used herein, the term “substantially free of impurities” means that the compound contains no significant amount of extraneous matter. Such extraneous matter may include different forms of compound 1, residual solvents, or any other impurities that may result from the preparation of, and/or isolation of, compound 1. In certain embodiments, at least about 95% by weight of a form of compound 1 is present. In still other embodiments of the disclosure, at least about 99% by weight of a form of compound 1 is present.

According to one embodiment, a form of compound 1 is present in an amount of at least about 97, 97.5, 98.0, 98.5, 99, 99.5, 99.8 weight percent where the percentages are based on the total weight of the composition. According to another embodiment, a form of compound 1 contains no more than about 3.0 area percent HPLC of total organic impurities and, in certain embodiments, no more than about 1.5 area percent HPLC total organic impurities relative to the total area of the HPLC chromatogram. In other embodiments, a form of compound 1 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 1 is also meant to include all tautomeric forms of compound 1. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 1 can exist in a variety of solid forms. Exemplary such forms include polymorphs such as those described herein.

As used herein, the term “polymorph” refers to the different crystal structures into which a compound, or a salt or solvate thereof, can crystallize.

In certain embodiments, compound 1 is a crystalline solid. In other embodiments, compound 1 is a crystalline solid substantially free of amorphous compound 1. As used herein, the term “substantially free of amorphous compound 1” means that the compound contains no significant amount of amorphous compound 1. In certain embodiments, at least about 95% by weight of crystalline compound 1 is present. In still other embodiments of the disclosure, at least about 99% by weight of crystalline compound 1 is present.

It has been found that the free base compound 1 can exist in at least two distinct polymorphic form. In certain embodiments, the present disclosure provides a polymorphic form of compound 1 referred to herein as Form A. In certain embodiments, the present disclosure provides a polymorphic form of compound 1 referred to herein as Form B.

In some embodiments, compound 1 is amorphous. In some embodiments, compound 1 is amorphous, and is substantially free of crystalline compound 1.

### Form A of Compound 1

In some embodiments, Form A of compound 1 has at least 1, 2, 3, 4 or 5 spectral peak(s) selected from the peaks listed in Table 1 below.

TABLE 1

XRPD Peak Positions for Form A of Compound 1

| Position (°2θ) | Intensity % | Position (°2θ) | Intensity % |
|---|---|---|---|
| 6.5 | 3.1 | 23.5 | 11.8 |
| 9.3 | 100.0 | 23.9 | 3.3 |
| 9.7 | 15.8 | 25.1 | 23.1 |
| 10.2 | 7.0 | 25.6 | 6.4 |
| 11.5 | 1.4 | 25.9 | 3.7 |
| 12.6 | 20.0 | 26.2 | 1.7 |
| 13.0 | 1.5 | 27.5 | 1.6 |
| 14.7 | 2.6 | 28.3 | 11.4 |
| 15.3 | 1.5 | 29.1 | 3.9 |
| 15.7 | 3.5 | 29.6 | 6.0 |
| 16.4 | 12.8 | 30.7 | 2.4 |
| 16.9 | 21.7 | 31.1 | 2.2 |
| 17.5 | 8.6 | 32.5 | 1.1 |
| 18.8 | 20.0 | 32.9 | 1.2 |
| 19.3 | 1.9 | 34.3 | 2.8 |
| 20.0 | 2.0 | 35.6 | 0.8 |
| 20.6 | 47.0 | 36.2 | 1.2 |
| 21.7 | 1.7 | 37.2 | 2.0 |
| 22.0 | 2.5 | 38.1 | 1.6 |
| 22.5 | 6.2 | 38.6 | 0.9 |

In this and all subsequent tables, the position (°2θ) is within ± 0.2.

In some embodiments, Form A of compound 1 is characterized in that it has one or more peaks in its X-ray powder diffraction pattern selected from those at about 9.3, about 12.6, about 16.9, about 18.8, about 20.6, and about 25.1 degrees 2-theta. In some embodiments, Form A of compound 1 is characterized in that it has two or more peaks in its X-ray powder diffraction pattern selected from those at about 9.3, about 12.6, about 16.9, about 18.8, about 20.6, and about 25.1 degrees 2-theta. In some embodiments, Form A of compound 1 is characterized in that it has three or more peaks in its X-ray powder diffraction pattern selected from those at about 9.3, about 12.6, about 16.9, about 18.8, about 20.6, and about 25.1 degrees 2-theta. In some embodiments, Form A of compound 1 is characterized in that it has four or more peaks in its X-ray powder diffraction pattern selected from those at about 9.3, about 12.6, about 16.9, about 18.8, about 20.6, and about 25.1 degrees 2-theta. In some embodiments, Form A of compound 1 is characterized in that it has five or more peaks in its X-ray powder diffraction pattern selected from those at about 9.3, about 12.6, about 16.9, about 18.8, about 20.6, and about 25.1 degrees 2-theta. In some embodiments, Form A of compound 1 is characterized in that it has six peaks in its X-ray powder diffraction pattern at about 9.3, about 12.6, about 16.9, about 18.8, about 20.6, and about 25.1 degrees 2-theta. As used herein, the term “about”, when used in reference to a degree 2-theta value refers to the stated value ± 0.2 degree 2-theta.

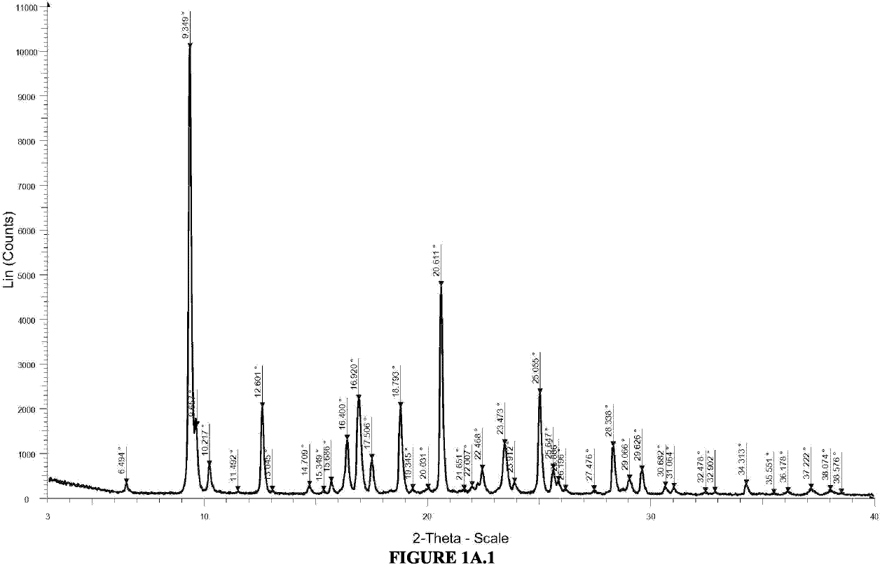

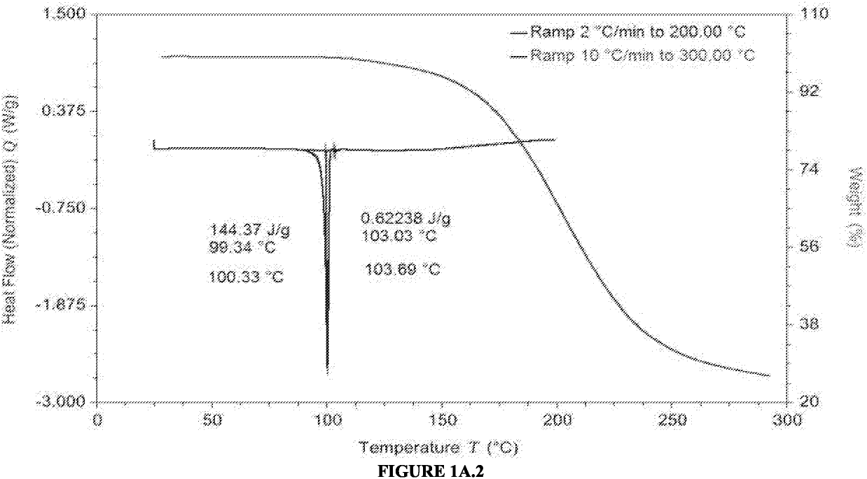

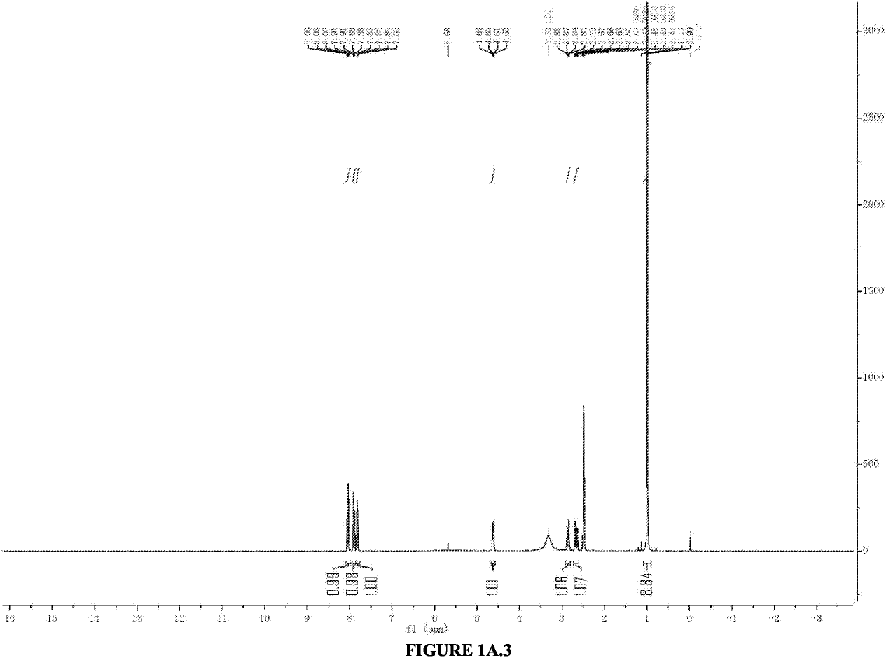

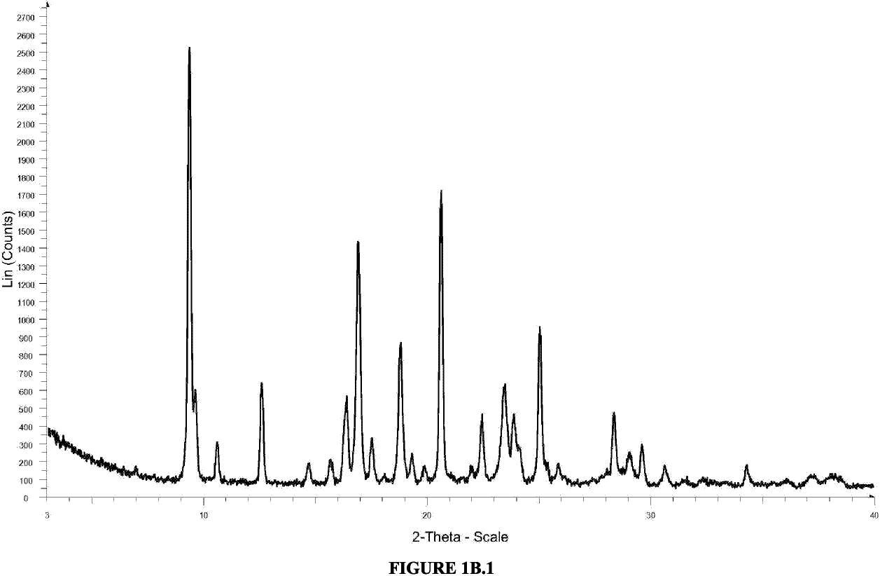

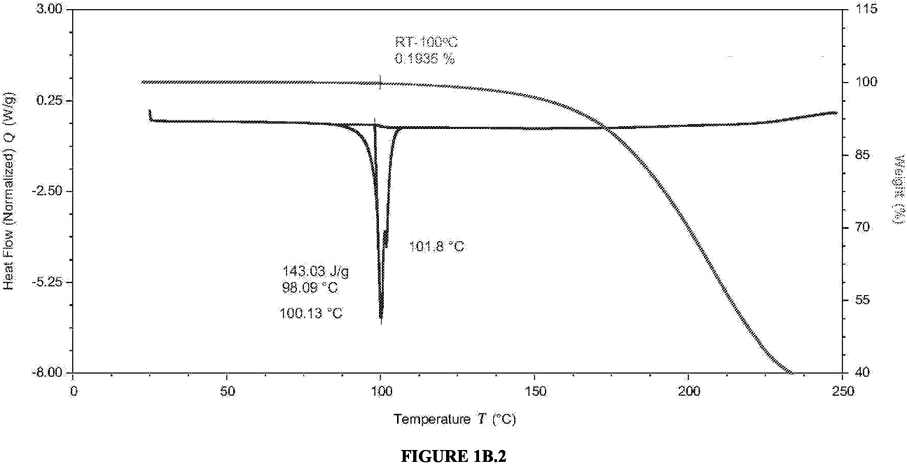

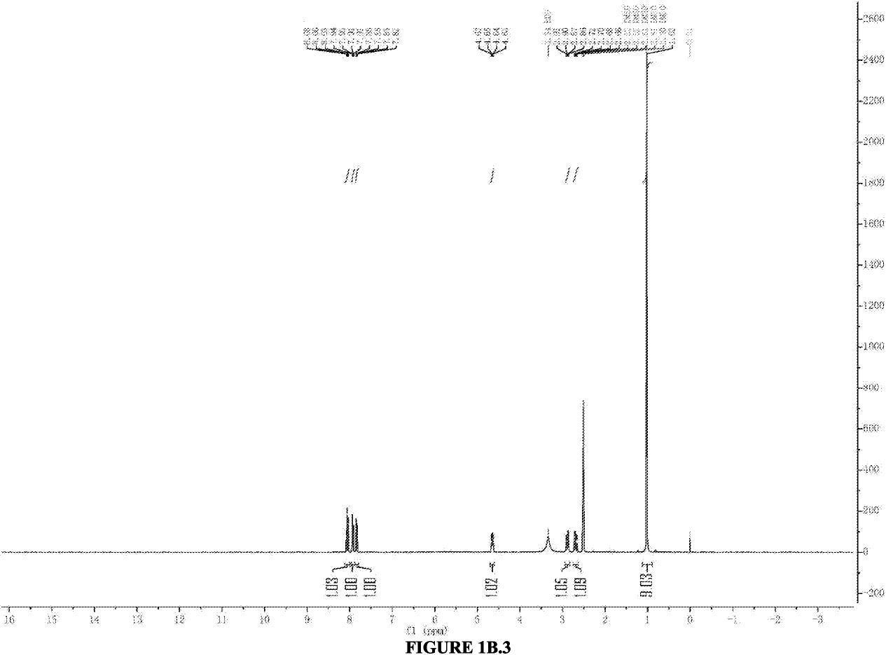

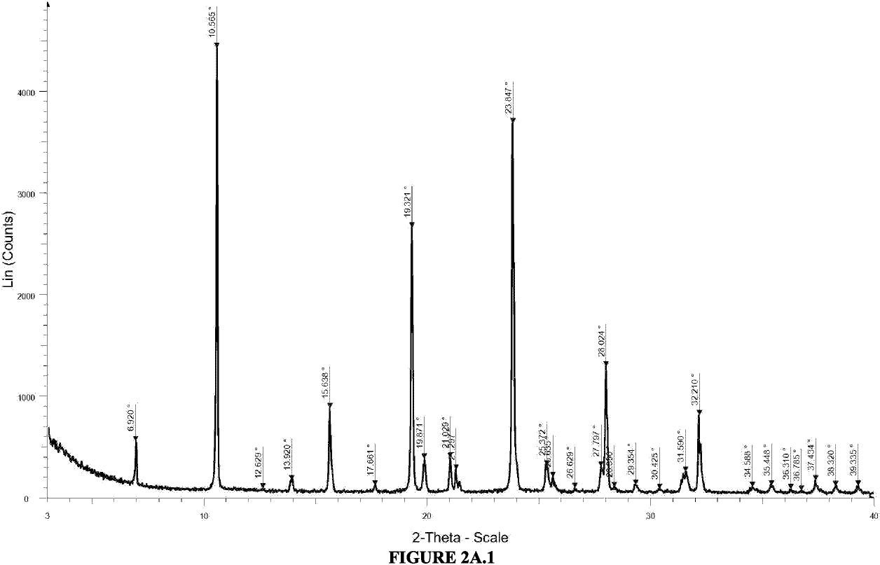

In some embodiments, Form A of compound 1 is characterized in that it has each of the spectral peaks in its X-ray powder diffraction pattern listed in Table 1 having a relative intensity greater than 10%, 20%, 30% or 40%. In certain embodiments, the X-ray powder diffraction pattern is substantially similar to the XRPD provided in FIG. 1A.1.

Methods for preparing Form A of compound 1 are described infra.

### Form B of Compound 1

In some embodiments, Form B of compound 1 has at least 1, 2, 3, 4 or 5 spectral peak(s) selected from the peaks listed in Table 2 below.

TABLE 2

XRPD Peak Positions for Form B of Compound 1

| Position (°2θ) | Intensity % | Position (°2θ) | Intensity % |
|---|---|---|---|
| 6.9 | 6.7 | 23.9 | 18.3 |
| 9.3 | 100.0 | 24.1 | 10.8 |
| 9.6 | 23.7 | 25.0 | 37.7 |
| 10.6 | 12.0 | 25.4 | 7.3 |
| 12.6 | 25.2 | 25.9 | 7.3 |
| 14.7 | 7.3 | 27.4 | 3.8 |
| 15.7 | 8.2 | 28.4 | 18.6 |
| 16.4 | 22.3 | 29.1 | 9.8 |
| 16.9 | 56.5 | 29.6 | 11.6 |
| 17.5 | 13.0 | 30.6 | 6.8 |
| 18.1 | 5.0 | 31.6 | 3.6 |
| 18.8 | 34.2 | 32.4 | 3.9 |
| 19.3 | 9.5 | 34.3 | 7.0 |
| 19.9 | 6.8 | 36.1 | 3.6 |
| 20.6 | 68.2 | 37.3 | 4.9 |
| 22.0 | 6.6 | 38.3 | 5.0 |
| 22.4 | 18.3 | 38.5 | 4.4 |
| 23.5 | 24.3 | | |

In this and all subsequent tables, the position (°2θ) is within ± 0.2.

In some embodiments, Form B of compound 1 is characterized in that it has one or more peaks in its X-ray powder diffraction pattern selected from those at about 9.3, about 12.6, about 16.9, about 18.8, about 20.6, and about 25.0 degrees 2-theta. In some embodiments, Form B of compound 1 is characterized in that it has two or more peaks in its X-ray powder diffraction pattern selected from those at about 9.3, about 12.6, about 16.9, about 18.8, about 20.6, and about 25.0 degrees 2-theta. In some embodiments, Form B of compound 1 is characterized in that it has three or more peaks in its X-ray powder diffraction pattern selected from those at about 9.3, about 12.6, about 16.9, about 18.8, about 20.6, and about 25.0 degrees 2-theta. In some embodiments, Form B of compound 1 is characterized in that it has four or more peaks in its X-ray powder diffraction pattern selected from those at about 9.3, about 12.6, about 16.9, about 18.8, about 20.6, and about 25.0 degrees 2-theta. In some embodiments, Form B of compound 1 is characterized in that it has five or more peaks in its X-ray powder diffraction pattern selected from those at about 9.3, about 12.6, about 16.9, about 18.8, about 20.6, and about 25.0 degrees 2-theta. In some embodiments, Form B of compound 1 is characterized in that it has six peaks in its X-ray powder diffraction pattern at about 9.3, about 12.6, about 16.9, about 18.8, about 20.6, and about 25.0 degrees 2-theta.

In some embodiments, Form B of compound 1 is characterized in that it has each of the spectral peaks in its X-ray powder diffraction pattern listed in Table 2 having a relative intensity greater than 10%, 20%, 30% or 40%.

In certain embodiments, the X-ray powder diffraction pattern is substantially similar to the XRPD provided in FIG. 1B.1.

Methods for preparing Form B of compound 1 are described infra.

In some embodiments, the disclosure provides compound 1:

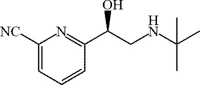

wherein said compound is crystalline. In some embodiments, the present disclosure provides compound 1, wherein said compound is substantially free of amorphous compound 1.

In some embodiments, the present disclosure provides compound 1, wherein said compound is substantially free of impurities.

In some embodiments, the present disclosure provides compound 1, wherein said compound has one or more peaks in its XRPD selected from those at about 9.3, about 12.6, about 16.9, about 18.8, about 20.6, and about 25.1 degrees 2-theta. In some such embodiments, the present disclosure provides compound 1, wherein said compound has at least two peaks in its XRPD selected from those at about 9.3, about 12.6, about 16.9, about 18.8, about 20.6, and about 25.1 degrees 2-theta. In some such embodiments, the present disclosure provides compound 1, wherein said compound is of Form A.

In some embodiments, the present disclosure provides compound 1, wherein said compound has an XRPD substantially similar to that depicted in FIG. 1A.1.

In some embodiments, the present disclosure provides compound 1, wherein said compound has one or more peaks in its XRPD selected from those at about 9.3, about 12.6, about 16.9, about 18.8, about 20.6, and about 25.0 degrees 2-theta. In some such embodiments, the present disclosure provides compound 1, wherein said compound has at least two peaks in its XRPD selected from those at about 9.3, about 12.6, about 16.9, about 18.8, about 20.6, and about 25.0 degrees 2-theta. In some such embodiments, the present disclosure provides compound 1, wherein said compound is of Form B.

In some embodiments, Form B of compound 1 is characterized in that it has each of the spectral peaks in its X-ray powder diffraction pattern listed in Table 2 having a relative intensity greater than 10%, 20%, 30% or 40%. In some embodiments, the present disclosure provides compound 1, wherein said compound has an XRPD substantially similar to that depicted in FIG. 1B.1.

In some embodiments, the present disclosure provides a composition comprising compound 1 and a pharmaceutically acceptable carrier or excipient.

In some embodiments, the present disclosure provides a method of activating an adrenergic receptor in a patient comprising administering to said patient compound 1 or a composition thereof. In some embodiments, the adrenergic receptor is selected from β1-adrenergic receptor and β2-adrenergic receptor.

In some embodiments, the present disclosure provides a method of treating a β1-adrenergic receptor or β2-adrenergic receptor mediated disease or disorder in a patient, comprising administering to said patient compound 1 or composition thereof.

### Salt Forms of Compound 1

In some embodiments, an acid and compound 1 are ionically bonded to form one of compounds 2 through 14, described below. It is contemplated that compounds 2 through 14 can exist in a variety of physical forms. For example, compounds 2 through 14 can be in solution, suspension, or in solid form. In certain embodiments, compounds 2 through 14 are in solid form. When compounds 2 through 14 are in solid form, said compounds may be amorphous, crystalline, or a mixture thereof. Exemplary such solid forms of compounds 2 through 14 are described in more detail below.

### Compound 2 (Hydrochloride Salts of Compound 1)

According to one embodiment, the present disclosure provides a hydrochloride salt of compound 1, represented by compound 2:

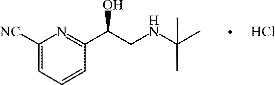

It will be appreciated by one of ordinary skill in the art that the hydrochloric acid and compound 1 are ionically bonded to form compound 2. It is contemplated that compound 2 can exist in a variety of physical forms. For example, compound 2 can be in solution, suspension, or in solid form. In certain embodiments, compound 2 is in solid form. When compound 2 is in solid form, said compound may be amorphous, crystalline, or a mixture thereof. Exemplary solid forms are described in more detail below.

In some embodiments, the present disclosure provides a form of compound 2 substantially free of impurities. As used herein, the term “substantially free of impurities” means that the compound contains no significant amount of extraneous matter. Such extraneous matter may include different forms of compound 2, residual solvents, or any other impurities that may result from the preparation of, and/or isolation of, compound 2. In certain embodiments, at least about 95% by weight of a form of compound 2 is present. In still other embodiments of the disclosure, at least about 99% by weight of a form of compound 2 is present.

According to one embodiment, a form of compound 2 is present in an amount of at least about 97, 97.5, 98.0, 98.5, 99, 99.5, 99.8 weight percent where the percentages are based on the total weight of the composition. According to another embodiment, a form of compound 2 contains no more than about 3.0 area percent HPLC of total organic impurities and, in certain embodiments, no more than about 1.5 area percent HPLC total organic impurities relative to the total area of the HPLC chromatogram. In other embodiments, a form of compound 2 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 2 is also meant to include all tautomeric forms of compound 2. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 2 can exist in a variety of solid forms. Exemplary such forms include polymorphs such as those described herein.

In certain embodiments, compound 2 is a crystalline solid. In other embodiments, compound 2 is a crystalline solid substantially free of amorphous compound 2. As used herein, the term “substantially free of amorphous compound 2” means that the compound contains no significant amount of amorphous compound 2. In certain embodiments, at least about 95% by weight of crystalline compound 2 is present. In still other embodiments of the disclosure, at least about 99% by weight of crystalline compound 2 is present.

It has been found that compound 2 can exist in at least one distinct polymorphic form. In certain embodiments, the present disclosure provides a polymorphic form of compound 2 referred to herein as Form A. In certain embodiments, the present disclosure provides a polymorphic form of compound 2 referred to herein as Form B. In certain embodiments, the present disclosure provides a polymorphic form of compound 2 referred to herein as Form C.

In some embodiments, compound 2 is amorphous. In some embodiments, compound 2 is amorphous, and is substantially free of crystalline compound 2.

### Form A of Compound 2

In some embodiments, Form A of compound 2 has at least 1, 2, 3, 4 or 5 spectral peak(s) selected from the peaks listed in Table 3 below.

TABLE 3

XRPD Peak Positions for Form A of Compound 2

| Position (°2θ) | Intensity % | Position (°2θ) | Intensity % |
|---|---|---|---|
| 6.9 | 12.4 | 27.8 | 6.7 |
| 10.6 | 100.0 | 28.0 | 29.0 |
| 12.6 | 1.8 | 28.4 | 2.1 |
| 13.9 | 3.6 | 29.4 | 2.6 |
| 15.6 | 19.7 | 30.4 | 1.7 |
| 17.7 | 2.5 | 31.6 | 5.5 |
| 19.3 | 59.9 | 32.2 | 18.1 |
| 19.9 | 8.5 | 34.6 | 2.2 |
| 21.0 | 8.6 | 35.4 | 2.5 |
| 21.3 | 6.0 | 36.3 | 1.6 |
| 23.8 | 83.2 | 36.8 | 1.3 |
| 25.4 | 6.9 | 37.4 | 3.6 |
| 25.6 | 4.4 | 38.3 | 2.3 |
| 26.6 | 2.0 | 39.3 | 2.5 |

In this and all subsequent tables, the position (°2θ) is within ± 0.2.

In some embodiments, Form A of compound 2 is characterized in that it has one or more peaks in its X-ray powder diffraction pattern selected from those at about 10.6, about 15.6, about 19.3, about 23.8, about 28.0, and about 32.2 degrees 2-theta. In some embodiments, Form A of compound 2 is characterized in that it has two or more peaks in its X-ray powder diffraction pattern selected from those at about 10.6, about 15.6, about 19.3, about 23.8, about 28.0, and about 32.2 degrees 2-theta. In some embodiments, Form A of compound 2 is characterized in that it has three or more peaks in its X-ray powder diffraction pattern selected from those at about 10.6, about 15.6, about 19.3, about 23.8, about 28.0, and about 32.2 degrees 2-theta. In some embodiments, Form A of compound 2 is characterized in that it has four or more peaks in its X-ray powder diffraction pattern selected from those at about 10.6, about 15.6, about 19.3, about 23.8, about 28.0, and about 32.2 degrees 2-theta. In some embodiments, Form A of compound 2 is characterized in that it has five or more peaks in its X-ray powder diffraction pattern selected from those at about 10.6, about 15.6, about 19.3, about 23.8, about 28.0, and about 32.2 degrees 2-theta. In some embodiments, Form A of compound 2 is characterized in that it has six peaks in its X-ray powder diffraction pattern selected from those at about 10.6, about 15.6, about 19.3, about 23.8, about 28.0, and about 32.2 degrees 2-theta.

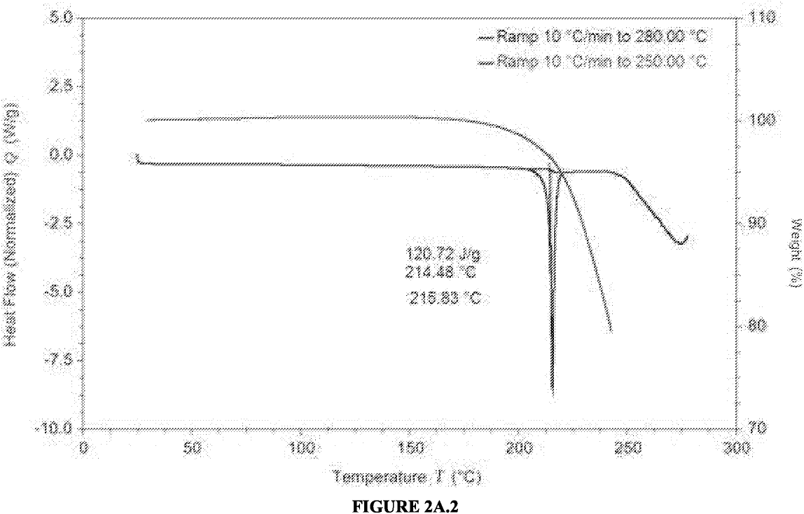

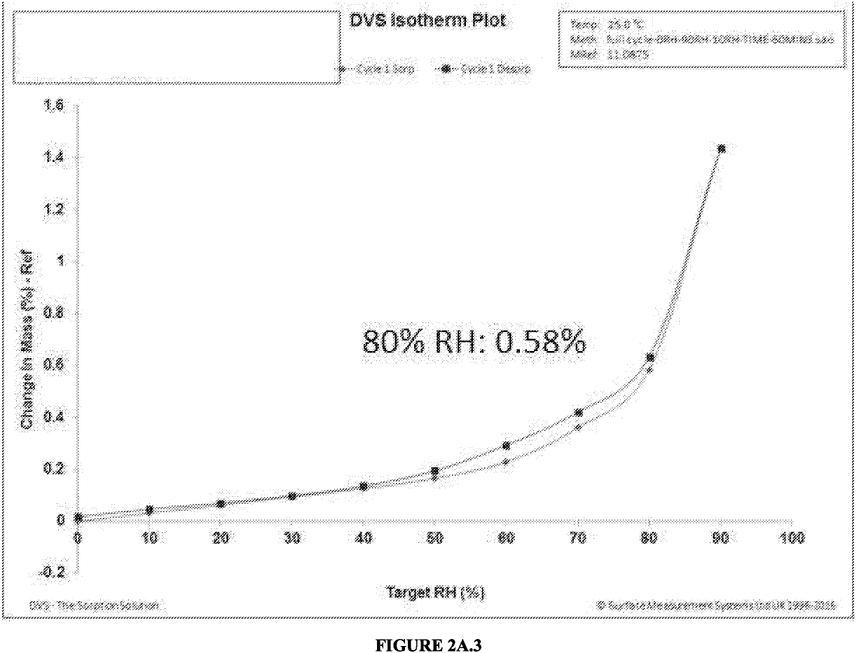

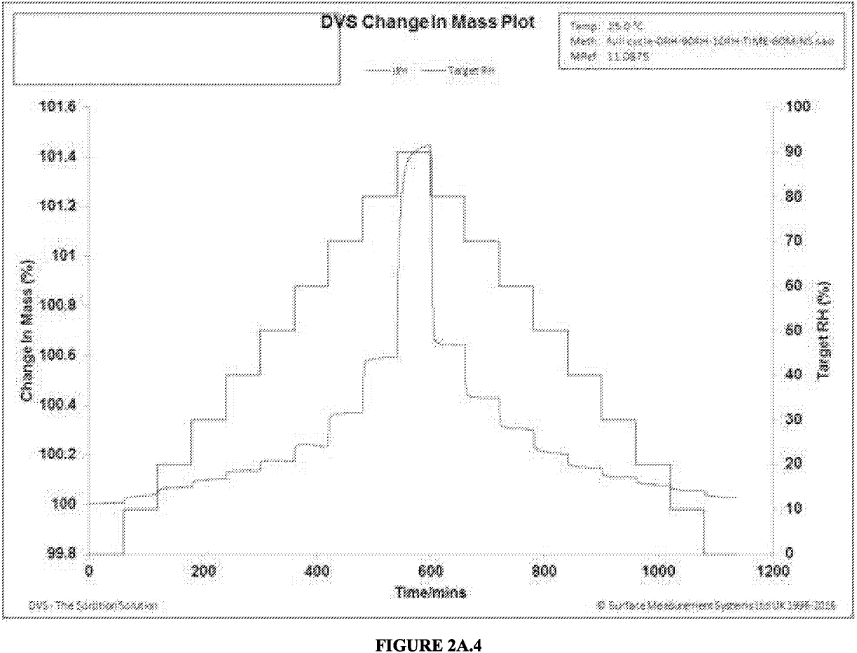

In some embodiments, Form A of compound 2 is characterized in that it has each of the spectral peaks in its X-ray powder diffraction pattern listed in Table 3 having a relative intensity greater than 10%, 20%, 30% or 40%. In certain embodiments, the X-ray powder diffraction pattern is substantially similar to the XRPD provided in FIG. 2A.1.

Methods for preparing Form A of compound 2 are described infra.

In some embodiments, the disclosure provides compound 2:

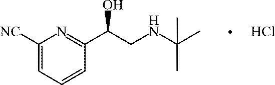

wherein said compound is crystalline. In some embodiments, the present disclosure provides compound 2, wherein said compound is substantially free of amorphous compound 2.

In some embodiments, the present disclosure provides compound 2, wherein said compound is substantially free of impurities.

In some embodiments, the present disclosure provides compound 2, wherein said compound has one or more peaks in its XRPD selected from those at about 10.6, about 15.6, about 19.3, about 23.8, about 28.0, and about 32.2 degrees 2-theta. In some such embodiments, the present disclosure provides compound 2, wherein said compound has at least two peaks in its XRPD selected from those at about 10.6, about 15.6, about 19.3, about 23.8, about 28.0, and about 32.2 degrees 2-theta. In some such embodiments, the present disclosure provides compound 2, wherein said compound is of Form A.

In some embodiments, the present disclosure provides compound 2, wherein said compound has an XRPD substantially similar to that depicted in FIG. 2A.1.

In some embodiments, the present disclosure provides a composition comprising compound 2 and a pharmaceutically acceptable carrier or excipient.

In some embodiments, the present disclosure provides a method of activating an adrenergic receptor in a patient comprising administering to said patient compound 2 or a composition thereof. In some embodiments, the adrenergic receptor is selected from β1-adrenergic receptor and β2-adrenergic receptor.

In some embodiments, the present disclosure provides a method of treating a β1-adrenergic receptor or β2-adrenergic receptor mediated disease or disorder in a patient, comprising administering to said patient compound 2 or composition thereof.

### Compound 3 (Sulfate Salts of Compound 1)

According to one embodiment, the present disclosure provides a sulfate salt of compound 1, represented by compound 3:

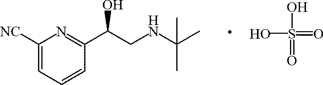

It will be appreciated by one of ordinary skill in the art that the sulfuric acid and compound 1 are ionically bonded to form compound 3. It is contemplated that compound 3 can exist in a variety of physical forms. For example, compound 3 can be in solution, suspension, or in solid form. In certain embodiments, compound 3 is in solid form. When compound 3 is in solid form, said compound may be amorphous, crystalline, or a mixture thereof. Exemplary solid forms are described in more detail below.

In some embodiments, the present disclosure provides a form of compound 3 substantially free of impurities. As used herein, the term “substantially free of impurities” means that the compound contains no significant amount of extraneous matter. Such extraneous matter may include different forms of compound 3, residual solvents, or any other impurities that may result from the preparation of, and/or isolation of, compound 3. In certain embodiments, at least about 95% by weight of a form of compound 3 is present. In still other embodiments of the disclosure, at least about 99% by weight of a form of compound 3 is present.

According to one embodiment, a form of compound 3 is present in an amount of at least about 97, 97.5, 98.0, 98.5, 99, 99.5, 99.8 weight percent where the percentages are based on the total weight of the composition. According to another embodiment, a form of compound 3 contains no more than about 3.0 area percent HPLC of total organic impurities and, in certain embodiments, no more than about 1.5 area percent HPLC total organic impurities relative to the total area of the HPLC chromatogram. In other embodiments, a form of compound 3 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 3 is also meant to include all tautomeric forms of compound 3. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 3 can exist in a variety of solid forms. Exemplary such forms include polymorphs such as those described herein.

In certain embodiments, compound 3 is a crystalline solid. In other embodiments, compound 3 is a crystalline solid substantially free of amorphous compound 3. As used herein, the term “substantially free of amorphous compound 3” means that the compound contains no significant amount of amorphous compound 3. In certain embodiments, at least about 95% by weight of crystalline compound 3 is present. In still other embodiments of the disclosure, at least about 99% by weight of crystalline compound 3 is present.

It has been found that compound 3 can exist in at least one distinct polymorphic form. In certain embodiments, the present disclosure provides a polymorphic form of compound 3 referred to herein as Form A.

In some embodiments, compound 3 is amorphous. In some embodiments, compound 3 is amorphous, and is substantially free of crystalline compound 3.

### Form A of Compound 3

In some embodiments, Form A of compound 3 has at least 1, 2, 3, 4 or 5 spectral peak(s) selected from the peaks listed in Table 4 below.

TABLE 4

XRPD Peak Positions for Form A of Compound 3

| Position (°2θ) | Intensity % | Position (°2θ) | Intensity % |
|---|---|---|---|
| 6.4 | 60.8 | 25.8 | 5.0 |
| 10.2 | 6.0 | 26.4 | 5.1 |
| 11.1 | 100.0 | 26.9 | 8.2 |
| 12.2 | 5.8 | 27.9 | 6.6 |
| 13.3 | 12.7 | 28.9 | 5.7 |
| 15.8 | 15.9 | 30.5 | 4.9 |
| 16.1 | 37.7 | 31.0 | 5.3 |
| 17.8 | 18.7 | 31.4 | 4.7 |
| 18.4 | 35.7 | 31.7 | 5.8 |
| 19.3 | 5.4 | 31.9 | 7.4 |
| 21.3 | 12.4 | 32.4 | 6.0 |
| 21.9 | 32.7 | 33.6 | 7.4 |
| 22.7 | 39.1 | 34.9 | 4.6 |
| 23.4 | 15.5 | 35.4 | 4.1 |
| 23.8 | 73.9 | 36.8 | 5.3 |
| 24.7 | 13.3 | 37.4 | 3.8 |

In this and all subsequent tables, the position (°2θ) is within ± 0.2.

In some embodiments, Form A of compound 3 is characterized in that it has one or more peaks in its X-ray powder diffraction pattern selected from those at about 6.4, about 11.1, about 16.1, about 18.4, about 21.9, about 22.7, and about 23.8 degrees 2-theta. In some embodiments, Form A of compound 3 is characterized in that it has two or more peaks in its X-ray powder diffraction pattern selected from those at about 6.4, about 11.1, about 16.1, about 18.4, about 21.9, about 22.7, and about 23.8 degrees 2-theta. In some embodiments, Form A of compound 3 is characterized in that it has three or more peaks in its X-ray powder diffraction pattern selected from those at about 6.4, about 11.1, about 16.1, about 18.4, about 21.9, about 22.7, and about 23.8 degrees 2-theta. In some embodiments, Form A of compound 3 is characterized in that it has four or more peaks in its X-ray powder diffraction pattern selected from those at about 6.4, about 11.1, about 16.1, about 18.4, about 21.9, about 22.7, and about 23.8 degrees 2-theta. In some embodiments, Form A of compound 3 is characterized in that it has five or more peaks in its X-ray powder diffraction pattern selected from those at about 6.4, about 11.1, about 16.1, about 18.4, about 21.9, about 22.7, and about 23.8 degrees 2-theta. In some embodiments, Form A of compound 3 is characterized in that it has six or more peaks in its X-ray powder diffraction pattern selected from those at about 6.4, about 11.1, about 16.1, about 18.4, about 21.9, about 22.7, and about 23.8 degrees 2-theta. In some embodiments, Form A of compound 3 is characterized in that it has seven peaks in its X-ray powder diffraction pattern selected from those at about 6.4, about 11.1, about 16.1, about 18.4, about 21.9, about 22.7, and about 23.8 degrees 2-theta.

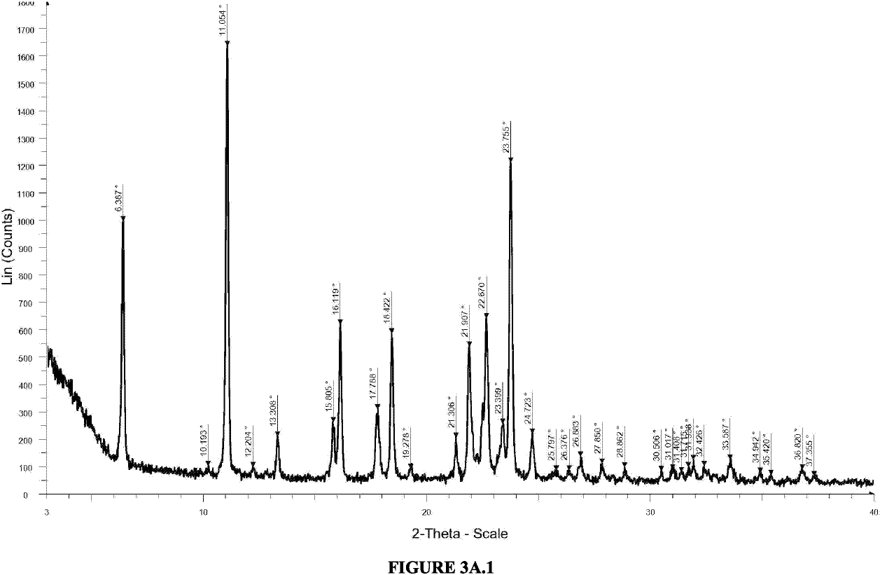

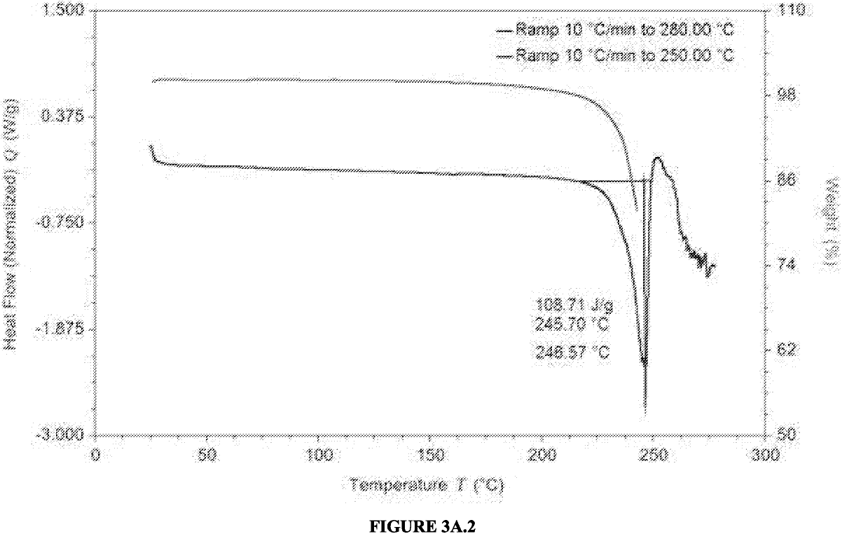

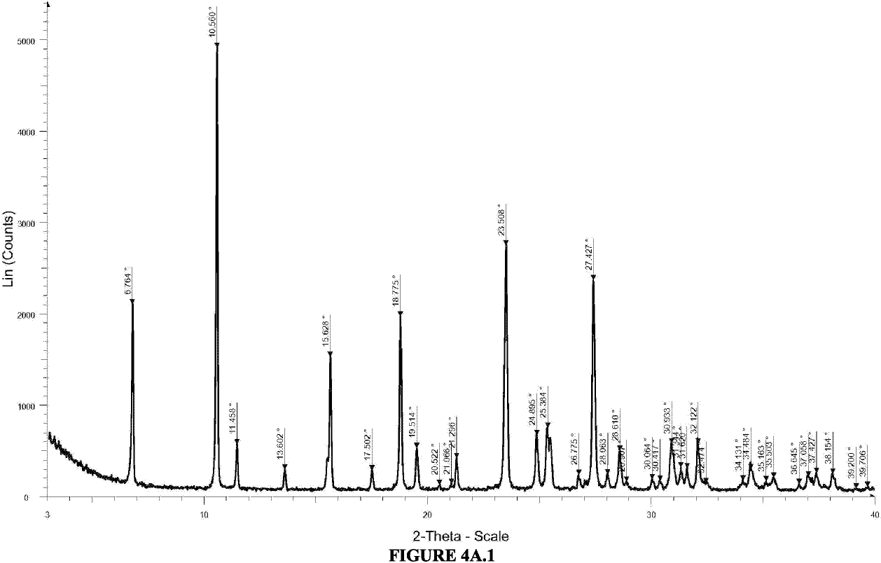

In some embodiments, Form A of compound 3 is characterized in that it has each of the spectral peaks in its X-ray powder diffraction pattern listed in Table 4 having a relative intensity greater than 10%, 20%, 30% or 40%. In certain embodiments, the X-ray powder diffraction pattern is substantially similar to the XRPD provided in FIG. 3A.1.

Methods for preparing Form A of compound 3 are described inƒra.

In some embodiments, the disclosure provides compound 3:

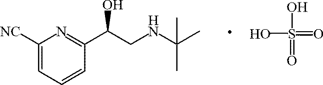

wherein said compound is crystalline. In some embodiments, the present disclosure provides compound 3, wherein said compound is substantially free of amorphous compound 3.

In some embodiments, the present disclosure provides compound 3, wherein said compound is substantially free of impurities.

In some embodiments, the present disclosure provides compound 3, wherein said compound has one or more peaks in its XRPD selected from those at about 6.4, about 11.1, about 16.1, about 18.4, about 21.9, about 22.7, and about 23.8 degrees 2-theta. In some such embodiments, the present disclosure provides compound 3, wherein said compound has at least two peaks in its XRPD selected from those at about 6.4, about 11.1, about 16.1, about 18.4, about 21.9, about 22.7, and about 23.8 degrees 2-theta. In some such embodiments, the present disclosure provides compound 3, wherein said compound is of Form A.

In some embodiments, the present disclosure provides compound 3, wherein said compound has an XRPD substantially similar to that depicted in FIG. 3A.1.

In some embodiments, the present disclosure provides a composition comprising compound 3 and a pharmaceutically acceptable carrier or excipient.

In some embodiments, the present disclosure provides a method of activating an adrenergic receptor in a patient comprising administering to said patient compound 3 or a composition thereof. In some embodiments, the adrenergic receptor is selected from β1-adrenergic receptor and β2-adrenergic receptor.

In some embodiments, the present disclosure provides a method of treating a β1-adrenergic receptor or β2-adrenergic receptor mediated disease or disorder in a patient, comprising administering to said patient compound 3 or composition thereof.

### Compound 4 (Hydrobromide Salts of Compound 1)

According to one embodiment, the present disclosure provides a hydrobromide salt of compound 1, represented by compound 4:

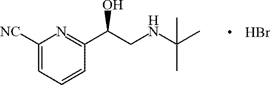

It will be appreciated by one of ordinary skill in the art that the hydrobromic acid and compound 1 are ionically bonded to form compound 4. It is contemplated that compound 4 can exist in a variety of physical forms. For example, compound 4 can be in solution, suspension, or in solid form. In certain embodiments, compound 4 is in solid form. When compound 4 is in solid form, said compound may be amorphous, crystalline, or a mixture thereof. Exemplary solid forms are described in more detail below.

In some embodiments, the present disclosure provides a form of compound 4 substantially free of impurities. As used herein, the term “substantially free of impurities” means that the compound contains no significant amount of extraneous matter. Such extraneous matter may include different forms of compound 4, residual solvents, or any other impurities that may result from the preparation of, and/or isolation of, compound 4. In certain embodiments, at least about 95% by weight of a form of compound 4 is present. In still other embodiments of the disclosure, at least about 99% by weight of a form of compound 4 is present.

According to one embodiment, a form of compound 4 is present in an amount of at least about 97, 97.5, 98.0, 98.5, 99, 99.5, 99.8 weight percent where the percentages are based on the total weight of the composition. According to another embodiment, a form of compound 4 contains no more than about 3.0 area percent HPLC of total organic impurities and, in certain embodiments, no more than about 1.5 area percent HPLC total organic impurities relative to the total area of the HPLC chromatogram. In other embodiments, a form of compound 4 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 4 is also meant to include all tautomeric forms of compound 4. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 4 can exist in a variety of solid forms. Exemplary such forms include polymorphs such as those described herein.

In certain embodiments, compound 4 is a crystalline solid. In other embodiments, compound 4 is a crystalline solid substantially free of amorphous compound 4. As used herein, the term “substantially free of amorphous compound 4” means that the compound contains no significant amount of amorphous compound 4. In certain embodiments, at least about 95% by weight of crystalline compound 4 is present. In still other embodiments of the disclosure, at least about 99% by weight of crystalline compound 4 is present.

It has been found that compound 4 can exist in at least one distinct polymorphic form. In certain embodiments, the present disclosure provides a polymorphic form of compound 4 referred to herein as Form A.

In some embodiments, compound 4 is amorphous. In some embodiments, compound 4 is amorphous, and is substantially free of crystalline compound 4.

### Form A of Compound 4

In some embodiments, Form A of compound 4 has at least 1, 2, 3, 4 or 5 spectral peak(s) selected from the peaks listed in Table 5 below.

TABLE 5

XRPD Peak Positions for Form A of Compound 4

| Position (°2θ) | Intensity % | Position (°2θ) | Intensity % |
|---|---|---|---|
| 6.8 | 42.7 | 28.9 | 3.2 |
| 10.6 | 100.0 | 30.1 | 3.7 |
| 11.5 | 11.5 | 30.4 | 3.4 |
| 13.6 | 5.9 | 30.9 | 11.7 |
| 15.6 | 31.1 | 31.3 | 6.3 |
| 17.5 | 5.6 | 31.6 | 6.1 |
| 18.8 | 40.0 | 32.1 | 11.7 |
| 19.5 | 10.7 | 32.5 | 2.8 |
| 20.5 | 2.4 | 34.1 | 3.4 |
| 21.1 | 2.6 | 34.5 | 6.0 |
| 21.3 | 8.4 | 35.2 | 3.3 |
| 23.5 | 55.9 | 35.5 | 4.0 |
| 24.9 | 13.5 | 36.7 | 2.6 |
| 25.4 | 15.2 | 37.1 | 4.3 |
| 26.8 | 4.7 | 37.4 | 5.1 |
| 27.4 | 48.2 | 38.2 | 5.0 |
| 28.1 | 4.8 | 39.2 | 1.6 |
| 28.6 | 10.0 | 39.7 | 2.1 |

In this and all subsequent tables, the position (°2θ) is within ± 0.2.

In some embodiments, Form A of compound 4 is characterized in that it has one or more peaks in its X-ray powder diffraction pattern selected from those at about 6.8, about 10.6, about 15.6, about 18.8, about 23.5, and about 27.4 degrees 2-theta. In some embodiments, Form A of compound 4 is characterized in that it has two or more peaks in its X-ray powder diffraction pattern selected from those at about 6.8, about 10.6, about 15.6, about 18.8, about 23.5, and about 27.4 degrees 2-theta. In some embodiments, Form A of compound 4 is characterized in that it has three or more peaks in its X-ray powder diffraction pattern selected from those at about 6.8, about 10.6, about 15.6, about 18.8, about 23.5, and about 27.4 degrees 2-theta. In some embodiments, Form A of compound 4 is characterized in that it has four or more peaks in its X-ray powder diffraction pattern selected from those at about 6.8, about 10.6, about 15.6, about 18.8, about 23.5, and about 27.4 degrees 2-theta. In some embodiments, Form A of compound 4 is characterized in that it has five or more peaks in its X-ray powder diffraction pattern selected from those at about 6.8, about 10.6, about 15.6, about 18.8, about 23.5, and about 27.4 degrees 2-theta. In some embodiments, Form A of compound 4 is characterized in that it has six peaks in its X-ray powder diffraction pattern selected from those at about 6.8, about 10.6, about 15.6, about 18.8, about 23.5, and about 27.4 degrees 2-theta.

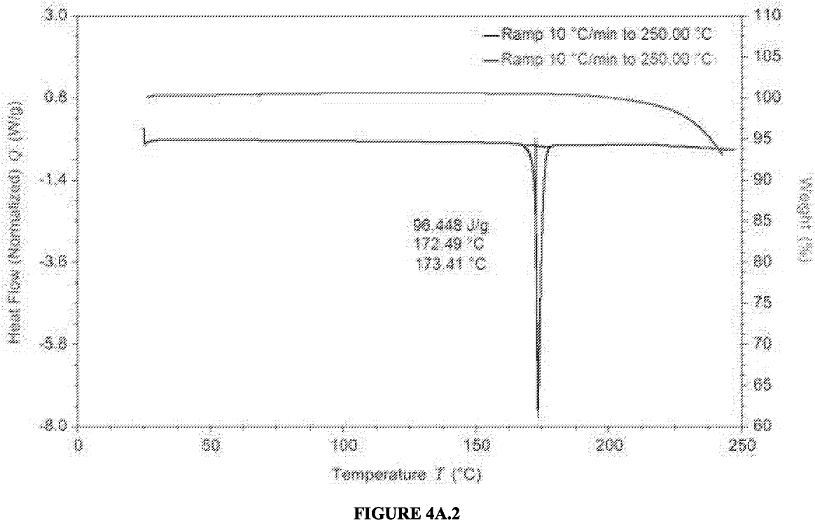

In some embodiments, Form A of compound 4 is characterized in that it has each of the spectral peaks in its X-ray powder diffraction pattern listed in Table 5 having a relative intensity greater than 10%, 20%, 30% or 40%. In certain embodiments, the X-ray powder diffraction pattern is substantially similar to the XRPD provided in FIG. 4A.1.

Methods for preparing Form A of compound 4 are described infra.

In some embodiments, the disclosure provides compound 4:

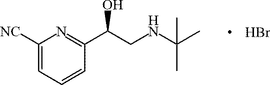

wherein said compound is crystalline. In some embodiments, the present disclosure provides compound 4, wherein said compound is substantially free of amorphous compound 4.

In some embodiments, the present disclosure provides compound 4, wherein said compound is substantially free of impurities.

In some embodiments, the present disclosure provides compound 4, wherein said compound has one or more peaks in its XRPD selected from those at about 6.8, about 10.6, about 15.6, about 18.8, about 23.5, and about 27.4 degrees 2-theta. In some such embodiments, the present disclosure provides compound 4, wherein said compound has at least two peaks in its XRPD selected from those at about 6.8, about 10.6, about 15.6, about 18.8, about 23.5, and about 27.4 degrees 2-theta. In some such embodiments, the present disclosure provides compound 4, wherein said compound is of Form A.

In some embodiments, the present disclosure provides compound 4, wherein said compound has an XRPD substantially similar to that depicted in FIG. 4A.1.

In some embodiments, the present disclosure provides a composition comprising compound 4 and a pharmaceutically acceptable carrier or excipient.

In some embodiments, the present disclosure provides a method of activating an adrenergic receptor in a patient comprising administering to said patient compound 4 or a composition thereof. In some embodiments, the adrenergic receptor is selected from β1-adrenergic receptor and β2-adrenergic receptor.

In some embodiments, the present disclosure provides a method of treating a β1-adrenergic receptor or β2-adrenergic receptor mediated disease or disorder in a patient, comprising administering to said patient compound 4 or composition thereof.

### Compound 5 (Tosylate Salts of Compound 1)

According to one embodiment, the present disclosure provides a tosylate salt of compound 1, represented by compound 5:

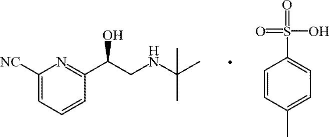

It will be appreciated by one of ordinary skill in the art that the para-toluenesulfonic acid and compound 1 are ionically bonded to form compound 5. It is contemplated that compound 5 can exist in a variety of physical forms. For example, compound 5 can be in solution, suspension, or in solid form. In certain embodiments, compound 5 is in solid form. When compound 5 is in solid form, said compound may be amorphous, crystalline, or a mixture thereof. Exemplary solid forms are described in more detail below.

In some embodiments, the present disclosure provides a form of compound 5 substantially free of impurities. As used herein, the term “substantially free of impurities” means that the compound contains no significant amount of extraneous matter. Such extraneous matter may include different forms of compound 5, residual solvents, or any other impurities that may result from the preparation of, and/or isolation of, compound 5. In certain embodiments, at least about 95% by weight of a form of compound 5 is present. In still other embodiments of the disclosure, at least about 99% by weight of a form of compound 5 is present.

According to one embodiment, a form of compound 5 is present in an amount of at least about 97, 97.5, 98.0, 98.5, 99, 99.5, 99.8 weight percent where the percentages are based on the total weight of the composition. According to another embodiment, a form of compound 5 contains no more than about 3.0 area percent HPLC of total organic impurities and, in certain embodiments, no more than about 1.5 area percent HPLC total organic impurities relative to the total area of the HPLC chromatogram. In other embodiments, a form of compound 5 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 5 is also meant to include all tautomeric forms of compound 5. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 5 can exist in a variety of solid forms. Exemplary such forms include polymorphs such as those described herein.

In certain embodiments, compound 5 is a crystalline solid. In other embodiments, compound 5 is a crystalline solid substantially free of amorphous compound 5. As used herein, the term “substantially free of amorphous compound 5” means that the compound contains no significant amount of amorphous compound 5. In certain embodiments, at least about 95% by weight of crystalline compound 5 is present. In still other embodiments of the disclosure, at least about 99% by weight of crystalline compound 5 is present.

It has been found that compound 5 can exist in at least one distinct polymorphic form. In certain embodiments, the present disclosure provides a polymorphic form of compound 5 referred to herein as Form A.

In some embodiments, compound 5 is amorphous. In some embodiments, compound 5 is amorphous, and is substantially free of crystalline compound 5.

### Form A of Compound 5

In some embodiments, Form A of compound 5 has at least 1, 2, 3, 4 or 5 spectral peak(s) selected from the peaks listed in Table 6 below.

TABLE 6

XRPD Peak Positions for Form A of Compound 5

| Position (°2θ) | Intensity % | Position (°2θ) | Intensity % |
|---|---|---|---|
| 3.2 | 8.2 | 23.8 | 5.6 |
| 7.1 | 100.0 | 24.3 | 2.7 |
| 7.6 | 25.5 | 24.9 | 5.2 |
| 9.9 | 10.0 | 25.1 | 12.9 |
| 13.6 | 4.6 | 25.7 | 2.9 |
| 14.1 | 11.8 | 26.6 | 6.0 |
| 15.4 | 24.5 | 27.6 | 7.4 |
| 15.9 | 9.8 | 27.8 | 6.0 |
| 17.0 | 15.6 | 28.6 | 1.7 |
| 17.4 | 3.1 | 28.8 | 2.2 |
| 18.8 | 4.0 | 29.8 | 5.4 |
| 19.5 | 6.5 | 31.2 | 2.6 |
| 19.9 | 28.0 | 33.1 | 2.5 |
| 20.8 | 9.4 | 34.4 | 1.9 |
| 21.1 | 11.1 | 35.1 | 2.8 |
| 21.8 | 7.2 | 36.3 | 4.0 |
| 22.3 | 3.5 | 37.1 | 1.2 |
| 22.7 | 4.7 | 38.1 | 1.8 |
| 23.3 | 31.9 | 38.5 | 2.3 |

In this and all subsequent tables, the position (°2θ) is within ± 0.2.

In some embodiments, Form A of compound 5 is characterized in that it has one or more peaks in its X-ray powder diffraction pattern selected from those at about 7.1, about 7.6, about 15.4, about 19.9, and about 23.3 degrees 2-theta. In some embodiments, Form A of compound 5 is characterized in that it has two or more peaks in its X-ray powder diffraction pattern selected from those at about 7.094, about 7.644, about 15.432, about 19.92, and about 23254. In some embodiments, Form A of compound 5 is characterized in that it has three or more peaks in its X-ray powder diffraction pattern selected from those at about 7.1, about 7.6, about 15.4, about 19.9, and about 23.3 degrees 2-theta. In some embodiments, Form A of compound 5 is characterized in that it has four or more peaks in its X-ray powder diffraction pattern selected from those at about 7.1, about 7.6, about 15.4, about 19.9, and about 23.3 degrees 2-theta. In some embodiments, Form A of compound 5 is characterized in that it has five peaks in its X-ray powder diffraction pattern selected from those at about 7.1, about 7.6, about 15.4, about 19.9, and about 23.3 degrees 2-theta.

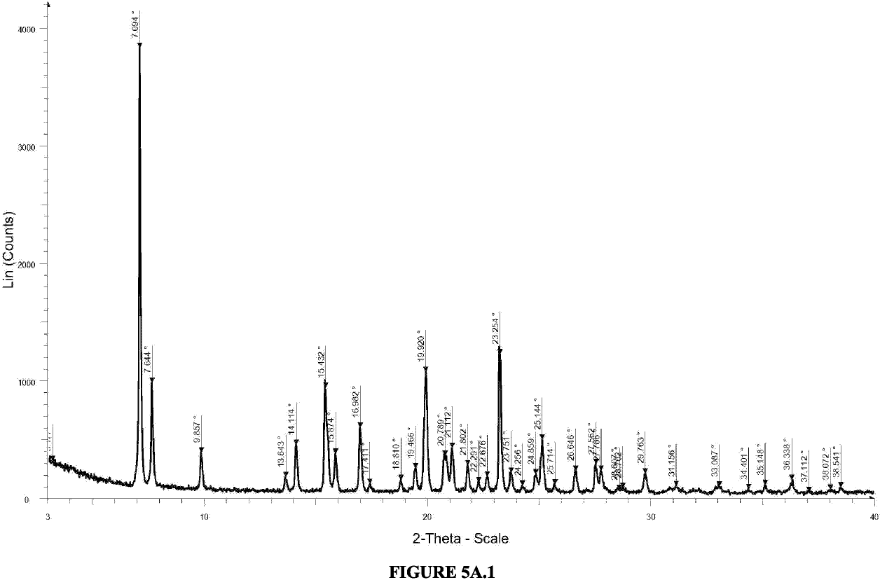

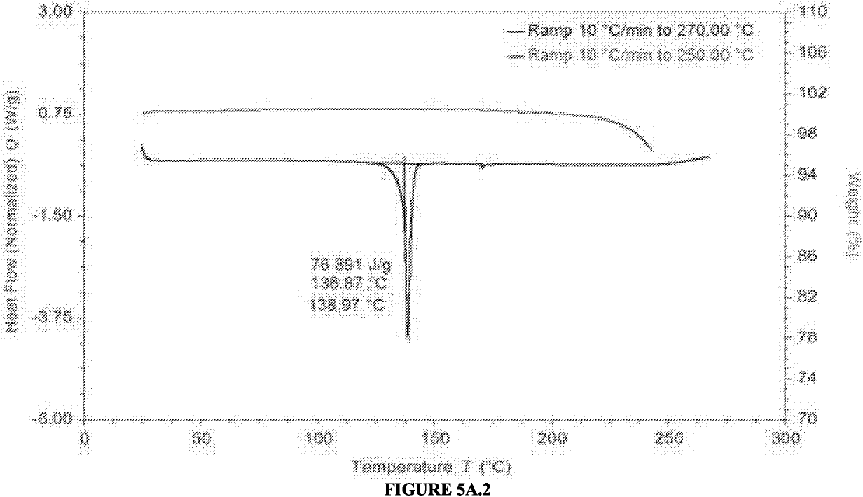

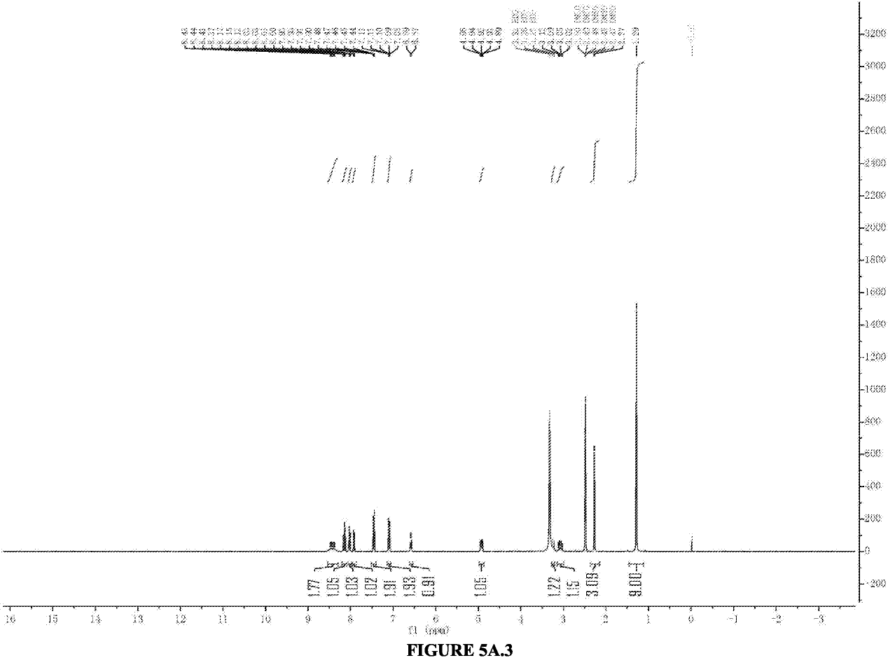

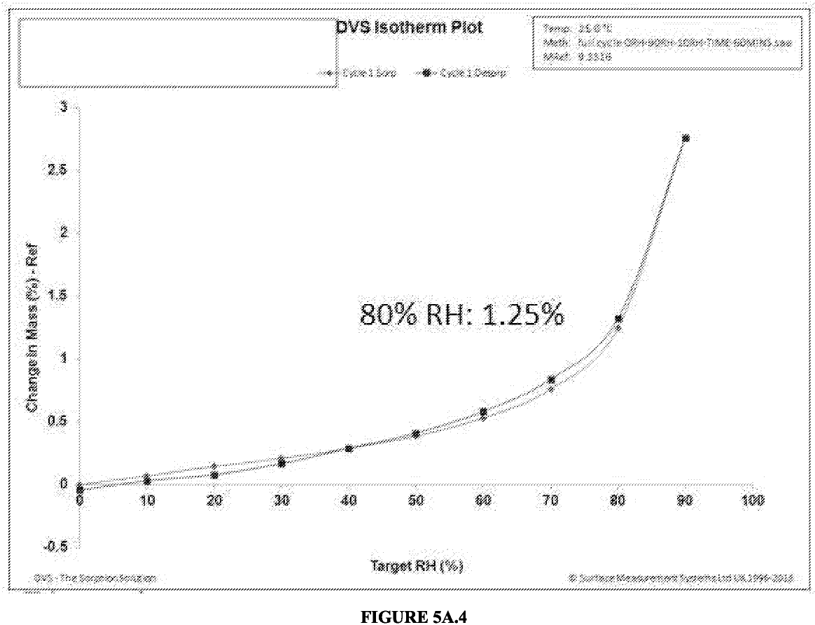

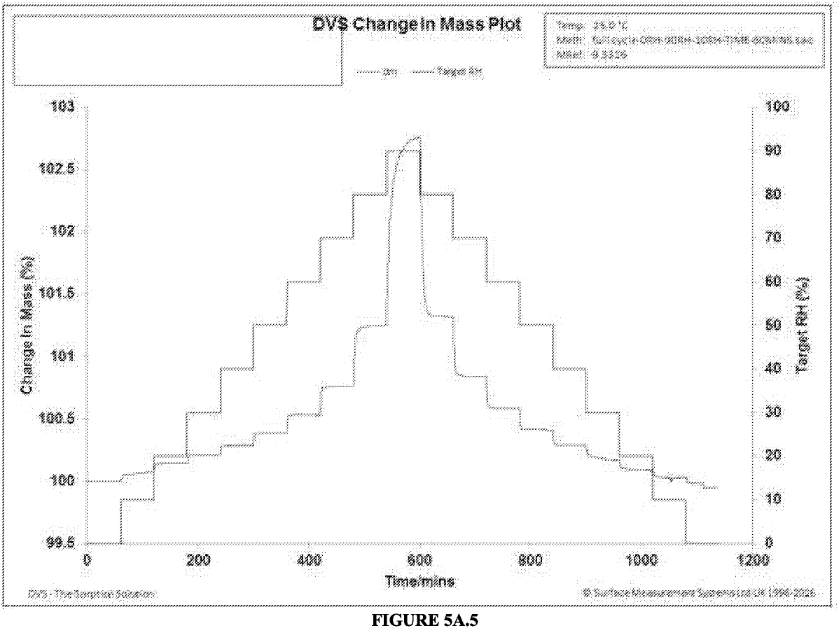

In some embodiments, Form A of compound 5 is characterized in that it has each of the spectral peaks in its X-ray powder diffraction pattern listed in Table 6 having a relative intensity greater than 10%, 20%, 30% or 40%. In certain embodiments, the X-ray powder diffraction pattern is substantially similar to the XRPD provided in FIG. 5A.1.

Methods for preparing Form A of compound 5 are described infra.

In some embodiments, the disclosure provides compound 5:

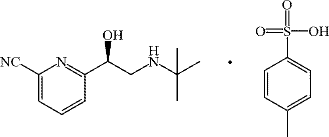

wherein said compound is crystalline. In some embodiments, the present disclosure provides compound 5, wherein said compound is substantially free of amorphous compound 5.

In some embodiments, the present disclosure provides compound 5, wherein said compound is substantially free of impurities.

In some embodiments, the present disclosure provides compound 5, wherein said compound has one or more peaks in its XRPD selected from those at about 7.1, about 7.6, about 15.4, about 19.9, and about 23.3 degrees 2-theta. In some such embodiments, the present disclosure provides compound 5, wherein said compound has at least two peaks in its XRPD selected from those at about 7.1, about 7.6, about 15.4, about 19.9, and about 23.3 degrees 2-theta. In some such embodiments, the present disclosure provides compound 5, wherein said compound is of Form A.

In some embodiments, the present disclosure provides compound 5, wherein said compound has an XRPD substantially similar to that depicted in FIG. 5A.1.

In some embodiments, the present disclosure provides a composition comprising compound 5 and a pharmaceutically acceptable carrier or excipient.

In some embodiments, the present disclosure provides a method of activating an adrenergic receptor in a patient comprising administering to said patient compound 5 or a composition thereof. In some embodiments, the adrenergic receptor is selected from β1-adrenergic receptor and β2-adrenergic receptor.

In some embodiments, the present disclosure provides a method of treating a β1-adrenergic receptor or β2-adrenergic receptor mediated disease or disorder in a patient, comprising administering to said patient compound 5 or composition thereof.

### Compound 6 (Maleate Salts of Compound 1)

According to one embodiment, the present disclosure provides a maleate salt of compound 1, represented by compound 6:

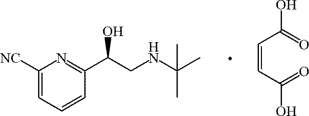

It will be appreciated by one of ordinary skill in the art that the maleic acid and compound 1 are ionically bonded to form compound 6. It is contemplated that compound 6 can exist in a variety of physical forms. For example, compound 6 can be in solution, suspension, or in solid form. In certain embodiments, compound 6 is in solid form. When compound 6 is in solid form, said compound may be amorphous, crystalline, or a mixture thereof. Exemplary solid forms are described in more detail below.

In some embodiments, the present disclosure provides a form of compound 6 substantially free of impurities. As used herein, the term “substantially free of impurities” means that the compound contains no significant amount of extraneous matter. Such extraneous matter may include different forms of compound 6, residual solvents, or any other impurities that may result from the preparation of, and/or isolation of, compound 6. In certain embodiments, at least about 95% by weight of a form of compound 6 is present. In still other embodiments of the disclosure, at least about 99% by weight of a form of compound 6 is present.

According to one embodiment, a form of compound 6 is present in an amount of at least about 97, 97.5, 98.0, 98.5, 99, 99.5, 99.8 weight percent where the percentages are based on the total weight of the composition. According to another embodiment, a form of compound 6 contains no more than about 3.0 area percent HPLC of total organic impurities and, in certain embodiments, no more than about 1.5 area percent HPLC total organic impurities relative to the total area of the HPLC chromatogram. In other embodiments, a form of compound 6 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 6 is also meant to include all tautomeric forms of compound 6. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 6 can exist in a variety of solid forms. Exemplary such forms include polymorphs such as those described herein.

In certain embodiments, compound 6 is a crystalline solid. In other embodiments, compound 6 is a crystalline solid substantially free of amorphous compound 6. As used herein, the term “substantially free of amorphous compound 6” means that the compound contains no significant amount of amorphous compound 6. In certain embodiments, at least about 95% by weight of crystalline compound 6 is present. In still other embodiments of the disclosure, at least about 99% by weight of crystalline compound 6 is present.

It has been found that compound 6 can exist in at least one distinct polymorphic form. In certain embodiments, the present disclosure provides a polymorphic form of compound 6 referred to herein as Form A.

In some embodiments, compound 6 is amorphous. In some embodiments, compound 6 is amorphous, and is substantially free of crystalline compound 6.

### Form A of Compound 6

In some embodiments, Form A of compound 6 has at least 1, 2, 3, 4 or 5 spectral peak(s) selected from the peaks listed in Table 7 below.

TABLE 7

XRPD Peak Positions for Form A of Compound 6

| Position (°2θ) | Intensity % | Position (°2θ) | Intensity % |
|---|---|---|---|
| 6.4 | 2.7 | 24.7 | 2.8 |
| 7.7 | 100.0 | 25.6 | 4.4 |
| 8.6 | 22.1 | 26.0 | 28.2 |
| 10.2 | 4.2 | 26.3 | 17.2 |
| 10.8 | 40.7 | 27.4 | 20.2 |
| 12.9 | 7.2 | 29.1 | 5.4 |
| 14.0 | 1.4 | 30.0 | 3.4 |
| 14.5 | 12.9 | 30.8 | 3.0 |
| 15.1 | 4.6 | 31.1 | 4.8 |
| 15.5 | 7.5 | 32.8 | 2.3 |
| 16.4 | 5.0 | 33.2 | 2.0 |
| 17.4 | 5.7 | 33.8 | 2.2 |
| 18.0 | 19.5 | 34.1 | 3.4 |
| 18.5 | 9.1 | 34.7 | 2.5 |
| 19.5 | 2.2 | 35.3 | 5.7 |
| 19.9 | 16.1 | 36.1 | 1.6 |
| 20.5 | 4.2 | 36.6 | 2.0 |
| 21.3 | 2.7 | 37.2 | 1.6 |
| 22.2 | 2.1 | 37.8 | 1.1 |
| 22.7 | 8.4 | 38.3 | 2.3 |
| 23.4 | 16.6 | 38.8 | 1.9 |
| 24.3 | 5.4 | 39.6 | 2.0 |

In this and all subsequent tables, the position (°2θ) is within ± 0.2.

In some embodiments, Form A of compound 6 is characterized in that it has one or more peaks in its X-ray powder diffraction pattern selected from those at about 7.7, about 8.6, about 10.8, about 26.0, and about 27.4 degrees 2-theta. In some embodiments, Form A of compound 6 is characterized in that it has two or more peaks in its X-ray powder diffraction pattern selected from those at about 7.7, about 8.6, about 10.8, about 26.0, and about 27.4 degrees 2-theta. In some embodiments, Form A of compound 6 is characterized in that it has three or more peaks in its X-ray powder diffraction pattern selected from those at about 7.7, about 8.6, about 10.8, about 26.0, and about 27.4 degrees 2-theta. In some embodiments, Form A of compound 6 is characterized in that it has four or more peaks in its X-ray powder diffraction pattern selected from those at about 7.7, about 8.6, about 10.8, about 26.0, and about 27.4 degrees 2-theta. In some embodiments, Form A of compound 6 is characterized in that it has five peaks in its X-ray powder diffraction pattern selected from those at about 7.7, about 8.6, about 10.8, about 26.0, and about 27.4 degrees 2-theta.

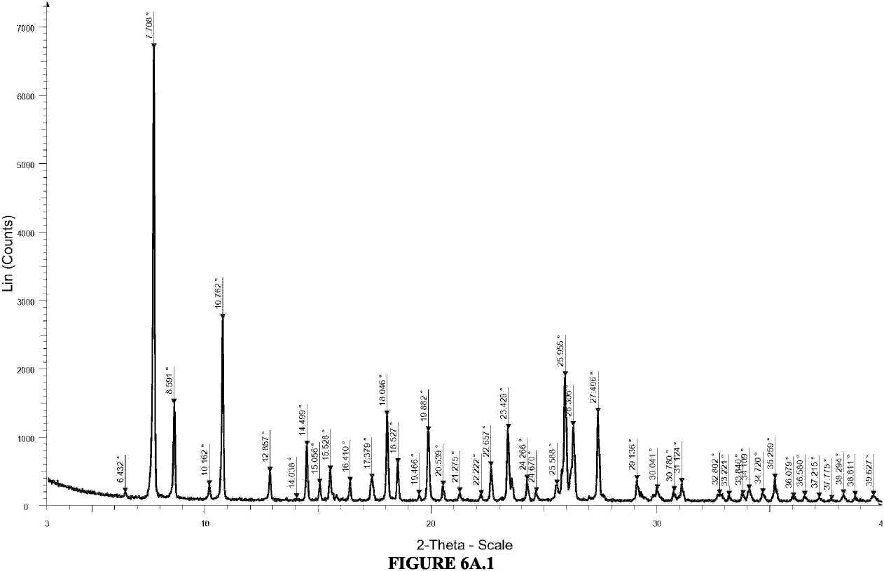

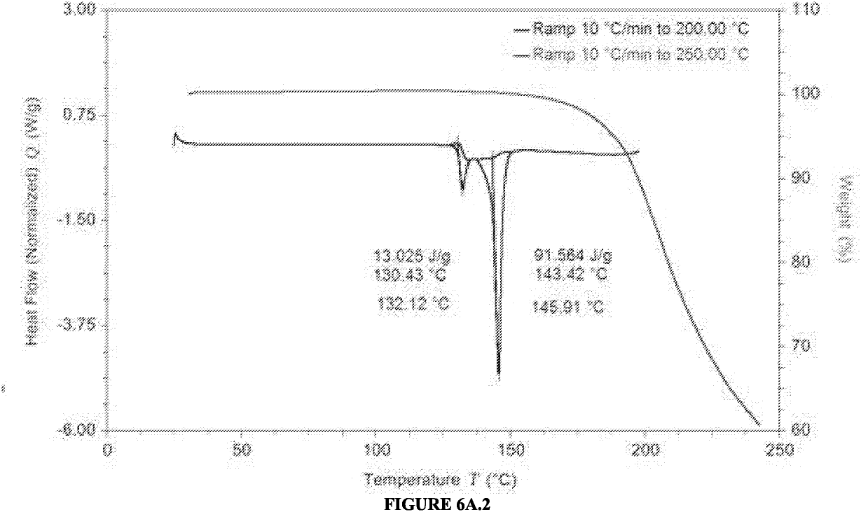

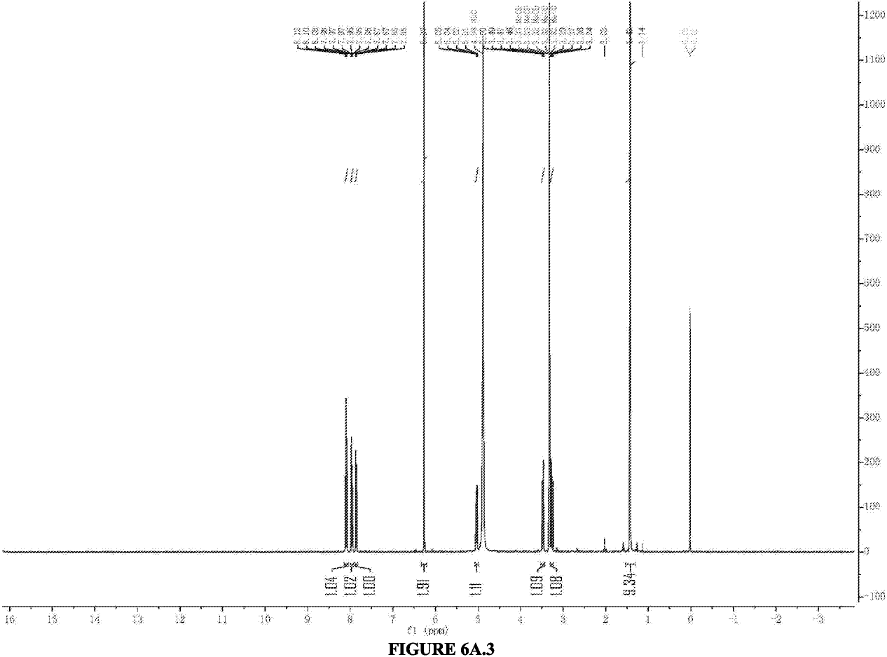

In some embodiments, Form A of compound 6 is characterized in that it has each of the spectral peaks in its X-ray powder diffraction pattern listed in Table 7 having a relative intensity greater than 10%, 20%, 30% or 40%. In certain embodiments, the X-ray powder diffraction pattern is substantially similar to the XRPD provided in FIG. 6A.1.

Methods for preparing Form A of compound 6 are described infra.

In some embodiments, the disclosure provides compound 6:

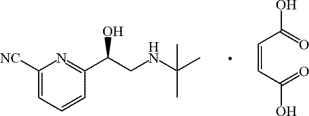

wherein said compound is crystalline. In some embodiments, the present disclosure provides compound 6, wherein said compound is substantially free of amorphous compound 6.

In some embodiments, the present disclosure provides compound 6, wherein said compound is substantially free of impurities.

In some embodiments, the present disclosure provides compound 6, wherein said compound has one or more peaks in its XRPD selected from those at about 7.7, about 8.6, about 10.8, about 26.0, and about 27.4 degrees 2-theta. In some such embodiments, the present disclosure provides compound 6, wherein said compound has at least two peaks in its XRPD selected from those at about 7.7, about 8.6, about 10.8, about 26.0, and about 27.4 degrees 2-theta. In some such embodiments, the present disclosure provides compound 6, wherein said compound is of Form A.

In some embodiments, the present disclosure provides compound 6, wherein said compound has an XRPD substantially similar to that depicted in FIG. 6A.1.

In some embodiments, the present disclosure provides a composition comprising compound 6 and a pharmaceutically acceptable carrier or excipient.

In some embodiments, the present disclosure provides a method of activating an adrenergic receptor in a patient comprising administering to said patient compound 6 or a composition thereof. In some embodiments, the adrenergic receptor is selected from β1-adrenergic receptor and β2-adrenergic receptor.

In some embodiments, the present disclosure provides a method of treating a β1-adrenergic receptor or β2-adrenergic receptor mediated disease or disorder in a patient, comprising administering to said patient compound 6 or composition thereof.

### Compound 7 (Fumarate Salts of Compound 1)

According to one embodiment, the present disclosure provides a fumarate salt of compound 1, represented by compound 7:

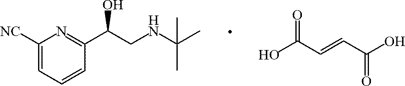

It will be appreciated by one of ordinary skill in the art that the fumaric acid and compound 1 are ionically bonded to form compound 7. It is contemplated that compound 7 can exist in a variety of physical forms. For example, compound 7 can be in solution, suspension, or in solid form. In certain embodiments, compound 7 is in solid form. When compound 7 is in solid form, said compound may be amorphous, crystalline, or a mixture thereof. Exemplary solid forms are described in more detail below.

In some embodiments, the present disclosure provides a form of compound 7 substantially free of impurities. As used herein, the term “substantially free of impurities” means that the compound contains no significant amount of extraneous matter. Such extraneous matter may include different forms of compound 7, residual solvents, or any other impurities that may result from the preparation of, and/or isolation of, compound 7. In certain embodiments, at least about 95% by weight of a form of compound 7 is present. In still other embodiments of the disclosure, at least about 99% by weight of a form of compound 7 is present.

According to one embodiment, a form of compound 7 is present in an amount of at least about 97, 97.5, 98.0, 98.5, 99, 99.5, 99.8 weight percent where the percentages are based on the total weight of the composition. According to another embodiment, a form of compound 7 contains no more than about 3.0 area percent HPLC of total organic impurities and, in certain embodiments, no more than about 1.5 area percent HPLC total organic impurities relative to the total area of the HPLC chromatogram. In other embodiments, a form of compound 7 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 7 is also meant to include all tautomeric forms of compound 7. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 7 can exist in a variety of solid forms. Exemplary such forms include polymorphs such as those described herein.

In certain embodiments, compound 7 is a crystalline solid. In other embodiments, compound 7 is a crystalline solid substantially free of amorphous compound 7. As used herein, the term “substantially free of amorphous compound 7” means that the compound contains no significant amount of amorphous compound 7. In certain embodiments, at least about 95% by weight of crystalline compound 7 is present. In still other embodiments of the disclosure, at least about 99% by weight of crystalline compound 7 is present.

It has been found that compound 7 can exist in at least one distinct polymorphic form. In certain embodiments, the present disclosure provides a polymorphic form of compound 7 referred to herein as Form A.

In some embodiments, compound 7 is amorphous. In some embodiments, compound 7 is amorphous, and is substantially free of crystalline compound 7.

### Form A of Compound 7

In some embodiments, Form A of compound 7 has at least 1, 2, 3, 4 or 5 spectral peak(s) selected from the peaks listed in Table 8 below.

TABLE 8

XRPD Peak Positions for Form A of Compound 7

| Position (°2θ) | Intensity % | Position (°2θ) | Intensity % |
|---|---|---|---|
| 4.9 | 11.3 | 17.5 | 11.3 |
| 5.5 | 14.7 | 18.3 | 4.1 |
| 5.8 | 12.0 | 19.5 | 6.4 |
| 7.4 | 15.5 | 21.4 | 4.3 |
| 10.1 | 15.7 | 22.6 | 12.1 |
| 11.1 | 100.0 | 23.0 | 34.5 |
| 12.2 | 6.3 | 23.9 | 8.4 |
| 12.8 | 4.7 | 24.6 | 43.3 |
| 14.0 | 5.5 | 26.4 | 13.3 |
| 15.0 | 6.6 | 27.1 | 11.3 |
| 15.7 | 6.4 | 28.2 | 11.7 |
| 16.0 | 7.1 | 30.8 | 4.7 |
| 16.7 | 5.1 | 35.5 | 3.1 |

In this and all subsequent tables, the position (°2θ) is within ± 0.2.

In some embodiments, Form A of compound 7 is characterized in that it has one or more peaks in its X-ray powder diffraction pattern selected from those at about 7.4, about 10.1, about 11.1, about 23.0, and about 24.6 degrees 2-theta. In some embodiments, Form A of compound 7 is characterized in that it has two or more peaks in its X-ray powder diffraction pattern selected from those at about 7.4, about 10.1, about 11.1, about 23.0, and about 24.6 degrees 2-theta. In some embodiments, Form A of compound 7 is characterized in that it has three or more peaks in its X-ray powder diffraction pattern selected from those at about 7.4, about 10.1, about 11.1, about 23.0, and about 24.6 degrees 2-theta. In some embodiments, Form A of compound 7 is characterized in that it has four or more peaks in its X-ray powder diffraction pattern selected from those at about 7.4, about 10.1, about 11.1, about 23.0, and about 24.6 degrees 2-theta. In some embodiments, Form A of compound 7 is characterized in that it has five peaks in its X-ray powder diffraction pattern selected from those at about 7.4, about 10.1, about 11.1, about 23.0, and about 24.6 degrees 2-theta.

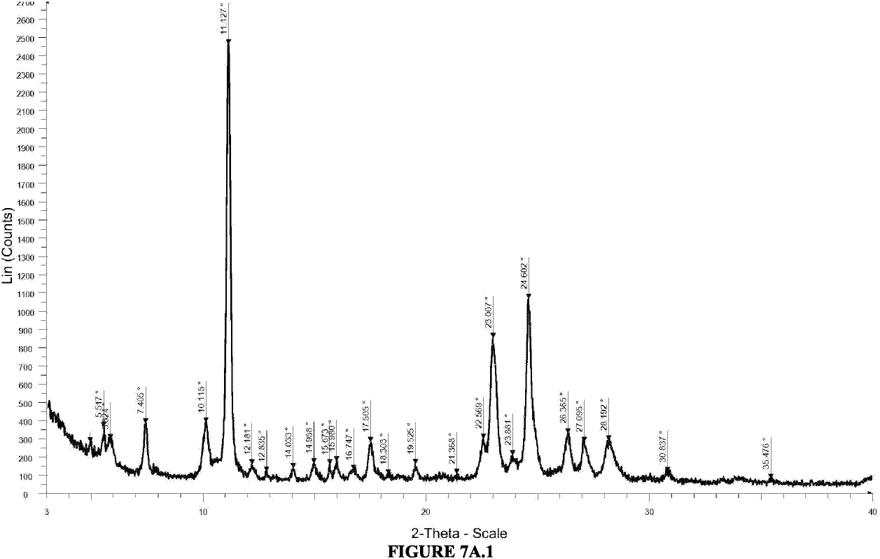

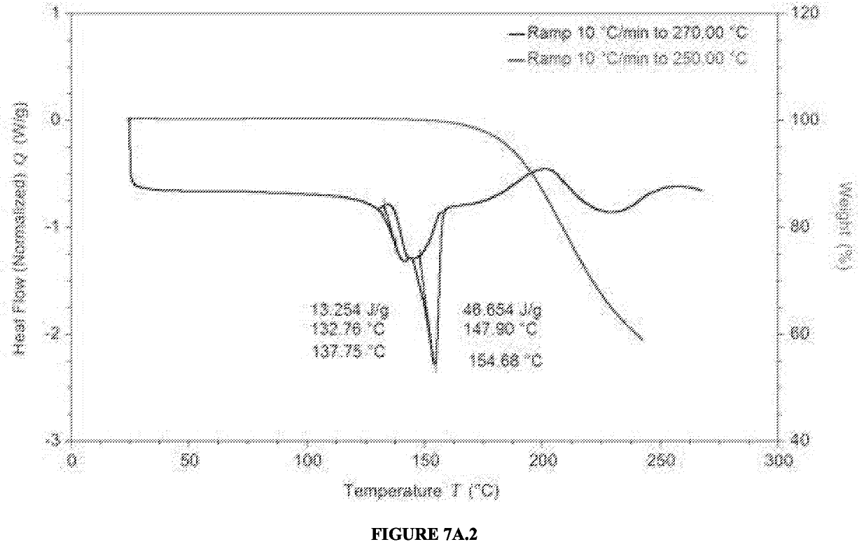

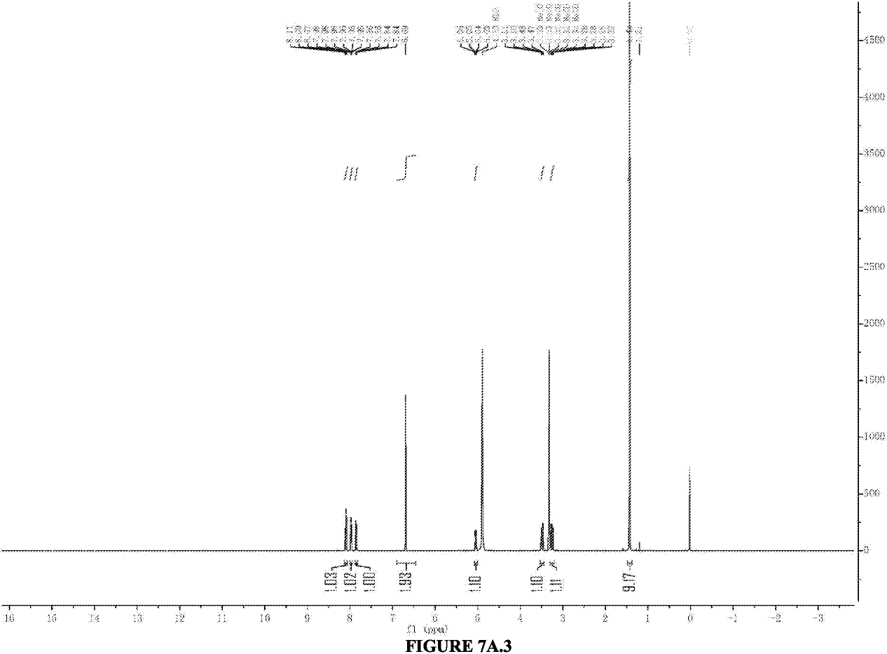

In some embodiments, Form A of compound 7 is characterized in that it has each of the spectral peaks in its X-ray powder diffraction pattern listed in Table 8 having a relative intensity greater than 10%, 20%, 30% or 40%. In certain embodiments, the X-ray powder diffraction pattern is substantially similar to the XRPD provided in FIG. 7A.1.

Methods for preparing Form A of compound 7 are described infra.

In some embodiments, the disclosure provides compound 7:

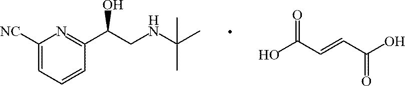

wherein said compound is crystalline. In some embodiments, the present disclosure provides compound 7, wherein said compound is substantially free of amorphous compound 7.

In some embodiments, the present disclosure provides compound 7, wherein said compound is substantially free of impurities.

In some embodiments, the present disclosure provides compound 7, wherein said compound has one or more peaks in its XRPD selected from those at about 7.4, about 10.1, about 11.1, about 23.0, and about 24.6 degrees 2-theta. In some such embodiments, the present disclosure provides compound 7, wherein said compound has at least two peaks in its XRPD selected from those at about 7.4, about 10.1, about 11.1, about 23.0, and about 24.6 degrees 2-theta. In some such embodiments, the present disclosure provides compound 7, wherein said compound is of Form A.

In some embodiments, the present disclosure provides compound 7, wherein said compound has an XRPD substantially similar to that depicted in FIG. 7A.1.

In some embodiments, the present disclosure provides a composition comprising compound 7 and a pharmaceutically acceptable carrier or excipient.

In some embodiments, the present disclosure provides a method of activating an adrenergic receptor in a patient comprising administering to said patient compound 7 or a composition thereof. In some embodiments, the adrenergic receptor is selected from β1-adrenergic receptor and β2-adrenergic receptor.

In some embodiments, the present disclosure provides a method of treating a β1-adrenergic receptor or β2-adrenergic receptor mediated disease or disorder in a patient, comprising administering to said patient compound 7 or composition thereof.

### Compound 8 (Glycolate Salts of Compound 1)

According to one embodiment, the present disclosure provides a glycolate salt of compound 1, represented by compound 8:

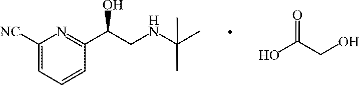

It will be appreciated by one of ordinary skill in the art that the glycolic acid and compound 1 are ionically bonded to form compound 8. It is contemplated that compound 8 can exist in a variety of physical forms. For example, compound 8 can be in solution, suspension, or in solid form. In certain embodiments, compound 8 is in solid form. When compound 8 is in solid form, said compound may be amorphous, crystalline, or a mixture thereof. Exemplary solid forms are described in more detail below.

In some embodiments, the present disclosure provides a form of compound 8 substantially free of impurities. As used herein, the term “substantially free of impurities” means that the compound contains no significant amount of extraneous matter. Such extraneous matter may include different forms of compound 8, residual solvents, or any other impurities that may result from the preparation of, and/or isolation of, compound 8. In certain embodiments, at least about 95% by weight of a form of compound 8 is present. In still other embodiments of the disclosure, at least about 99% by weight of a form of compound 8 is present.

According to one embodiment, a form of compound 8 is present in an amount of at least about 97, 97.5, 98.0, 98.5, 99, 99.5, 99.8 weight percent where the percentages are based on the total weight of the composition. According to another embodiment, a form of compound 8 contains no more than about 3.0 area percent HPLC of total organic impurities and, in certain embodiments, no more than about 1.5 area percent HPLC total organic impurities relative to the total area of the HPLC chromatogram. In other embodiments, a form of compound 8 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 8 is also meant to include all tautomeric forms of compound 8. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 8 can exist in a variety of solid forms. Exemplary such forms include polymorphs such as those described herein.

In certain embodiments, compound 8 is a crystalline solid. In other embodiments, compound 8 is a crystalline solid substantially free of amorphous compound 8. As used herein, the term “substantially free of amorphous compound 8” means that the compound contains no significant amount of amorphous compound 8. In certain embodiments, at least about 95% by weight of crystalline compound 8 is present. In still other embodiments of the disclosure, at least about 99% by weight of crystalline compound 8 is present.

It has been found that compound 8 can exist in at least one distinct polymorphic form. In certain embodiments, the present disclosure provides a polymorphic form of compound 8 referred to herein as Form A.

In some embodiments, compound 8 is amorphous. In some embodiments, compound 8 is amorphous, and is substantially free of crystalline compound 8.

### Form A of Compound 8

In some embodiments, Form A of compound 8 has at least 1, 2, 3, 4 or 5 spectral peak(s) selected from the peaks listed in Table 9 below.

TABLE 9

XRPD Peak Positions for Form A of Compound 8

| Position (°2θ) | Intensity % | Position (°2θ) | Intensity % |
|---|---|---|---|
| 3.7 | 25.0 | 24.3 | 19.6 |
| 5.7 | 55.2 | 25.1 | 13.3 |
| 6.4 | 11.3 | 25.5 | 13.9 |
| 10.1 | 14.1 | 25.8 | 19.3 |
| 10.9 | 19.9 | 26.3 | 5.9 |
| 11.6 | 68.6 | 27.2 | 7.1 |
| 12.0 | 20.3 | 27.6 | 5.7 |
| 15.4 | 12.9 | 29.6 | 13.1 |
| 15.7 | 100.0 | 31.4 | 5.3 |
| 16.7 | 12.5 | 31.8 | 6.0 |
| 17.5 | 68.4 | 32.6 | 6.9 |
| 18.4 | 12.1 | 32.9 | 8.3 |
| 18.7 | 8.4 | 34.2 | 5.7 |
| 19.8 | 12.6 | 35.1 | 4.4 |
| 20.4 | 40.4 | 37.2 | 5.7 |
| 22.2 | 8.9 | 38.5 | 4.8 |
| 22.7 | 8.0 | 39.5 | 7.8 |
| 23.1 | 70.5 | | |

In this and all subsequent tables, the position (°2θ) is within ± 0.2.

In some embodiments, Form A of compound 8 is characterized in that it has one or more peaks in its X-ray powder diffraction pattern selected from those at about 5.7, about 11.6, about 15.7, about 17.5, about 20.4, and about 23.1 degrees 2-theta. In some embodiments, Form A of compound 8 is characterized in that it has two or more peaks in its X-ray powder diffraction pattern selected from those at about 5.7, about 11.6, about 15.7, about 17.5, about 20.4, and about 23.1 degrees 2-theta. In some embodiments, Form A of compound 8 is characterized in that it has three or more peaks in its X-ray powder diffraction pattern selected from those at about 5.7, about 11.6, about 15.7, about 17.5, about 20.4, and about 23.1 degrees 2-theta. In some embodiments, Form A of compound 8 is characterized in that it has four or more peaks in its X-ray powder diffraction pattern selected from those at about 5.7, about 11.6, about 15.7, about 17.5, about 20.4, and about 23.1 degrees 2-theta. In some embodiments, Form A of compound 8 is characterized in that it has five or more peaks in its X-ray powder diffraction pattern selected from those at about 5.7, about 11.6, about 15.7, about 17.5, about 20.4, and about 23.1 degrees 2-theta. In some embodiments, Form A of compound 8 is characterized in that it has six peaks in its X-ray powder diffraction pattern selected from those at about 5.7, about 11.6, about 15.7, about 17.5, about 20.4, and about 23.1 degrees 2-theta.

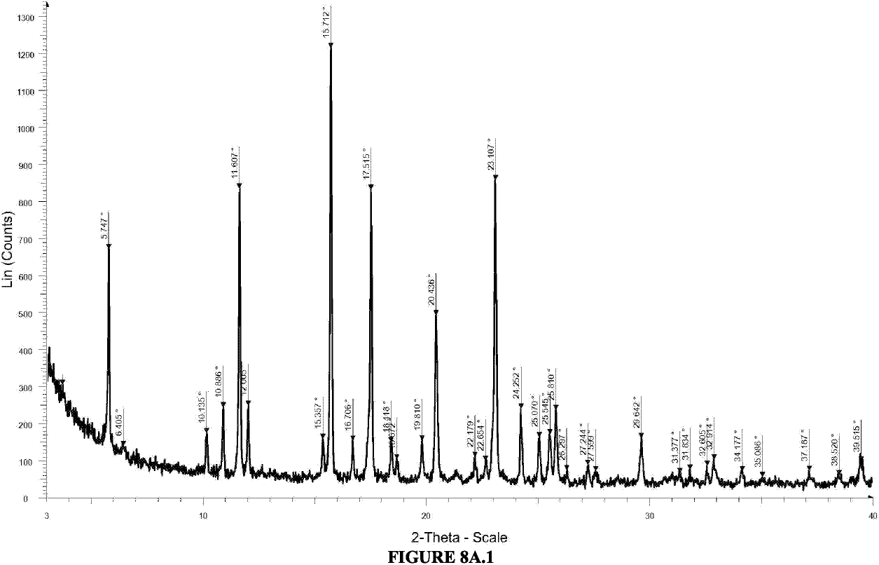

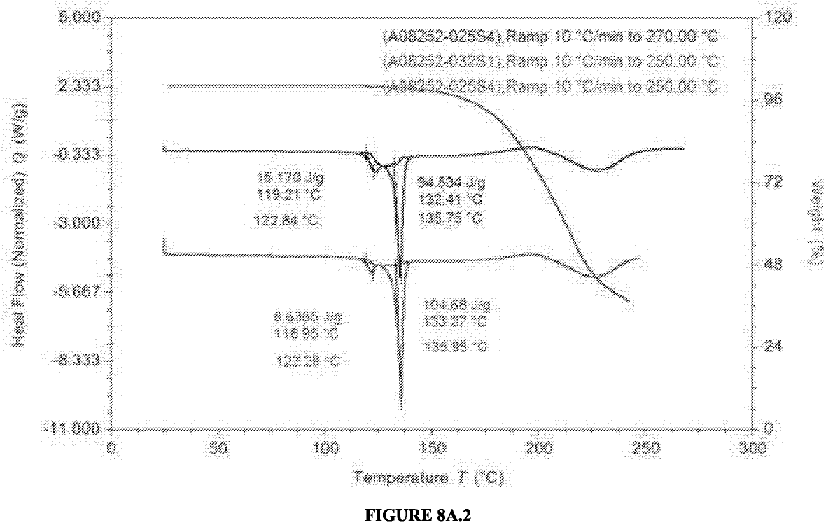

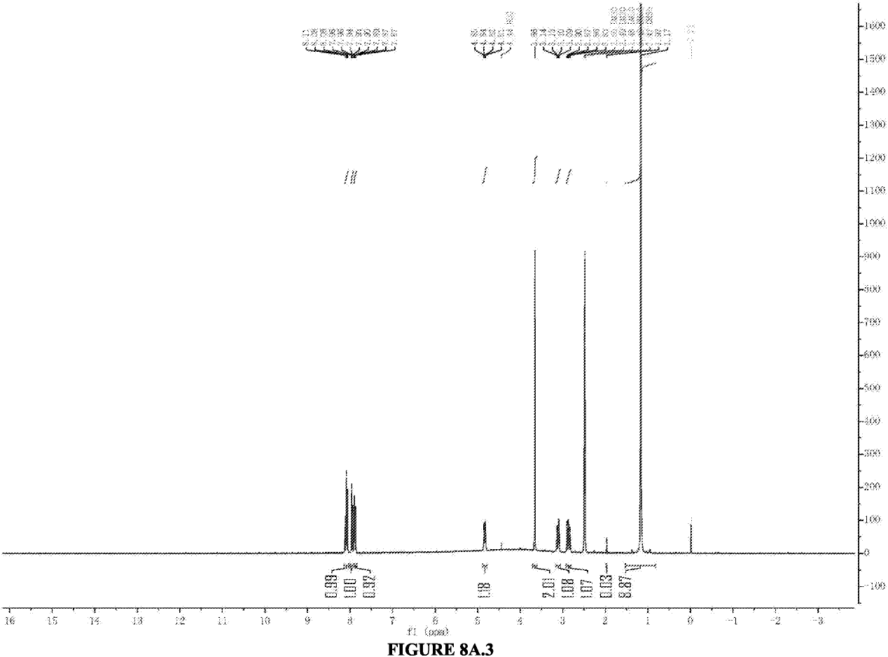

In some embodiments, Form A of compound 8 is characterized in that it has each of the spectral peaks in its X-ray powder diffraction pattern listed in Table 9 having a relative intensity greater than 10%, 20%, 30% or 40%. In certain embodiments, the X-ray powder diffraction pattern is substantially similar to the XRPD provided in FIG. 8A.1.

Methods for preparing Form A of compound 8 are described infra.

In some embodiments, the disclosure provides compound 8:

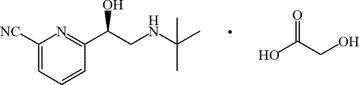

wherein said compound is crystalline. In some embodiments, the present disclosure provides compound 8, wherein said compound is substantially free of amorphous compound 8.

In some embodiments, the present disclosure provides compound 8, wherein said compound is substantially free of impurities.

In some embodiments, the present disclosure provides compound 8, wherein said compound has one or more peaks in its XRPD selected from those at about 5.7, about 11.6, about 15.7, about 17.5, about 20.4, and about 23.1 degrees 2-theta. In some such embodiments, the present disclosure provides compound 8, wherein said compound has at least two peaks in its XRPD selected from those at about 5.7, about 11.6, about 15.7, about 17.5, about 20.4, and about 23.1 degrees 2-theta. In some such embodiments, the present disclosure provides compound 8, wherein said compound is of Form A.

In some embodiments, the present disclosure provides compound 8, wherein said compound has an XRPD substantially similar to that depicted in FIG. 8A.1.

In some embodiments, the present disclosure provides a composition comprising compound 8 and a pharmaceutically acceptable carrier or excipient.

In some embodiments, the present disclosure provides a method of activating an adrenergic receptor in a patient comprising administering to said patient compound 8 or a composition thereof. In some embodiments, the adrenergic receptor is selected from β1-adrenergic receptor and β2-adrenergic receptor.

In some embodiments, the present disclosure provides a method of treating a β1-adrenergic receptor or β2-adrenergic receptor mediated disease or disorder in a patient, comprising administering to said patient compound 8 or composition thereof.

### Compound 9 (L-tartrate Salts of Compound 1)

According to one embodiment, the present disclosure provides an L-tartrate salt of compound 1, represented by compound 9:

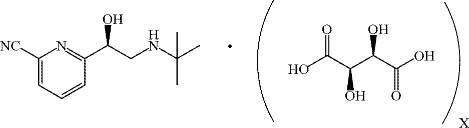

wherein 0 < X ≤ 1.

It will be appreciated by one of ordinary skill in the art that the L-(+)-tartaric acid and compound 1 are ionically bonded to form compound 9. It is contemplated that compound 9 can exist in a variety of physical forms. For example, compound 9 can be in solution, suspension, or in solid form. In certain embodiments, compound 9 is in solid form. When compound 9 is in solid form, said compound may be amorphous, crystalline, or a mixture thereof. Exemplary solid forms are described in more detail below.

In some embodiments, the present disclosure provides a form of compound 9 substantially free of impurities. As used herein, the term “substantially free of impurities” means that the compound contains no significant amount of extraneous matter. Such extraneous matter may include different forms of compound 9, residual solvents, or any other impurities that may result from the preparation of, and/or isolation of, compound 9. In certain embodiments, at least about 95% by weight of a form of compound 9 is present. In still other embodiments of the disclosure, at least about 99% by weight of a form of compound 9 is present.

According to one embodiment, a form of compound 9 is present in an amount of at least about 97, 97.5, 98.0, 98.5, 99, 99.5, 99.8 weight percent where the percentages are based on the total weight of the composition. According to another embodiment, a form of compound 9 contains no more than about 3.0 area percent HPLC of total organic impurities and, in certain embodiments, no more than about 1.5 area percent HPLC total organic impurities relative to the total area of the HPLC chromatogram. In other embodiments, a form of compound 9 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 9 is also meant to include all tautomeric forms of compound 9. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 9 can exist in a variety of solid forms. Exemplary such forms include polymorphs such as those described herein.

In certain embodiments, compound 9 is a crystalline solid. In other embodiments, compound 9 is a crystalline solid substantially free of amorphous compound 9. As used herein, the term “substantially free of amorphous compound 9” means that the compound contains no significant amount of amorphous compound 9. In certain embodiments, at least about 95% by weight of crystalline compound 9 is present. In still other embodiments of the disclosure, at least about 99% by weight of crystalline compound 9 is present.

It has been found that compound 9 can exist in at least one distinct polymorphic form. In certain embodiments, the present disclosure provides a polymorphic form of compound 9 referred to herein as Form A.

In some embodiments, compound 9 is amorphous. In some embodiments, compound 9 is amorphous, and is substantially free of crystalline compound 9.

In some embodiments, X is 0.5

### Form A of Compound 9

In some embodiments, Form A of compound 9 has at least 1, 2, 3, 4 or 5 spectral peak(s) selected from the peaks listed in Table 10 below.

TABLE 10

XRPD Peak Positions for Form A of Compound 9

| Position (°2θ) | Intensity % | Position (°2θ) | Intensity % |
|---|---|---|---|
| 3.7 | 16.8 | 20.0 | 7.1 |
| 5.6 | 12.4 | 20.7 | 4.6 |
| 10.9 | 14.7 | 21.2 | 3.4 |
| 11.2 | 100.0 | 22.0 | 27.7 |
| 12.4 | 6.8 | 22.5 | 21.8 |
| 13.6 | 8.8 | 23.3 | 8.4 |
| 14.2 | 5.5 | 24.5 | 11.2 |
| 16.0 | 10.3 | 27.2 | 4.5 |
| 16.8 | 6.3 | 28.5 | 2.9 |
| 17.1 | 6.7 | 32.3 | 2.7 |
| 18.4 | 10.0 | 33.5 | 4.3 |

In this and all subsequent tables, the position (°2θ) is within ± 0.2.

In some embodiments, Form A of compound 9 is a salt wherein X is 0.5.

In some embodiments, Form A of compound 9 is characterized in that it has one or more peaks in its X-ray powder diffraction pattern selected from those at about 11.2, about 22.0, and about 22.5 degrees 2-theta. In some embodiments, Form A of compound 9 is characterized in that it has two or more peaks in its X-ray powder diffraction pattern selected from those at about 11.2, about 22.0, and about 22.5 degrees 2-theta. In some embodiments, Form A of compound 9 is characterized in that it has three peaks in its X-ray powder diffraction pattern selected from those at about 11.2, about 22.0, and about 22.5 degrees 2-theta.

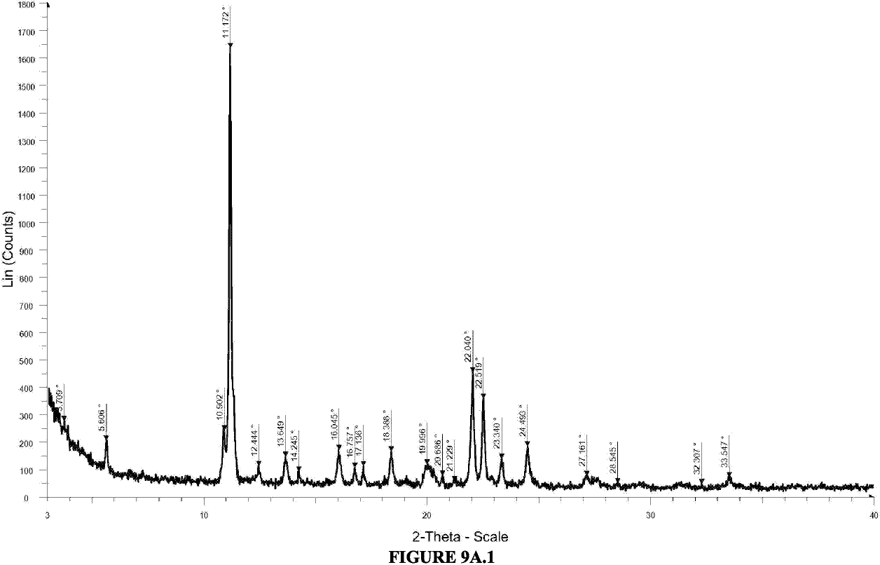

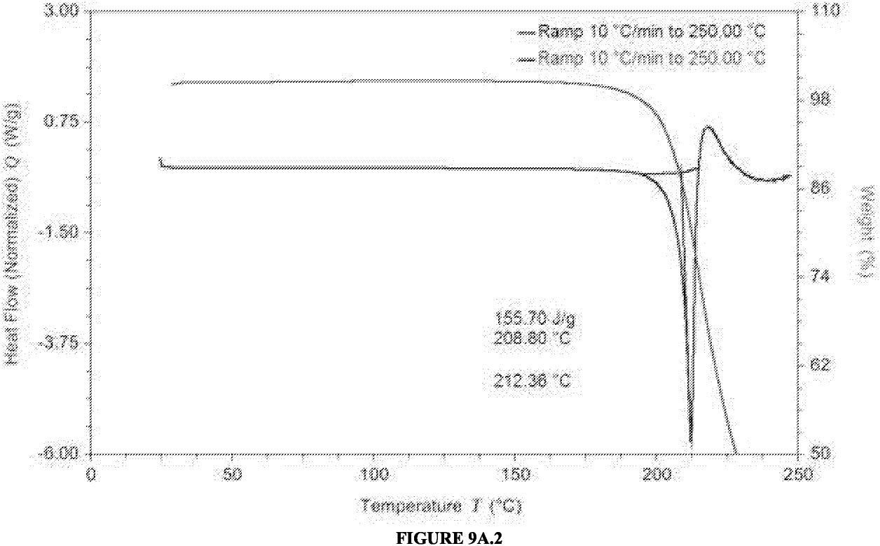

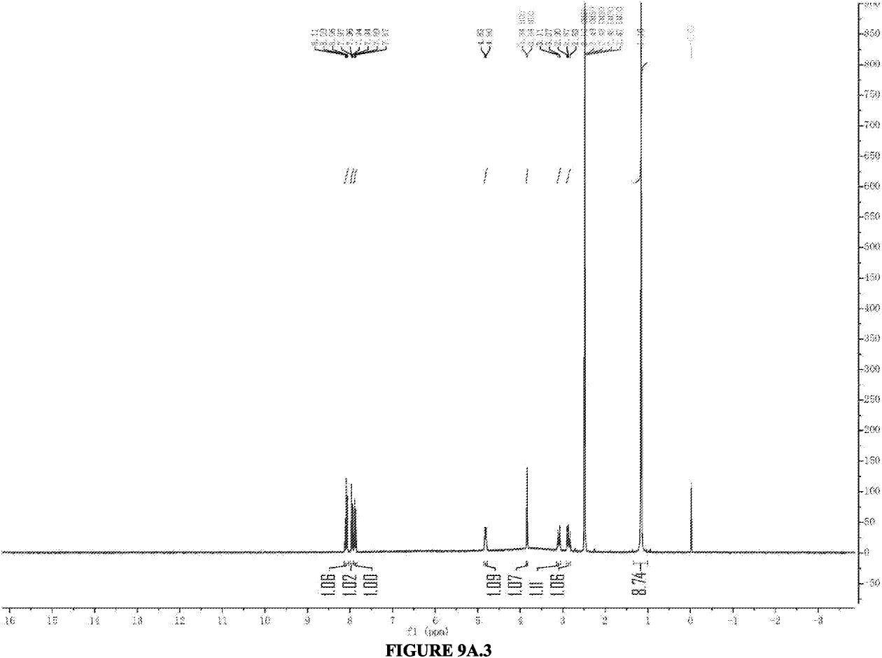

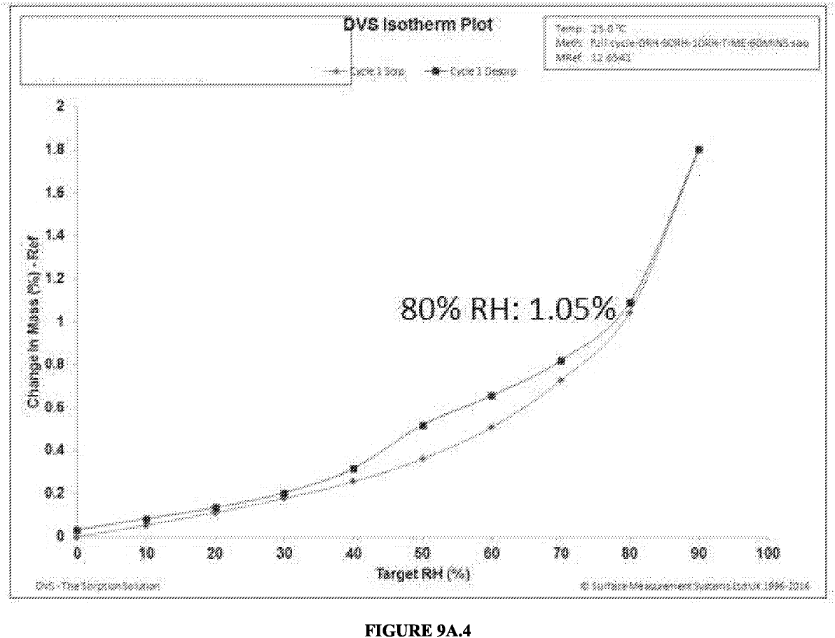

In some embodiments, Form A of compound 9 is characterized in that it has each of the spectral peaks in its X-ray powder diffraction pattern listed in Table 10 having a relative intensity greater than 10%, 20%, 30% or 40%. In certain embodiments, the X-ray powder diffraction pattern is substantially similar to the XRPD provided in FIG. 9A.1.

Methods for preparing Form A of compound 9 are described infra.

In some embodiments, the disclosure provides compound 9:

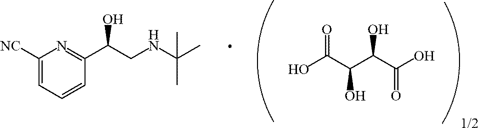

wherein said compound is crystalline. In some embodiments, the present disclosure provides compound 9, wherein said compound is substantially free of amorphous compound 9.

In some embodiments, the present disclosure provides compound 9, wherein said compound is substantially free of impurities.

In some embodiments, the present disclosure provides compound 9, wherein said compound has one or more peaks in its XRPD selected from those at about 11.2, about 22.0, and about 22.5 degrees 2-theta. In some such embodiments, the present disclosure provides compound 9, wherein said compound has at least two peaks in its XRPD selected from those at about 11.2, about 22.0, and about 22.5 degrees 2-theta. In some such embodiments, the present disclosure provides compound 9, wherein said compound is of Form A.

In some embodiments, the present disclosure provides compound 9, wherein said compound has an XRPD substantially similar to that depicted in FIG. 9A.1.

In some embodiments, the present disclosure provides a composition comprising compound 9 and a pharmaceutically acceptable carrier or excipient.

In some embodiments, the present disclosure provides a method of activating an adrenergic receptor in a patient comprising administering to said patient compound 9 or a composition thereof. In some embodiments, the adrenergic receptor is selected from β1-adrenergic receptor and β2-adrenergic receptor.

In some embodiments, the present disclosure provides a method of treating a β1-adrenergic receptor or β2-adrenergic receptor mediated disease or disorder in a patient, comprising administering to said patient compound 9 or composition thereof.

### Compound 10 (L-malate Salts of Compound 1)

According to one embodiment, the present disclosure provides a L-malate salt of compound 1, represented by compound 10:

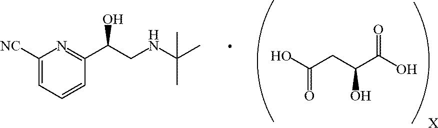

wherein 0 < X ≤ 1.

It will be appreciated by one of ordinary skill in the art that the L-malic acid and compound 1 are ionically bonded to form compound 10. It is contemplated that compound 10 can exist in a variety of physical forms. For example, compound 10 can be in solution, suspension, or in solid form. In certain embodiments, compound 10 is in solid form. When compound 10 is in solid form, said compound may be amorphous, crystalline, or a mixture thereof. Exemplary solid forms are described in more detail below.

In some embodiments, the present disclosure provides a form of compound 10 substantially free of impurities. As used herein, the term “substantially free of impurities” means that the compound contains no significant amount of extraneous matter. Such extraneous matter may include different forms of compound 10, residual solvents, or any other impurities that may result from the preparation of, and/or isolation of, compound 10. In certain embodiments, at least about 95% by weight of a form of compound 10 is present. In still other embodiments of the disclosure, at least about 99% by weight of a form of compound 10 is present.

According to one embodiment, a form of compound 10 is present in an amount of at least about 97, 97.5, 98.0, 98.5, 99, 99.5, 99.8 weight percent where the percentages are based on the total weight of the composition. According to another embodiment, a form of compound 10 contains no more than about 3.0 area percent HPLC of total organic impurities and, in certain embodiments, no more than about 1.5 area percent HPLC total organic impurities relative to the total area of the HPLC chromatogram. In other embodiments, a form of compound 10 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 10 is also meant to include all tautomeric forms of compound 10. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 10 can exist in a variety of solid forms. Exemplary such forms include polymorphs such as those described herein.

In certain embodiments, compound 10 is a crystalline solid. In other embodiments, compound 10 is a crystalline solid substantially free of amorphous compound 10. As used herein, the term “substantially free of amorphous compound 10” means that the compound contains no significant amount of amorphous compound 10. In certain embodiments, at least about 95% by weight of crystalline compound 10 is present. In still other embodiments of the disclosure, at least about 99% by weight of crystalline compound 10 is present.

It has been found that compound 10 can exist in at least one distinct polymorphic form. In certain embodiments, the present disclosure provides a polymorphic form of compound 10 referred to herein as Form A.

In some embodiments, compound 10 is amorphous. In some embodiments, compound 10 is amorphous, and is substantially free of crystalline compound 10.

In some embodiments, X is 0.5

### Form A of Compound 10

In some embodiments, Form A of compound 10 has at least 1, 2, 3, 4 or 5 spectral peak(s) selected from the peaks listed in Table 11 below.

TABLE 11

XRPD Peak Positions for Form A of Compound 10

| Position (°2θ) | Intensity % | Position (°2θ) | Intensity % |
|---|---|---|---|
| 5.7 | 44.9 | 21.6 | 29.5 |
| 10.3 | 100.0 | 22.0 | 16.4 |
| 10.8 | 41.3 | 22.4 | 34.9 |
| 11.1 | 16.9 | 23.1 | 22.5 |
| 11.5 | 36.4 | 23.7 | 75.8 |
| 11.7 | 63.5 | 24.6 | 17.5 |
| 12.7 | 13.8 | 25.3 | 53.5 |
| 14.4 | 39.3 | 26.6 | 35.6 |
| 15.0 | 47.3 | 27.9 | 9.6 |
| 16.5 | 64.4 | 29.2 | 10.7 |
| 17.5 | 15.3 | 29.9 | 9.8 |
| 18.8 | 13.5 | 31.5 | 12.4 |
| 19.6 | 39.8 | 32.1 | 11.1 |
| 20.3 | 21.6 | 32.9 | 15.3 |
| 21.3 | 32.9 | | |

In this and all subsequent tables, the position (°2θ) is within ± 0.2.

In some embodiments, Form A of compound 10 is a salt wherein X is 0.5.

In some embodiments, Form A of compound 10 is characterized in that it has one or more peaks in its X-ray powder diffraction pattern selected from those at about 10.3, about 10.8, about 11.7, about 15.0, about 16.5, about 23.7, about 25.3, and about 26.6 degrees 2-theta. In some embodiments, Form A of compound 10 is characterized in that it has two or more peaks in its X-ray powder diffraction pattern selected from those at about 10.3, about 10.8, about 11.7, about 15.0, about 16.5, about 23.7, about 25.3, and about 26.6 degrees 2-theta. In some embodiments, Form A of compound 10 is characterized in that it has three or more peaks in its X-ray powder diffraction pattern selected from those at about 10.3, about 10.8, about 11.7, about 15.0, about 16.5, about 23.7, about 25.3, and about 26.6 degrees 2-theta. In some embodiments, Form A of compound 10 is characterized in that it has four or more peaks in its X-ray powder diffraction pattern selected from those at about 10.3, about 10.8, about 11.7, about 15.0, about 16.5, about 23.7, about 25.3, and about 26.6 degrees 2-theta. In some embodiments, Form A of compound 10 is characterized in that it has five or more peaks in its X-ray powder diffraction pattern selected from those at about 10.3, about 10.8, about 11.7, about 15.0, about 16.5, about 23.7, about 25.3, and about 26.6 degrees 2-theta. In some embodiments, Form A of compound 10 is characterized in that it has six or more peaks in its X-ray powder diffraction pattern selected from those at about 10.3, about 10.8, about 11.7, about 15.0, about 16.5, about 23.7, about 25.3, and about 26.6 degrees 2-theta. In some embodiments, Form A of compound 10 is characterized in that it has seven or more peaks in its X-ray powder diffraction pattern selected from those at about 10.3, about 10.8, about 11.7, about 15.0, about 16.5, about 23.7, about 25.3, and about 26.6 degrees 2-theta. In some embodiments, Form A of compound 10 is characterized in that it has eight peaks in its X-ray powder diffraction pattern selected from those at about 10.3, about 10.8, about 11.7, about 15.0, about 16.5, about 23.7, about 25.3, and about 26.6 degrees 2-theta.

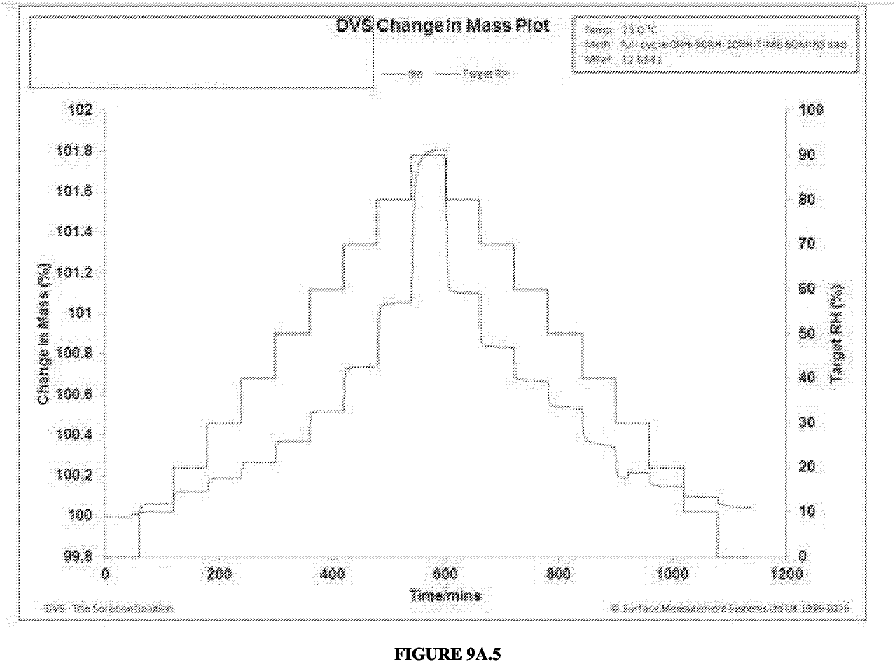

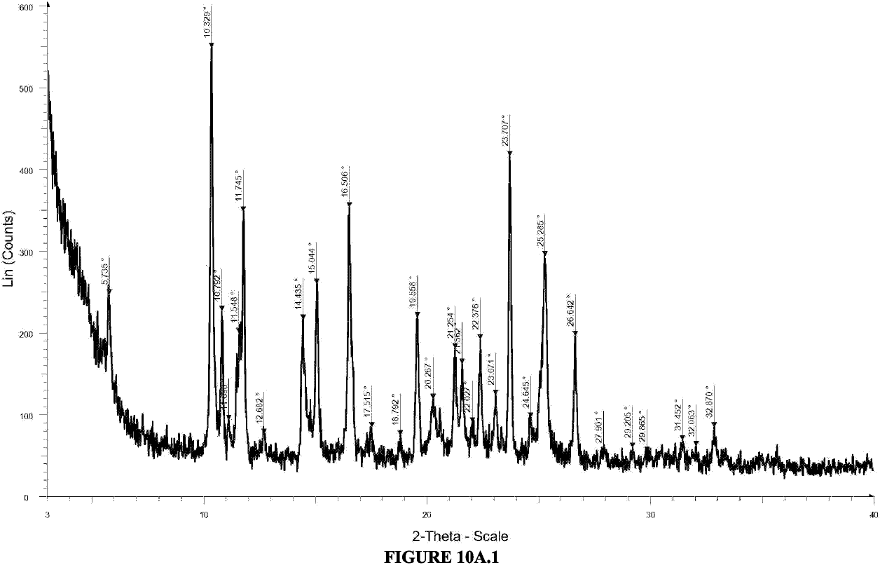

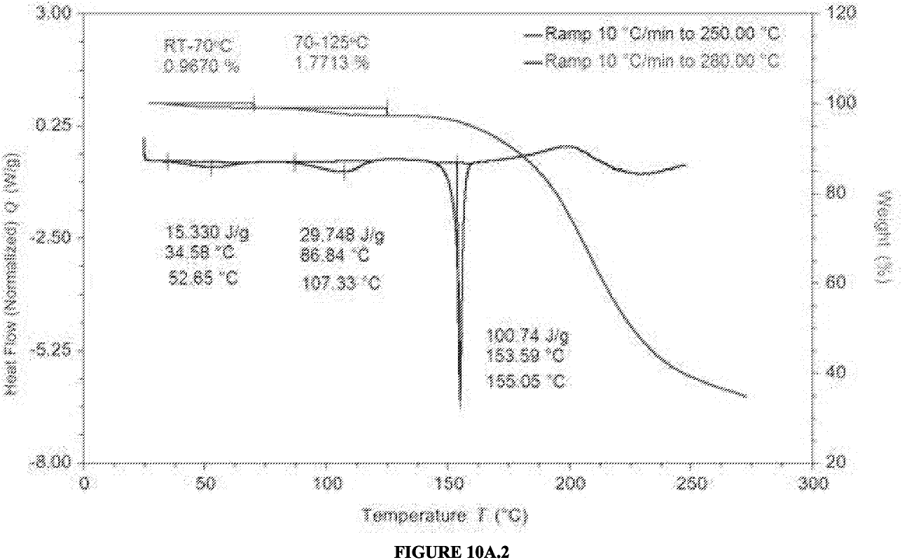

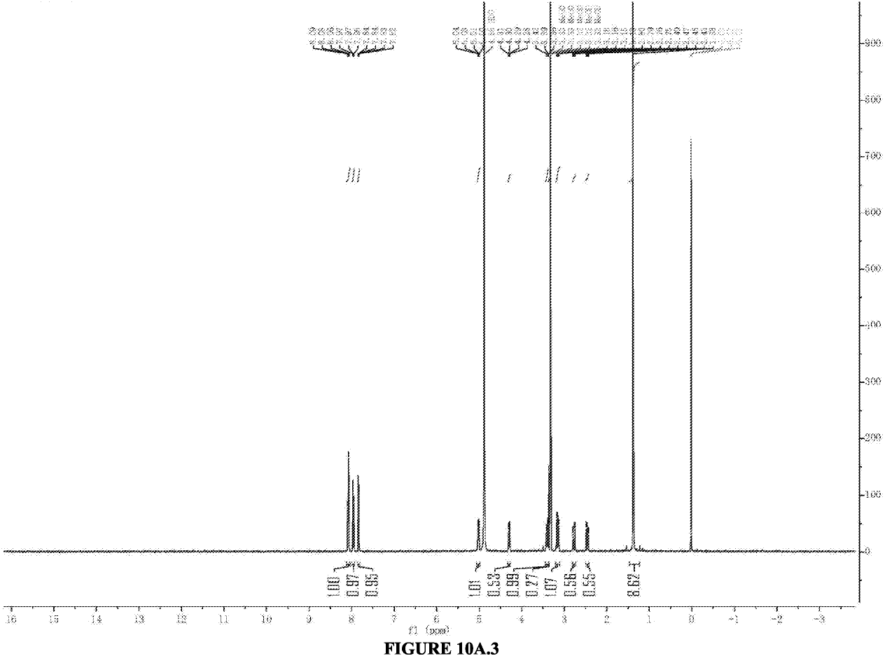

In some embodiments, Form A of compound 10 is characterized in that it has each of the spectral peaks in its X-ray powder diffraction pattern listed in Table 11 having a relative intensity greater than 10%, 20%, 30% or 40%. In certain embodiments, the X-ray powder diffraction pattern is substantially similar to the XRPD provided in FIG. 10A.1.

Methods for preparing Form A of compound 10 are described infra.

In some embodiments, the disclosure provides compound 10:

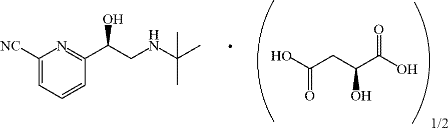

wherein said compound is crystalline. In some embodiments, the present disclosure provides compound 10, wherein said compound is substantially free of amorphous compound 10.

In some embodiments, the present disclosure provides compound 10, wherein said compound is substantially free of impurities.

In some embodiments, the present disclosure provides compound 10, wherein said compound has one or more peaks in its XRPD selected from those at about 10.3, about 10.8, about 11.7, about 15.0, about 16.5, about 23.7, about 25.3, and about 26.6 degrees 2-theta. In some such embodiments, the present disclosure provides compound 10, wherein said compound has at least two peaks in its XRPD selected from those at about 10.3, about 10.8, about 11.7, about 15.0, about 16.5, about 23.7, about 25.3, and about 26.6 degrees 2-theta. In some such embodiments, the present disclosure provides compound 10, wherein said compound is of Form A.

In some embodiments, the present disclosure provides compound 10, wherein said compound has an XRPD substantially similar to that depicted in FIG. 10A.1.

In some embodiments, the present disclosure provides a composition comprising compound 10 and a pharmaceutically acceptable carrier or excipient.

In some embodiments, the present disclosure provides a method of activating an adrenergic receptor in a patient comprising administering to said patient compound 10 or a composition thereof. In some embodiments, the adrenergic receptor is selected from β1-adrenergic receptor and β2-adrenergic receptor.

In some embodiments, the present disclosure provides a method of treating a β1-adrenergic receptor or β2-adrenergic receptor mediated disease or disorder in a patient, comprising administering to said patient compound 10 or composition thereof.

### Compound 11 (D-mandelate Salts of Compound 1)

According to one embodiment, the present disclosure provides a D-mandelate salt of compound 1, represented by compound 11:

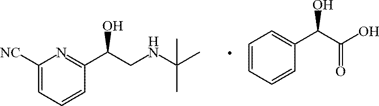

It will be appreciated by one of ordinary skill in the art that the D-mandelic acid and compound 1 are ionically bonded to form compound 11. It is contemplated that compound 11 can exist in a variety of physical forms. For example, compound 11 can be in solution, suspension, or in solid form. In certain embodiments, compound 11 is in solid form. When compound 11 is in solid form, said compound may be amorphous, crystalline, or a mixture thereof. Exemplary solid forms are described in more detail below.

In some embodiments, the present disclosure provides a form of compound 11 substantially free of impurities. As used herein, the term “substantially free of impurities” means that the compound contains no significant amount of extraneous matter. Such extraneous matter may include different forms of compound 11, residual solvents, or any other impurities that may result from the preparation of, and/or isolation of, compound 11. In certain embodiments, at least about 95% by weight of a form of compound 11 is present. In still other embodiments of the disclosure, at least about 99% by weight of a form of compound 11 is present.

According to one embodiment, a form of compound 11 is present in an amount of at least about 97, 97.5, 98.0, 98.5, 99, 99.5, 99.8 weight percent where the percentages are based on the total weight of the composition. According to another embodiment, a form of compound 11 contains no more than about 3.0 area percent HPLC of total organic impurities and, in certain embodiments, no more than about 1.5 area percent HPLC total organic impurities relative to the total area of the HPLC chromatogram. In other embodiments, a form of compound 11 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 11 is also meant to include all tautomeric forms of compound 11. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 11 can exist in a variety of solid forms. Exemplary such forms include polymorphs such as those described herein.

In certain embodiments, compound 11 is a crystalline solid. In other embodiments, compound 11 is a crystalline solid substantially free of amorphous compound 11. As used herein, the term “substantially free of amorphous compound 11” means that the compound contains no significant amount of amorphous compound 11. In certain embodiments, at least about 95% by weight of crystalline compound 11 is present. In still other embodiments of the disclosure, at least about 99% by weight of crystalline compound 11 is present.

It has been found that compound 11 can exist in at least one distinct polymorphic form. In certain embodiments, the present disclosure provides a polymorphic form of compound 11 referred to herein as Form A.

In some embodiments, compound 11 is amorphous. In some embodiments, compound 11 is amorphous, and is substantially free of crystalline compound 11.

### Form A of Compound 11

In some embodiments, Form A of compound 11 has at least 1, 2, 3, 4 or 5 spectral peak(s) selected from the peaks listed in Table 12 below.

TABLE 12

XRPD Peak Positions for Form A of Compound 11

| Position (°2θ) | Intensity % | Position (°2θ) | Intensity % |
|---|---|---|---|
| 6.5 | 69.6 | 23.7 | 2.0 |
| 7.4 | 8.9 | 26.3 | 22.3 |
| 9.0 | 7.1 | 27.0 | 2.9 |
| 13.1 | 13.9 | 27.4 | 17.0 |
| 14.0 | 34.9 | 28.3 | 6.8 |
| 14.8 | 3.0 | 31.1 | 2.4 |
| 15.2 | 4.6 | 31.9 | 2.3 |
| 16.1 | 3.9 | 32.9 | 2.0 |
| 17.2 | 2.9 | 34.3 | 8.1 |
| 19.8 | 8.9 | 37.2 | 1.9 |
| 20.0 | 5.4 | 39.7 | 3.4 |
| 22.8 | 100.0 | | |

In this and all subsequent tables, the position (°2θ) is within ± 0.2.

In some embodiments, Form A of compound 11 is characterized in that it has one or more peaks in its X-ray powder diffraction pattern selected from those at about 6.5, about 14.0, about 22.8, and about 26.3 degrees 2-theta. In some embodiments, Form A of compound 11 is characterized in that it has two or more peaks in its X-ray powder diffraction pattern selected from those at about 6.5, about 14.0, about 22.8, and about 26.3 degrees 2-theta. In some embodiments, Form A of compound 11 is characterized in that it has three or more peaks in its X-ray powder diffraction pattern selected from those at about 6.5, about 14.0, about 22.8, and about 26.3 degrees 2-theta. In some embodiments, Form A of compound 11 is characterized in that it has four peaks in its X-ray powder diffraction pattern selected from those at about 6.5, about 14.0, about 22.8, and about 26.3 degrees 2-theta.

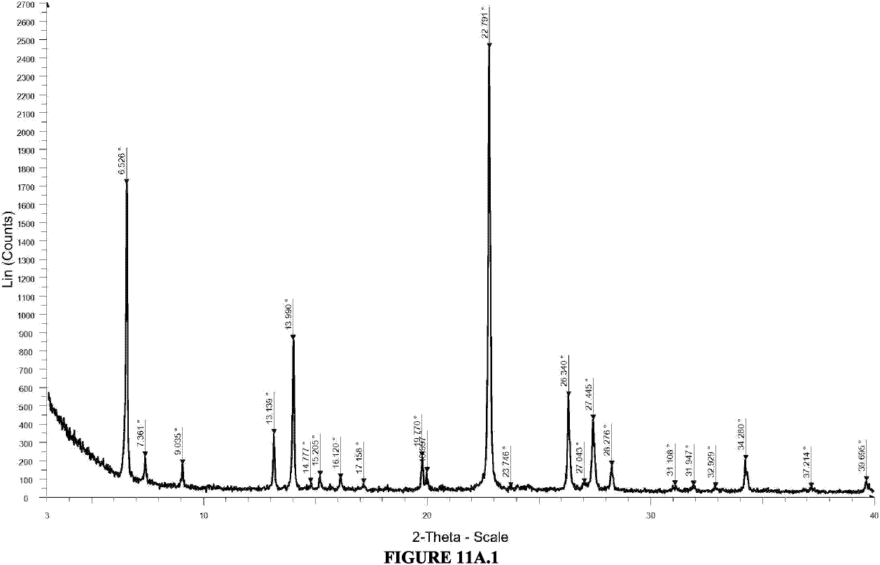

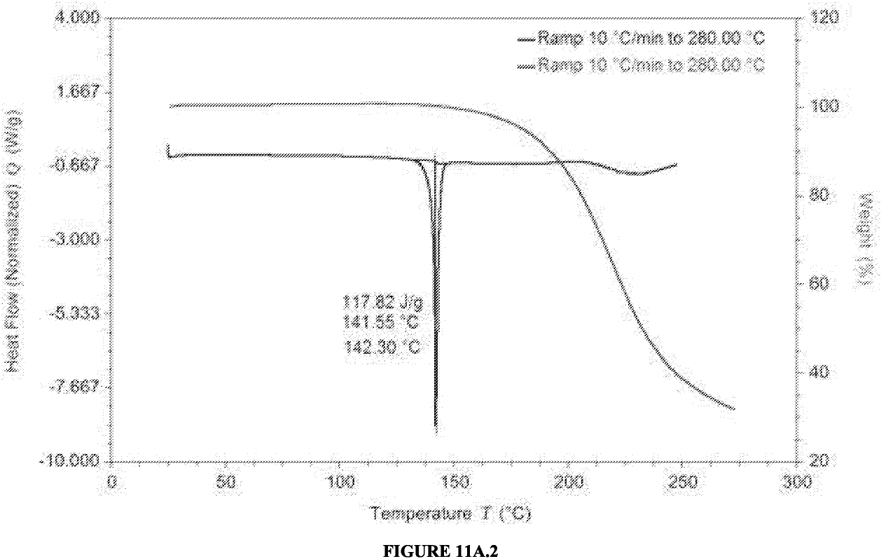

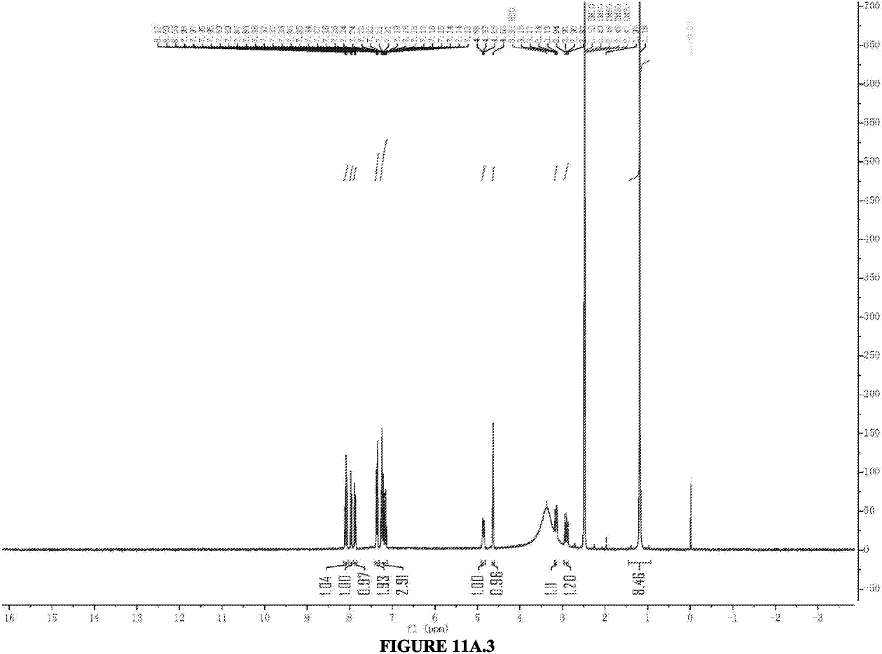

In some embodiments, Form A of compound 11 is characterized in that it has each of the spectral peaks in its X-ray powder diffraction pattern listed in Table 12 having a relative intensity greater than 10%, 20%, 30% or 40%. In certain embodiments, the X-ray powder diffraction pattern is substantially similar to the XRPD provided in FIG. 11A.1.

Methods for preparing Form A of compound 11 are described infra.

In some embodiments, the disclosure provides compound 11:

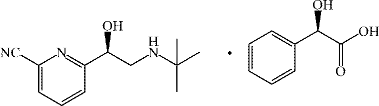

wherein said compound is crystalline. In some embodiments, the present disclosure provides compound 11, wherein said compound is substantially free of amorphous compound 11.

In some embodiments, the present disclosure provides compound 11, wherein said compound is substantially free of impurities.

In some embodiments, the present disclosure provides compound 11, wherein said compound has one or more peaks in its XRPD selected from those at about 6.5, about 14.0, about 22.8, and about 26.3 degrees 2-theta. In some such embodiments, the present disclosure provides compound 11, wherein said compound has at least two peaks in its XRPD selected from those at about 6.5, about 14.0, about 22.8, and about 26.3 degrees 2-theta. In some such embodiments, the present disclosure provides compound 11, wherein said compound is of Form A.

In some embodiments, the present disclosure provides compound 11, wherein said compound has an XRPD substantially similar to that depicted in FIG. 11A.1.

In some embodiments, the present disclosure provides a composition comprising compound 11 and a pharmaceutically acceptable carrier or excipient.

In some embodiments, the present disclosure provides a method of activating an adrenergic receptor in a patient comprising administering to said patient compound 11 or a composition thereof. In some embodiments, the adrenergic receptor is selected from β1-adrenergic receptor and β2-adrenergic receptor.

In some embodiments, the present disclosure provides a method of treating a β1-adrenergic receptor or β2-adrenergic receptor mediated disease or disorder in a patient, comprising administering to said patient compound 11 or composition thereof.

### Compound 12 (L-lactate Salts of Compound 1)

According to one embodiment, the present disclosure provides an L-lactate salt of compound 1, represented by compound 12:

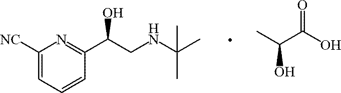

It will be appreciated by one of ordinary skill in the art that the L-lactic acid and compound 1 are ionically bonded to form compound 12. It is contemplated that compound 12 can exist in a variety of physical forms. For example, compound 12 can be in solution, suspension, or in solid form. In certain embodiments, compound 12 is in solid form. When compound 12 is in solid form, said compound may be amorphous, crystalline, or a mixture thereof. Exemplary solid forms are described in more detail below.

In some embodiments, the present disclosure provides a form of compound 12 substantially free of impurities. As used herein, the term “substantially free of impurities” means that the compound contains no significant amount of extraneous matter. Such extraneous matter may include different forms of compound 12, residual solvents, or any other impurities that may result from the preparation of, and/or isolation of, compound 12. In certain embodiments, at least about 95% by weight of a form of compound 12 is present. In still other embodiments of the disclosure, at least about 99% by weight of a form of compound 12 is present.

According to one embodiment, a form of compound 12 is present in an amount of at least about 97, 97.5, 98.0, 98.5, 99, 99.5, 99.8 weight percent where the percentages are based on the total weight of the composition. According to another embodiment, a form of compound 12 contains no more than about 3.0 area percent HPLC of total organic impurities and, in certain embodiments, no more than about 1.5 area percent HPLC total organic impurities relative to the total area of the HPLC chromatogram. In other embodiments, a form of compound 12 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 12 is also meant to include all tautomeric forms of compound 12. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 12 can exist in a variety of solid forms. Exemplary such forms include polymorphs such as those described herein.

In certain embodiments, compound 12 is a crystalline solid. In other embodiments, compound 12 is a crystalline solid substantially free of amorphous compound 12. As used herein, the term “substantially free of amorphous compound 12” means that the compound contains no significant amount of amorphous compound 12. In certain embodiments, at least about 95% by weight of crystalline compound 12 is present. In still other embodiments of the disclosure, at least about 99% by weight of crystalline compound 12 is present.

It has been found that compound 12 can exist in at least one distinct polymorphic form. In certain embodiments, the present disclosure provides a polymorphic form of compound 12 referred to herein as Form A.

In some embodiments, compound 12 is amorphous. In some embodiments, compound 12 is amorphous, and is substantially free of crystalline compound 12.

### Form A of Compound 12

In some embodiments, Form A of compound 12 has at least 1, 2, 3, 4 or 5 spectral peak(s) selected from the peaks listed in Table 13 below.

TABLE 13

XRPD Peak Positions for Form A of Compound 12

| Position (°2θ) | Intensity % | Position (°2θ) | Intensity % |
|---|---|---|---|
| 5.0 | 58.5 | 22.1 | 59.5 |
| 6.4 | 18.5 | 22.8 | 27.9 |
| 7.1 | 15.0 | 23.2 | 19.1 |
| 10.1 | 100.0 | 24.0 | 15.8 |
| 10.4 | 77.7 | 24.6 | 29.6 |
| 10.8 | 47.7 | 25.0 | 19.3 |
| 11.1 | 56.6 | 25.6 | 29.6 |
| 13.7 | 25.1 | 25.8 | 19.4 |
| 15.2 | 28.3 | 26.4 | 20.1 |
| 15.7 | 23.2 | 27.7 | 22.3 |
| 16.2 | 53.1 | 28.6 | 16.9 |
| 17.0 | 24.1 | 29.1 | 13.4 |
| 17.4 | 26.3 | 30.2 | 9.9 |
| 17.7 | 27.9 | 31.0 | 13.9 |
| 19.1 | 16.0 | 31.3 | 13.4 |
| 19.8 | 24.0 | 31.9 | 12.4 |
| 20.4 | 29.2 | 32.6 | 12.5 |
| 21.3 | 65.0 | 35.8 | 11.6 |

In this and all subsequent tables, the position (°2θ) is within ± 0.2.

In some embodiments, Form A of compound 12 is characterized in that it has one or more peaks in its X-ray powder diffraction pattern selected from those at about 5.0, about 10.1, about 10.4, about 10.8, about 11.1, about 16.2, about 21.3, and about 22.1 degrees 2-theta. In some embodiments, Form A of compound 12 is characterized in that it has two or more peaks in its X-ray powder diffraction pattern selected from those at about 5.0, about 10.1, about 10.4, about 10.8, about 11.1, about 16.2, about 21.3, and about 22.1 degrees 2-theta. In some embodiments, Form A of compound 12 is characterized in that it has three or more peaks in its X-ray powder diffraction pattern selected from those at about 5.0, about 10.1, about 10.4, about 10.8, about 11.1, about 16.2, about 21.3, and about 22.1 degrees 2-theta. In some embodiments, Form A of compound 12 is characterized in that it has four or more peaks in its X-ray powder diffraction pattern selected from those at about 5.0, about 10.1, about 10.4, about 10.8, about 11.1, about 16.2, about 21.3, and about 22.1 degrees 2-theta. In some embodiments, Form A of compound 12 is characterized in that it has five or more peaks in its X-ray powder diffraction pattern selected from those at about 5.0, about 10.1, about 10.4, about 10.8, about 11.1, about 16.2, about 21.3, and about 22.1 degrees 2-theta. In some embodiments, Form A of compound 12 is characterized in that it has six or more peaks in its X-ray powder diffraction pattern selected from those at about 5.0, about 10.1, about 10.4, about 10.8, about 11.1, about 16.2, about 21.3, and about 22.1 degrees 2-theta. In some embodiments, Form A of compound 12 is characterized in that it has seven or more peaks in its X-ray powder diffraction pattern selected from those at about 5.0, about 10.1, about 10.4, about 10.8, about 11.1, about 16.2, about 21.3, and about 22.1 degrees 2-theta. In some embodiments, Form A of compound 12 is characterized in that it has eight peaks in its X-ray powder diffraction pattern selected from those at about 5.0, about 10.1, about 10.4, about 10.8, about 11.1, about 16.2, about 21.3, and about 22.1 degrees 2-theta.

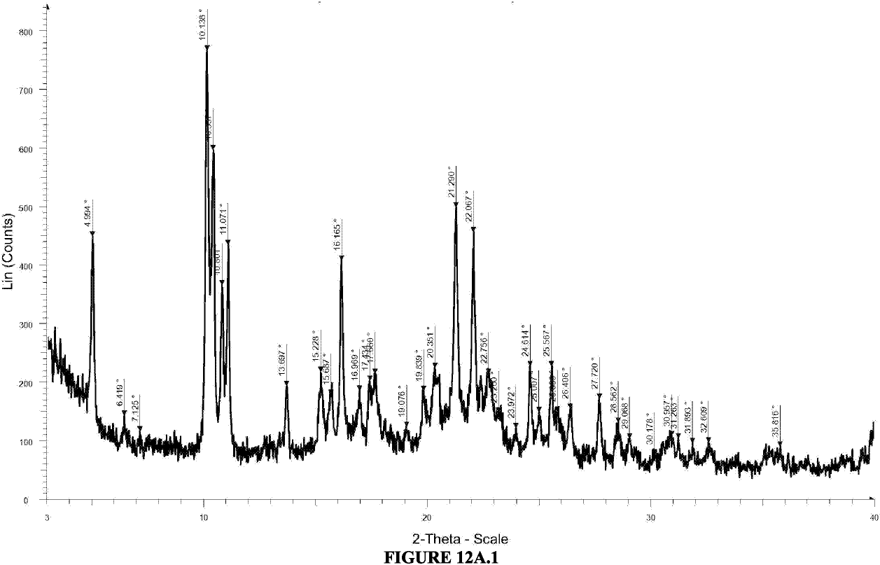

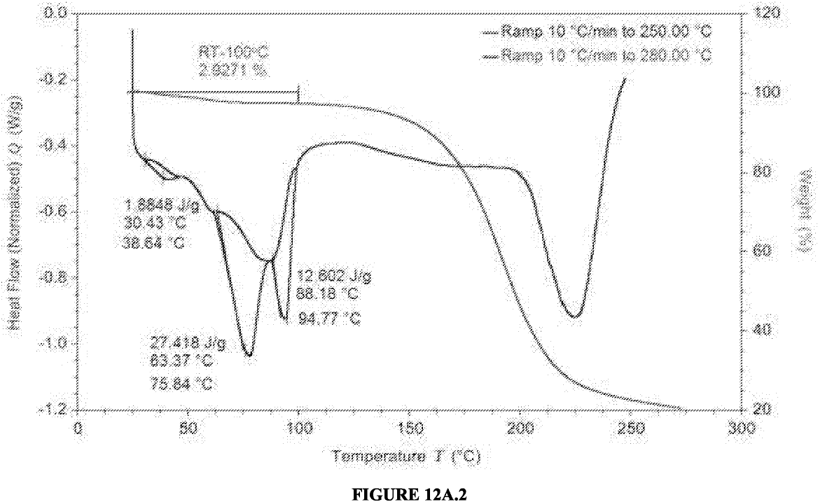

In some embodiments, Form A of compound 12 is characterized in that it has each of the spectral peaks in its X-ray powder diffraction pattern listed in Table 13 having a relative intensity greater than 10%, 20%, 30% or 40%. In certain embodiments, the X-ray powder diffraction pattern is substantially similar to the XRPD provided in FIG. 12A.1.

Methods for preparing Form A of compound 12 are described infra.

In some embodiments, the disclosure provides compound 12:

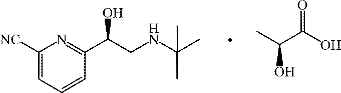

wherein said compound is crystalline. In some embodiments, the present disclosure provides compound 12, wherein said compound is substantially free of amorphous compound 12.

In some embodiments, the present disclosure provides compound 12, wherein said compound is substantially free of impurities.

In some embodiments, the present disclosure provides compound 12, wherein said compound has one or more peaks in its XRPD selected from those at about 5.0, about 10.1, about 10.4, about 10.8, about 11.1, about 16.2, about 21.3, and about 22.1 degrees 2-theta. In some such embodiments, the present disclosure provides compound 12, wherein said compound has at least two peaks in its XRPD selected from those at about 5.0, about 10.1, about 10.4, about 10.8, about 11.1, about 16.2, about 21.3, and about 22.1 degrees 2-theta. In some such embodiments, the present disclosure provides compound 12, wherein said compound is of Form A.

In some embodiments, the present disclosure provides compound 12, wherein said compound has an XRPD substantially similar to that depicted in FIG. 12A.1.

In some embodiments, the present disclosure provides a composition comprising compound 12 and a pharmaceutically acceptable carrier or excipient.

In some embodiments, the present disclosure provides a method of activating an adrenergic receptor in a patient comprising administering to said patient compound 12 or a composition thereof. In some embodiments, the adrenergic receptor is selected from β1-adrenergic receptor and β2-adrenergic receptor.

In some embodiments, the present disclosure provides a method of treating a β1-adrenergic receptor or β2-adrenergic receptor mediated disease or disorder in a patient, comprising administering to said patient compound 12 or composition thereof.

### Compound 13 (D-camphorate Salts of Compound 1)

According to one embodiment, the present disclosure provides a D-camphorate salt of compound 1, represented by compound 13:

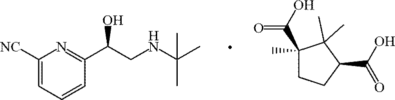

It will be appreciated by one of ordinary skill in the art that the D-camphoric acid and compound 1 are ionically bonded to form compound 13. It is contemplated that compound 13 can exist in a variety of physical forms. For example, compound 13 can be in solution, suspension, or in solid form. In certain embodiments, compound 13 is in solid form. When compound 13 is in solid form, said compound may be amorphous, crystalline, or a mixture thereof. Exemplary solid forms are described in more detail below.

In some embodiments, the present disclosure provides a form of compound 13 substantially free of impurities. As used herein, the term “substantially free of impurities” means that the compound contains no significant amount of extraneous matter. Such extraneous matter may include different forms of compound 13, residual solvents, or any other impurities that may result from the preparation of, and/or isolation of, compound 13. In certain embodiments, at least about 95% by weight of a form of compound 13 is present. In still other embodiments of the disclosure, at least about 99% by weight of a form of compound 13 is present.

According to one embodiment, a form of compound 13 is present in an amount of at least about 97, 97.5, 98.0, 98.5, 99, 99.5, 99.8 weight percent where the percentages are based on the total weight of the composition. According to another embodiment, a form of compound 13 contains no more than about 3.0 area percent HPLC of total organic impurities and, in certain embodiments, no more than about 1.5 area percent HPLC total organic impurities relative to the total area of the HPLC chromatogram. In other embodiments, a form of compound 13 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 13 is also meant to include all tautomeric forms of compound 13. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 13 can exist in a variety of solid forms. Exemplary such forms include polymorphs such as those described herein.

In certain embodiments, compound 13 is a crystalline solid. In other embodiments, compound 13 is a crystalline solid substantially free of amorphous compound 13. As used herein, the term “substantially free of amorphous compound 13” means that the compound contains no significant amount of amorphous compound 13. In certain embodiments, at least about 95% by weight of crystalline compound 13 is present. In still other embodiments of the disclosure, at least about 99% by weight of crystalline compound 13 is present.

It has been found that compound 13 can exist in at least one distinct polymorphic form. In certain embodiments, the present disclosure provides a polymorphic form of compound 13 referred to herein as Form A.

In some embodiments, compound 13 is amorphous. In some embodiments, compound 13 is amorphous, and is substantially free of crystalline compound 13.

### Form A of Compound 13

In some embodiments, Form A of compound 13 has at least 1, 2, 3, 4 or 5 spectral peak(s) selected from the peaks listed in Table 14 below.

TABLE 14

XRPD Peak Positions for Form A of Compound 13

| Position (°2θ) | Intensity % | Position (°2θ) | Intensity % |
|---|---|---|---|
| 4.5 | 10.9 | 22.2 | 3.2 |
| 7.9 | 100.0 | 23.2 | 4.0 |
| 8.6 | 10.3 | 23.6 | 3.4 |
| 9.5 | 35.3 | 24.9 | 3.6 |
| 10.1 | 7.6 | 25.6 | 3.7 |
| 10.9 | 3.6 | 26.1 | 6.1 |
| 11.7 | 23.7 | 26.5 | 4.7 |
| 12.9 | 19.4 | 27.9 | 2.8 |
| 13.3 | 2.5 | 28.6 | 2.8 |
| 14.7 | 20.4 | 29.1 | 2.6 |
| 15.7 | 3.6 | 29.4 | 3.9 |
| 16.2 | 10.0 | 29.7 | 5.9 |
| 16.8 | 13.1 | 31.0 | 5.3 |
| 17.2 | 21.4 | 32.0 | 1.9 |
| 17.8 | 4.9 | 32.9 | 2.3 |
| 18.1 | 11.3 | 33.1 | 3.4 |
| 18.5 | 20.5 | 34.8 | 1.9 |
| 18.9 | 21.6 | 36.5 | 2.0 |
| 19.1 | 13.4 | 37.4 | 2.8 |
| 20.3 | 3.8 | 37.7 | 2.2 |
| 20.6 | 3.9 | 38.8 | 2.2 |
| 21.1 | 7.5 | 39.6 | 2.1 |
| 21.8 | 7.2 | | |

In this and all subsequent tables, the position (°2θ) is within ± 0.2.

In some embodiments, Form A of compound 13 is characterized in that it has one or more peaks in its X-ray powder diffraction pattern selected from those at about 7.9, about 9.5, about 11.7, about 14.737, about 17.2, about 18.5, and about 18.9 degrees 2-theta. In some embodiments, Form A of compound 13 is characterized in that it has two or more peaks in its X-ray powder diffraction pattern selected from those at about 7.9, about 9.5, about 11.7, about 14.737, about 17.2, about 18.5, and about 18.9 degrees 2-theta. In some embodiments, Form A of compound 13 is characterized in that it has three or more peaks in its X-ray powder diffraction pattern selected from those at about 7.9, about 9.5, about 11.7, about 14.737, about 17.2, about 18.5, and about 18.9 degrees 2-theta. In some embodiments, Form A of compound 13 is characterized in that it has four or more peaks in its X-ray powder diffraction pattern selected from those at about 7.9, about 9.5, about 11.7, about 14.737, about 17.2, about 18.5, and about 18.9 degrees 2-theta. In some embodiments, Form A of compound 13 is characterized in that it has five or more peaks in its X-ray powder diffraction pattern selected from those at about 7.9, about 9.5, about 11.7, about 14.737, about 17.2, about 18.5, and about 18.9 degrees 2-theta. In some embodiments, Form A of compound 13 is characterized in that it has six or more peaks in its X-ray powder diffraction pattern selected from those at about 7.9, about 9.5, about 11.7, about 14.737, about 17.2, about 18.5, and about 18.9 degrees 2-theta. In some embodiments, Form A of compound 13 is characterized in that it has seven peaks in its X-ray powder diffraction pattern selected from those at about 7.9, about 9.5, about 11.7, about 14.737, about 17.2, about 18.5, and about 18.9 degrees 2-theta.

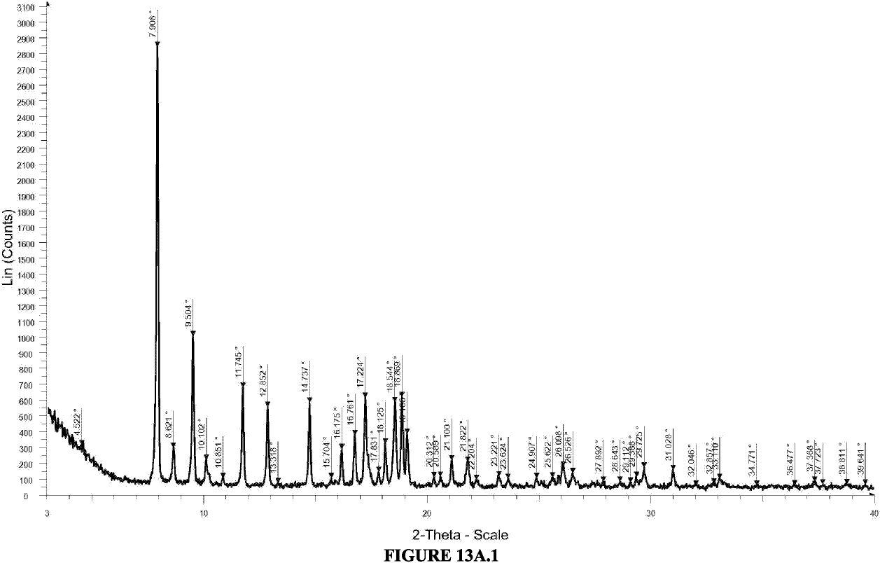

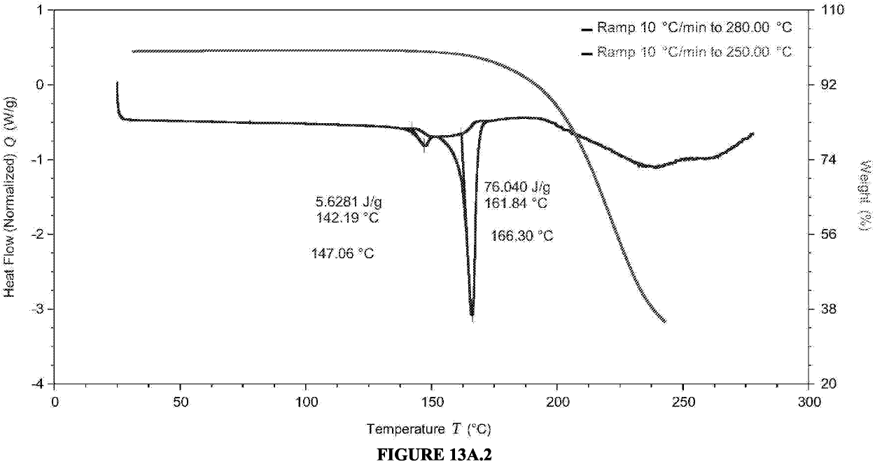

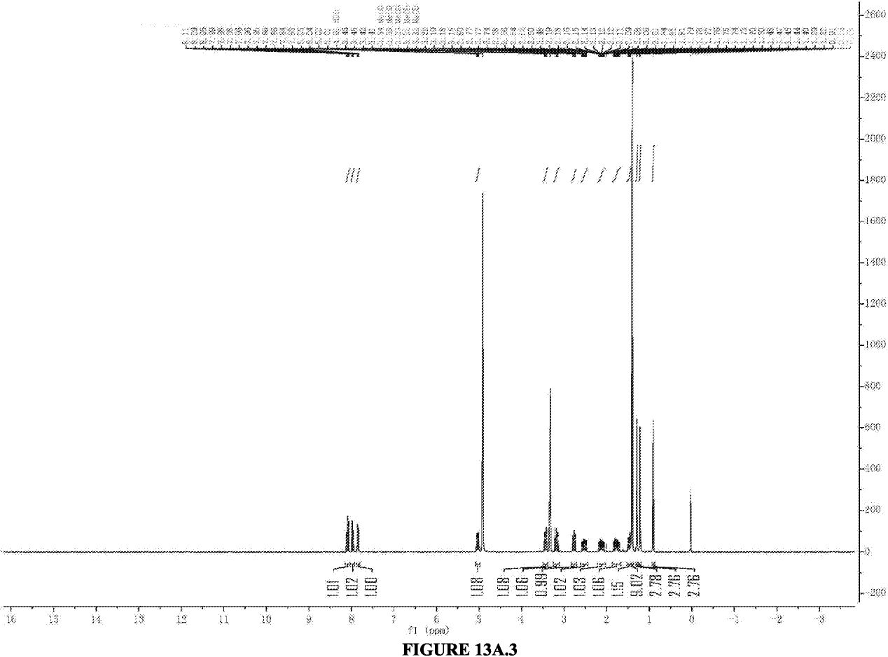

In some embodiments, Form A of compound 13 is characterized in that it has each of the spectral peaks in its X-ray powder diffraction pattern listed in Table 14 having a relative intensity greater than 10%, 20%, 30% or 40%. In certain embodiments, the X-ray powder diffraction pattern is substantially similar to the XRPD provided in FIG. 13A.1.

Methods for preparing Form A of compound 13 are described infra.

In some embodiments, the disclosure provides compound 13:

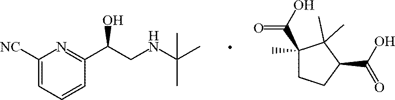

wherein said compound is crystalline. In some embodiments, the present disclosure provides compound 13, wherein said compound is substantially free of amorphous compound 13.

In some embodiments, the present disclosure provides compound 13, wherein said compound is substantially free of impurities.

In some embodiments, the present disclosure provides compound 13, wherein said compound has one or more peaks in its XRPD selected from those at about 7.9, about 9.5, about 11.7, about 14.737, about 17.2, about 18.5, and about 18.9 degrees 2-theta. In some such embodiments, the present disclosure provides compound 13, wherein said compound has at least two peaks in its XRPD selected from those at about 7.9, about 9.5, about 11.7, about 14.737, about 17.2, about 18.5, and about 18.9 degrees 2-theta. In some such embodiments, the present disclosure provides compound 13, wherein said compound is of Form A.

In some embodiments, the present disclosure provides compound 13, wherein said compound has an XRPD substantially similar to that depicted in FIG. 13A.1.

In some embodiments, the present disclosure provides a composition comprising compound 13 and a pharmaceutically acceptable carrier or excipient.

In some embodiments, the present disclosure provides a method of activating an adrenergic receptor in a patient comprising administering to said patient compound 13 or a composition thereof. In some embodiments, the adrenergic receptor is selected from β1-adrenergic receptor and β2-adrenergic receptor.

In some embodiments, the present disclosure provides a method of treating a β1-adrenergic receptor or β2-adrenergic receptor mediated disease or disorder in a patient, comprising administering to said patient compound 13 or composition thereof.

### Compound 14 (Dibenzoyl-D-tartrate Salts of Compound 1)

According to one embodiment, the present disclosure provides an dibenzoyl-D-tartrate salt of compound 1, represented by compound 14:

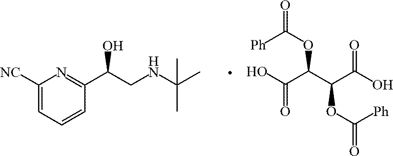

It will be appreciated by one of ordinary skill in the art that the dibenzoyl-D-tartaric acid and compound 1 are ionically bonded to form compound 14. It is contemplated that compound 14 can exist in a variety of physical forms. For example, compound 14 can be in solution, suspension, or in solid form. In certain embodiments, compound 14 is in solid form. When compound 14 is in solid form, said compound may be amorphous, crystalline, or a mixture thereof. Exemplary solid forms are described in more detail below.

In some embodiments, the present disclosure provides a form of compound 14 substantially free of impurities. As used herein, the term “substantially free of impurities” means that the compound contains no significant amount of extraneous matter. Such extraneous matter may include different forms of compound 14, residual solvents, or any other impurities that may result from the preparation of, and/or isolation of, compound 14. In certain embodiments, at least about 95% by weight of a form of compound 14 is present. In still other embodiments of the disclosure, at least about 99% by weight of a form of compound 14 is present.

According to one embodiment, a form of compound 14 is present in an amount of at least about 97, 97.5, 98.0, 98.5, 99, 99.5, 99.8 weight percent where the percentages are based on the total weight of the composition. According to another embodiment, a form of compound 14 contains no more than about 3.0 area percent HPLC of total organic impurities and, in certain embodiments, no more than about 1.5 area percent HPLC total organic impurities relative to the total area of the HPLC chromatogram. In other embodiments, a form of compound 14 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of compound 14 is also meant to include all tautomeric forms of compound 14. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

It has been found that compound 14 can exist in a variety of solid forms. Exemplary such forms include polymorphs such as those described herein.

In certain embodiments, compound 14 is a crystalline solid. In other embodiments, compound 14 is a crystalline solid substantially free of amorphous compound 14. As used herein, the term “substantially free of amorphous compound 14” means that the compound contains no significant amount of amorphous compound 14. In certain embodiments, at least about 95% by weight of crystalline compound 14 is present. In still other embodiments of the disclosure, at least about 99% by weight of crystalline compound 14 is present.

It has been found that compound 14 can exist in at least one distinct polymorphic form. In certain embodiments, the present disclosure provides a polymorphic form of compound 14 referred to herein as Form A.

In some embodiments, compound 14 is amorphous. In some embodiments, compound 14 is amorphous, and is substantially free of crystalline compound 14.

### Form A of Compound 14

In some embodiments, Form A of compound 14 has at least 1, 2, 3, 4 or 5 spectral peak(s) selected from the peaks listed in Table 15 below.

TABLE 15

XRPD Peak Positions for Form A of Compound 14

| Position (°2θ) | Intensity % | Position (°2θ) | Intensity % |
|---|---|---|---|
| 4.9 | 8.6 | 19.6 | 5.6 |
| 6.3 | 100.0 | 20.3 | 2.9 |
| 6.8 | 9.6 | 20.7 | 5.6 |
| 8.8 | 36.1 | 21.1 | 2.8 |
| 11.2 | 7.7 | 22.3 | 4.6 |
| 12.2 | 26.9 | 22.6 | 5.7 |
| 12.6 | 34.7 | 23.0 | 7.5 |
| 12.8 | 25.3 | 23.4 | 7.9 |
| 14.0 | 18.0 | 23.8 | 8.8 |
| 14.2 | 12.8 | 24.3 | 10.5 |
| 14.8 | 4.3 | 24.6 | 4.4 |
| 15.3 | 4.4 | 25.1 | 6.9 |
| 15.6 | 9.0 | 25.3 | 5.4 |
| 16.2 | 4.9 | 25.9 | 4.3 |
| 16.5 | 7.3 | 26.2 | 4.7 |
| 16.9 | 18.1 | 26.5 | 5.0 |
| 17.2 | 13.8 | 27.2 | 5.1 |
| 17.6 | 7.7 | 27.8 | 3.2 |
| 18.2 | 7.7 | 28.2 | 2.5 |
| 18.9 | 13.7 | 32.5 | 2.4 |
| 19.3 | 11.7 | | |

In this and all subsequent tables, the position (°2θ) is within ± 0.2.

In some embodiments, Form A of compound 14 is characterized in that it has one or more peaks in its X-ray powder diffraction pattern selected from those at about 6.3, about 8.8, about 12.2, about 12.6, and about 12.8 degrees 2-theta. In some embodiments, Form A of compound 14 is characterized in that it has two or more peaks in its X-ray powder diffraction pattern selected from those at about 6.3, about 8.8, about 12.2, about 12.6, and about 12.8 degrees 2-theta. In some embodiments, Form A of compound 14 is characterized in that it has three or more peaks in its X-ray powder diffraction pattern selected from those at about 6.3, about 8.8, about 12.2, about 12.6, and about 12.8 degrees 2-theta. In some embodiments, Form A of compound 14 is characterized in that it has four or more peaks in its X-ray powder diffraction pattern selected from those at about 6.3, about 8.8, about 12.2, about 12.6, and about 12.8 degrees 2-theta. In some embodiments, Form A of compound 14 is characterized in that it has five peaks in its X-ray powder diffraction pattern selected from those at about 6.3, about 8.8, about 12.2, about 12.6, and about 12.8 degrees 2-theta.

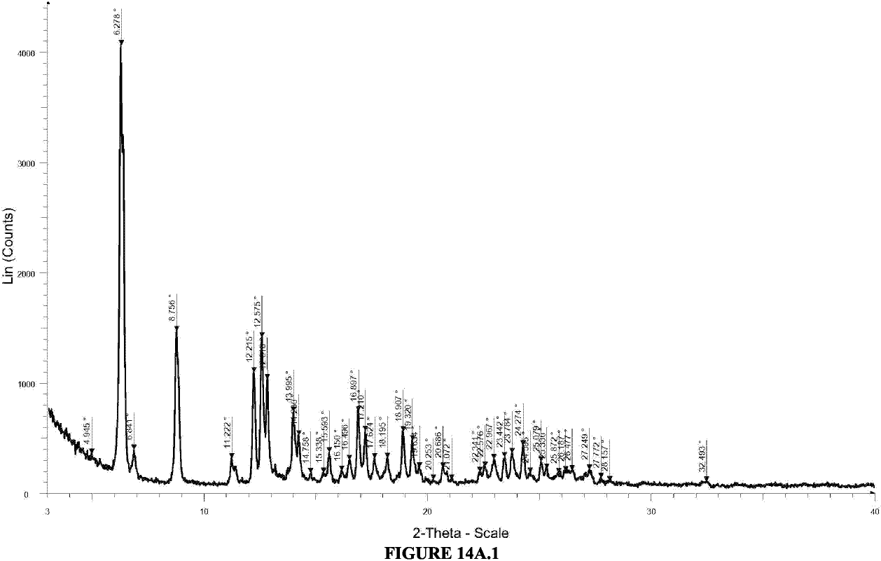

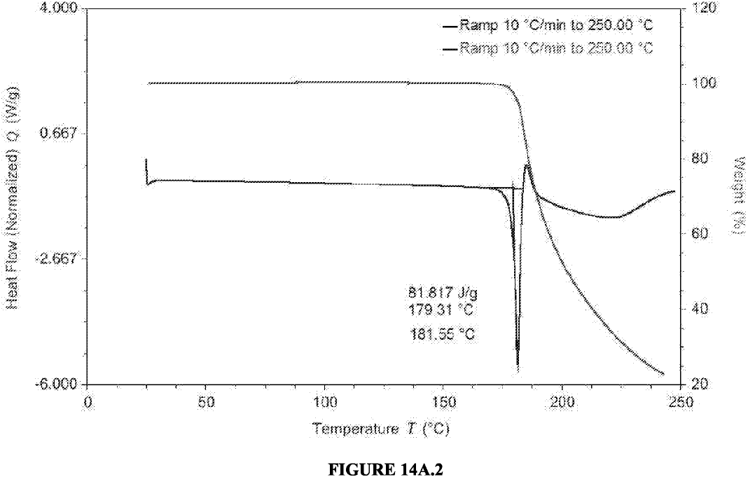

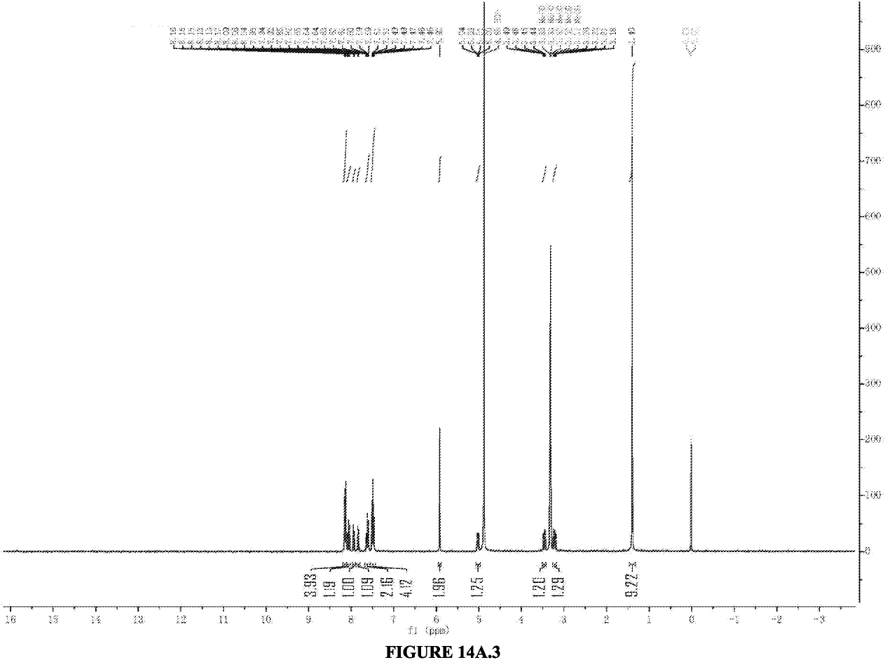

In some embodiments, Form A of compound 14 is characterized in that it has each of the spectral peaks in its X-ray powder diffraction pattern listed in Table 15 having a relative intensity greater than 10%, 20%, 30% or 40%. In certain embodiments, the X-ray powder diffraction pattern is substantially similar to the XRPD provided in FIG. 14A.1.

Methods for preparing Form A of compound 14 are described infra.

In some embodiments, the disclosure provides compound 14:

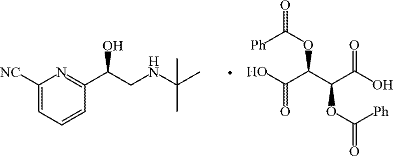

wherein said compound is crystalline. In some embodiments, the present disclosure provides compound 14, wherein said compound is substantially free of amorphous compound 14.

In some embodiments, the present disclosure provides compound 14, wherein said compound is substantially free of impurities.

In some embodiments, the present disclosure provides compound 14, wherein said compound has one or more peaks in its XRPD selected from those at about 6.3, about 8.8, about 12.2, about 12.6, and about 12.8 degrees 2-theta. In some such embodiments, the present disclosure provides compound 14, wherein said compound has at least two peaks in its XRPD selected from those at about 6.3, about 8.8, about 12.2, about 12.6, and about 12.8 degrees 2-theta. In some such embodiments, the present disclosure provides compound 14, wherein said compound is of Form A.

In some embodiments, the present disclosure provides compound 14, wherein said compound has an XRPD substantially similar to that depicted in FIG. 14A.1.

In some embodiments, the present disclosure provides a composition comprising compound 14 and a pharmaceutically acceptable carrier or excipient.

In some embodiments, the present disclosure provides a method of activating an adrenergic receptor in a patient comprising administering to said patient compound 14 or a composition thereof. In some embodiments, the adrenergic receptor is selected from β1-adrenergic receptor and β2-adrenergic receptor.

In some embodiments, the present disclosure provides a method of treating a β1-adrenergic receptor or β2-adrenergic receptor mediated disease or disorder in a patient, comprising administering to said patient compound 14 or composition thereof.

### General Methods of Providing a Salt Compound

Compound 1 can be prepared according to the general Scheme provided below:

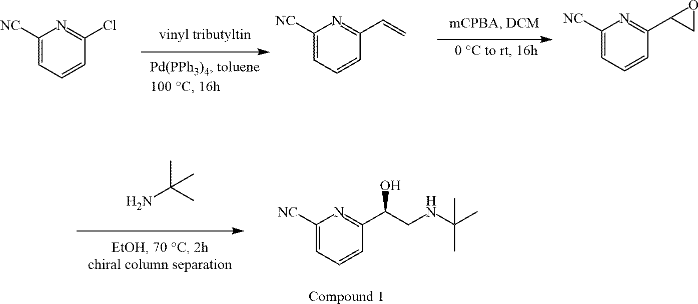

Salt compounds of general formula A, which formula encompasses, inter alia, salt compounds 2 through 12, and/or particular forms thereof, are prepared from compound 1, according to the general Scheme below.

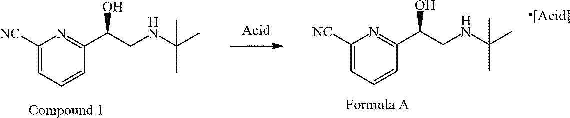

For instance, each of compounds 2 through 14, and forms thereof, are prepared from compound 1 by combining compound 1 with an appropriate acid to form a salt of that acid. Thus, another aspect of the present disclosure provides a method for preparing compounds 2 through 14, and forms thereof.

As described generally above, in some embodiments, the present disclosure provides a method for preparing a salt compound of the general formula A:

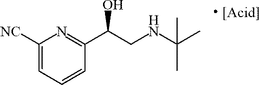

comprising steps of:
combining compound 1:

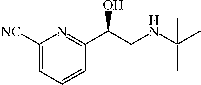

with a suitable acid and optionally a suitable solvent under conditions suitable for forming a salt of formula A.

In some embodiments, a suitable acid is hydrochloric acid. In some embodiments, the present disclosure provides a method of making a hydrochloride salt of compound 1. In certain embodiments, the hydrochloride salt of compound 1 is compound 2. In certain embodiments, the hydrochloride salt of compound 1 is Form A of compound 2. In certain embodiments, the hydrochloride salt of compound 1 is Form B of compound 2. In certain embodiments, the hydrochloride salt of compound 1 is Form C of compound 2.

In some embodiments, a suitable acid is sulfuric acid. In some embodiments, the present disclosure provides a method of making a sulfate salt of compound 1. In certain embodiments, the sulfate salt of compound 1 is compound 3. In certain embodiments, the sulfate salt of compound 1 is Form A of compound 3.

In some embodiments, a suitable acid is hydrobromic acid. In some embodiments, the present disclosure provides a method of making a hydrobromide salt of compound 1. In certain embodiments, the hydrobromide salt of compound 1 is compound 4. In certain embodiments, the hydrobromide salt of compound 1 is Form A of compound 4.

In some embodiments, a suitable acid is para-toluenesulfonic acid. In some embodiments, the present disclosure provides a method of making a tosylate salt of compound 1. In certain embodiments, the tosylate salt of compound 1 is compound 5. In certain embodiments, the tosylate salt of compound 1 is Form A of compound 5.

In some embodiments, a suitable acid is maleic acid. In some embodiments, the present disclosure provides a method of making a maleate salt of compound 1. In certain embodiments, the maleate salt of compound 1 is compound 6. In certain embodiments, the maleate salt of compound 1 is Form A of compound 6.

In some embodiments, a suitable acid is fumaric acid. In some embodiments, the present disclosure provides a method of making a fumarate salt of compound 1. In certain embodiments, the fumarate salt of compound 1 is compound 7. In certain embodiments, the fumarate salt of compound 1 is Form A of compound 7.

In some embodiments, a suitable acid is glycolic acid. In some embodiments, the present disclosure provides a method of making a glycolate salt of compound 1. In certain embodiments, the glycolate salt of compound 1 is compound 8. In certain embodiments, the glycolate salt of compound 1 is Form A of compound 8.

In some embodiments, a suitable acid is L-tartaric acid. In some embodiments, the present disclosure provides a method of making a L-tartrate salt of compound 1. In certain embodiments, the L-tartrate salt of compound 1 is compound 9. In certain embodiments, the L-tartrate salt of compound 1 is Form A of compound 9.

In some embodiments, a suitable acid is L-malic acid. In some embodiments, the present disclosure provides a method of making an L-malate salt of compound 1. In certain embodiments, the L-malate salt of compound 1 is compound 10. In certain embodiments, the L-malate salt of compound 1 is Form A of compound 10.

In some embodiments, a suitable acid is D-mandelic acid. In some embodiments, the present disclosure provides a method of making a D-mandelate salt of compound 1. In certain embodiments, the D-mandelate salt of compound 1 is compound 11. In certain embodiments, the D-mandelate salt of compound 1 is Form A of compound 11.

In some embodiments, a suitable acid is L-lactic acid. In some embodiments, the present disclosure provides a method of making an L-lactate salt of compound 1. In certain embodiments, the L-lactate salt of compound 1 is compound 12. In certain embodiments, the L-lactate salt of compound 1 is Form A of compound 12.

In some embodiments, a suitable acid is D-camphoric acid. In some embodiments, the present disclosure provides a method of making a D-camphorate salt of compound 1. In certain embodiments, the D-camphorate salt of compound 1 is compound 13. In certain embodiments, the D-camphorate salt of compound 1 is Form A of compound 13. In certain embodiments, the D-camphorate salt of compound 1 is Form B of compound 13.

In some embodiments, a suitable acid is dibenzoyl-D-tartaric acid. In some embodiments, the present disclosure provides a method of making a dibenzoyl-D-tartrate salt of compound 1. In certain embodiments, the dibenzoyl-D-tartrate salt of compound 1 is compound 14. In certain embodiments, the dibenzoyl-D-tartrate salt of compound 1 is Form A of compound 14.

A suitable solvent may be any solvent system (e.g., one solvent or a mixture of solvents) in which compound 1 and/or an acid are soluble or are at least partially soluble.

Examples of suitable solvents useful in the presently disclosed methods include, but are not limited to protic solvents, aprotic solvents, polar aprotic solvent, or mixtures thereof. In certain embodiments, suitable solvents include an ether, an ester, an alcohol, a ketone, or a mixture thereof. In some embodiments, the solvent is one or more organic alcohols. In some embodiments, the solvent is chlorinated. In some embodiments, the solvent is an aromatic solvent.

In certain embodiments, a suitable solvent is methanol, ethanol, isopropanol, or acetone wherein said solvent is anhydrous or in combination with water or heptane. In some embodiments, suitable solvents include tetrahydrofuran, dimethyl formamide, dimethyl sulfoxide, glyme, diglyme, methyl t-butyl ether, t-butanol, n-butanol, and acetonitrile. In some embodiments, a suitable solvent is ethanol. In some embodiments, a suitable solvent is anhydrous ethanol. In some embodiments, the suitable solvent is MTBE.

In some embodiments, a suitable solvent is ethyl acetate. In some embodiments, a suitable solvent is methanol. In some embodiments, a suitable solvent is methylene chloride. In some embodiments, a suitable solvent is acetonitrile. In some embodiments, a suitable solvent is isopropanol. In certain embodiments, a suitable solvent is methyl acetate, isopropyl acetate, acetone, or tetrahydrofuran. In certain embodiments, a suitable solvent is diethyl ether. In certain embodiments, a suitable solvent is water. In certain embodiments, a suitable solvent is methyl ethyl ketone. In certain embodiments, a suitable solvent is toluene.

In some embodiments, the present disclosure provides a method for preparing a salt compound of the general formula A, comprising one or more steps of removing a solvent and adding a solvent. In some embodiments, an added solvent is the same as the solvent removed. In some embodiments, an added solvent is different from the solvent removed. Means of solvent removal are known in the synthetic and chemical arts and include, but are not limited to, any of those described herein and in the Examples.

In some embodiments, a method for preparing a salt compound of the general formula A comprises one or more steps of heating or cooling a preparation.

In some embodiments, a method for preparing a salt compound of the general formula A comprises one or more steps of agitating or stirring a preparation.

In some embodiments, a method for preparing a salt compound of the general formula A comprises slow evaporation of the solvent. In some embodiments, a method for preparing a salt compound of the general formula A comprises slow evaporation of the solvent through exposure to ambient atmosphere at room temperature. In some embodiments, a method for preparing a salt compound of the general formula A comprises evaporation of the solvent under a flow of an inert gas, e.g. nitrogen gas.

In some embodiments, a method for preparing a salt compound of the general formula A comprises a step of adding a suitable acid to a solution or slurry of compound 1.

In some embodiments, a method for preparing a salt compound of the general formula A comprises a step of heating.

In certain embodiments, a salt compound of formula A precipitates from the mixture. In another embodiment, a salt compound of formula A crystallizes from the mixture. In other embodiments, a salt compound of formula A crystallizes from solution following seeding of the solution (i.e., adding crystals of a salt compound of formula A to the solution).

A salt compound of formula A can precipitate out of the reaction mixture, or be generated by removal of part or all of the solvent through methods such as evaporation, distillation, filtration (ex. nanofiltration, ultrafiltration), reverse osmosis, absorption and reaction, by adding an anti-solvent such as heptane, by cooling or by different combinations of these methods.

As described generally above, a salt compound of formula A is optionally isolated. It will be appreciated that a salt compound of formula A may be isolated by any suitable physical means known to one of ordinary skill in the art. In certain embodiments, precipitated solid salt compound of formula A is separated from the supernatant by filtration. In other embodiments, precipitated solid salt compound of formula A is separated from the supernatant by decanting the supernatant.

In certain embodiments, a salt compound of formula A is separated from the supernatant by filtration.

In certain embodiments, an isolated salt compound of formula A is dried in air. In other embodiments an isolated salt compound of formula A is dried under reduced pressure, optionally at elevated temperature.

### Uses of Compounds and Pharmaceutically Acceptable Compositions

As described generally above, compound 1, and pharmaceutically acceptable solid forms and salts thereof described herein, are adrenergic receptor modulating compounds (e.g., an agonist, partial agonist or antagonist of an adrenergic receptor). The adrenergic receptor modulating compounds of the present disclosure can in some embodiments find use in modulating the activity of a target adrenergic receptor in vitro or in vivo. Aspects of the subject methods include contacting a sample with an effective amount of an adrenergic receptor modulating compound (e.g., as described herein) to determine whether the desired activity exists.

Adrenergic receptors (ADRs) are G-protein coupled receptors (GPCR) that are widely expressed throughout the body and play an important role in regulating multiple physiological processes including cognition, stress-related behavior, inflammation, and smooth muscle contraction/dilation, cardiac muscle contraction, airway reactivity and cognition. Adrenergic receptors mediate the central and peripheral effects of noradrenaline (NA) and adrenaline. Multiple subtypes of ADRs exist, including α-adrenergic receptors and β-adrenergic receptors. Each subtype is expressed in distinct patterns and involved in different physiological processes. Therefore, ligands that selectively target one subtype are valuable both as research tools to identify the roles of different ADR subtypes and as therapeutic agents for multiple diseases related to dysfunction of the NA and adrenaline systems.

β-adrenergic receptors further include three sub-types: β1-adrenergic receptor (β1-ADR), β2-adrenergic receptor (β2-ADR), and β3-adrenergic receptor (β3-ADR). Because these subtypes are expressed in distinct patterns and involved in different physiological processes, ligands that can selectively target one subtype have therapeutic potential for multiple diseases. However, discovery of subtype-selective ligands has been challenging due to a high level of sequence homology shared by these subtypes. A lot of existing agonists for β-adrenergic receptors also exhibit inferior blood-brain-barrier (BBB) penetration, which is required in an effort for drug discovery for central nervous system (CNS) indications.

As a class of G-protein coupled receptor, adrenergic receptors signal via G protein-and β-arrestin-dependent pathways. G protein- or β-arrestin signaling can mediate different physiological responses. Recently, it has become clear that agonists can show biased activation of signaling pathways. The ability of ligands to activate the receptor and produce responses in a pathway-dependent manner has been termed “signaling bias” or “functional selectivity”. As G proteins and β-arrestins mediate distinct physiological processes, biased agonists can provide improved therapeutic selectivity with reduced adverse effects. Thus, the present disclosure is directed to β-adrenergic receptor subtype-selective agonists with improved blood-brain-barrier (BBB) penetration.

In certain embodiments a compound as disclosed herein is an agonist, partial agonist or antagonist of an adrenergic receptor; in some embodiments the compound is a β1-adrenergic receptor agonist, β2-adrenertic receptor agonist or non-selective β1/β2-adrenergic receptor agonist; in some embodiments the compound is a β1-adrenergic receptor agonist; in some embodiments the compound is a β2-adrenergic receptor agonist; in some embodiments the compound is a compound is a non-selective β1/β2-adrenergic agonist.

An adrenergic receptor modulating compound can be an agonist of the target adrenergic receptor. In some cases, an effective amount of an adrenergic receptor modulating compound is an amount sufficient to activate an activity related to the adrenergic receptor in a cell by 10% or more, such as 20% or more, 30% or more, 40% or more, 50% or more, 60% or more, 70% or more, 80% or more, 90% or more, 100% or more, 200% or even more relative to a control, e.g., a control cell exhibiting a known activity level of the receptor.

The adrenergic receptor modulating compound can be a partial agonist of the target adrenergic receptor. In some cases, an effective amount of an adrenergic receptor modulating compound is an amount sufficient to achieve partially agonism of the adrenergic receptor in a cell, e.g., where the subject compound achieves 10% activation or more of the receptor, such as 20% or more, 30% or more, 40% or more, 50% or more, 60% or more, 70% or more, 80% or more, or 90% or more, relative to a control, e.g., a receptor that is fully activated. Partial agonism may be assessed using any convenient methods, such as a cell-based assay using a known full agonist as a 100% activation control, where the relative maximum activation of the receptor can be measured relative to the full agonist.

The adrenergic receptor modulating compound can be an antagonist of the target adrenergic receptor. In some cases, an effective amount of an adrenergic receptor modulating compound is an amount sufficient to inhibit or decrease the activity of the target adrenergic receptor in a sample by 10% or more, such as 20% or more, 30% or more, 40% or more, 50% or more, 60% or more, 70% or more, 80% or more, 90% or more, or even more relative to a control, e.g., a sample not contacted with the compound of interest.

In some embodiments, a compound of the present disclosure acts as a low nM partial agonist of the β2 adrenergic receptor. For instance, in some embodiments, a compound of the present disclosure has an EC₅₀ of less than about 1 nM, less than about 5 nM, less than about 10 nM, less than about 15 nM, less than about 20 nM, less than 25 nM, less than 30 nM, less than 35 nM, less than 40 nM, less than 45 nM, less than 50 nM, less than 55 nM, less than 60 nM, less than 65 nM, less than 70 nM, less than 75 nM, less than 80 nM, less than 85 nM, less than 90 nM, less than 95 nM, or less than 100 nM. In some embodiments, a compound of the present disclosure acts as a low nM partial agonist of the β2 adrenergic receptor and has an EC₅₀ of from about 0.001 nM to about 200 nM, 0.001 nM to about 150 nM, about 0.001 nM to about 100 nM, 0.01 nM to about 100 nM, 0.1 nM to about 100 nM, or about 0.1 nM to about 80 nM, or about 0.1 nM to about 60 nM, or about 0.1 nM to about 40 nM, or about 0.1 nM to about 30 nM, or about 0.1 nM to about 20 nM, or about 0.1 nM to about 10 nM.

In some embodiments, a compound of the present disclosure acts as a low µM partial agonist of the β2 adrenergic receptor. For instance, in some embodiments, a compound of the present disclosure has an EC₅₀ of less than about 0.1 µM, less than about 0.5 µM, less than about 1.0 µM, less than about 1.5 µM, less than about 2.0 µM, less than about 2.5 µM, less than about 3.0 µM, less than about 3.5 µM, less than about 4.0 µM, less than about 4.5 µM, less than about 5.0 µM, less than about 5.5 µM, less than about 6.0 µM, less than about 6.5 µM, less than about 7.0 µM, less than about 7.5 µM, less than about 8.0 µM, less than about 8.5 µM, less than about 9.0 µM, less than about 9.5 µM, or less than about 10.0 µM,

In some embodiments, a compound of the present disclosure acts as a low µM partial agonist of the β2 adrenergic receptor and has an EC₅₀ of from about 0.01 µM to about 10 µM, about 0.01 µM to about 9.0 µM, about 0.01 µM to about 8.0 µM, about 0.01 µM to about 7.0 µM, about 0.01 µM to about 6.0 µM, about 0.01 µM to about 5.0 µM, about 0.01 µM to about 4.0 µM, about 0.01 µM to about 3.0 µM, about 0.01 µM to about 2.0 µM, about 0.01 µM to about 1.0 µM, about 0.01 µM to about 9.0 µM, about 0.1 µM to about 1.0 µM,

In some embodiments of the method, the target adrenergic receptor is a β1-adrenergic receptor. In some embodiments of the method, the target adrenergic receptor is a β2-adrenergic receptor. In some embodiments of the method, the target adrenergic receptor is a β3-adrenergic receptor. In some embodiments, the compound is an agonist for both β1-adrenergic receptor and β2-adrenergic receptor. In certain cases, the compound is selective for the β2-adrenergic receptor over a β1-adrenergic receptor.

The target adrenergic receptor may be one that is responsible for a mediating an intracellular signal or pathway in a cell. In some embodiments, the sample includes a cell and modulating the adrenergic receptor modulates a physiological process in the cell. Any convenient physiological processes can be targeted for modulation in a cell using the subject methods. In some embodiments, the physiological process is one that is implicated in cardiac function, in certain instances, the physiological process is one that is implicated in cognitive function. In certain instances, the physiological process is one that is implicated in an inflammatory pathway or condition. The subject methods can provide for mediation of the intracellular concentration of a signaling molecule in a cell, such as cAMP. The subject methods can provide for partial or full blockage of the target adrenergic receptor to result in modulation (e.g., activation) of cAMP in a sample. In some embodiments, the method does not modulate β-arrestin pathways of the cell. In some cases, the cells are inflammatory cells and the function of the cells is regulated. The subject methods can provide for inhibition of an inflammatory pathway in a cell. In some cases, TNF-alpha is inhibited in the cell, e.g., the concentration or production of TNF-alpha is reduced by practicing the subject method. In certain embodiments of the method, the cell is a neuron. In some embodiments, modulating the adrenergic receptor enhances neurogenesis.

Further disclosed is a method of treating a subject with a disease, the method comprising administering to the subject a therapeutically effective amount of a compound as disclosed herein, i.e., a compound selected from compounds 1, 2, 3, 4, 5, 6, 7, 8, 9, 10, 11, 12, 13, and 14 and any polymorphic forms thereof. In some embodiments, the disease is a β1-adrenergic receptor or β2-adrenergic receptor mediated disease or disorder in a patient.

In some embodiments, the disease is selected from myocardial infarction, stroke, ischemia, Alzheimer’s disease, Parkinson’s disease, Gehrig’s disease (Amyotrophic Lateral Sclerosis), Huntington’s disease, Multiple Sclerosis, senile dementia, subcortical dementia, arteriosclerotic dementia, AIDS-associated dementia, other dementias, cerebral vasculitis, epilepsy, Tourette’s syndrome, Wilson’s disease, Pick’s disease, encephalitis, encephalomyelitis, meningitis, prion diseases, cerebellar ataxias, cerebellar degeneration, spinocerebellar degeneration syndromes, Friedrich’s ataxia, ataxia telangiectasia, spinal dysmyotrophy, progressive supranuclear palsy, dystonia, muscle spasticity, tremor, retinitis pigmentosa, striatonigral degeneration, mitochondrial encephalomyopathies, and neuronal ceroid lipofuscinosis. In some embodiments, the compound is administered to the subject through oral, enteral, topical, inhalation, transmucosal, intravenous, intramuscular, intraperitoneal, subcutaneous, intranasal, epidural, intracerebral, intracerebroventricular, epicutaneous, extra-amniotic, intra-arterial, intra-articular, intracardiac, intracavernous, intradermal, intralesional, intraocular, intraosseous infusion, intraperitoneal, intrathecal, intrauterine, intravaginal, intravesical, intravitreal, transdermal, perivascular, buccal, vaginal, sublingual, or rectal route.

In some embodiments, the disease is a neurodegenerative disease that is one or more selected from the group consisting of MCI (mild cognitive impairment), aMCI (amnestic MCI), Vascular Dementia, Mixed Dementia, FTD (fronto-temporal dementia; Pick’s disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson’s disease), PDD (PD dementia), ADHD (attention deficit hyperactivity disorder), Alzheimer’s disease (AD), early AD, and Down Syndrome (DS). In some embodiments the disease is a neurodegenerative disease that is one or more selected from the group consisting of MCI, aMCI, Vascular Dementia, Mixed Dementia, FTD (fronto-temporal dementia; Pick’s disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson’s disease), PDD (PD dementia), and ADHD (attention deficit hyperactivity disorder). In some embodiments the subject does not have Alzheimer’s disease (AD). In some embodiments the subject does not have Down Syndrome.

In certain embodiments of the methods disclosed herein, the methods include administering to the subject a compound as disclosed herein and a peripherally acting β-blocker (PABRA).

As used herein, the term “peripherally acting β-blocker (PABRA)” means a β adrenergic receptor antagonist or simply a β1-, β2- or non-selective β-blocker. Examples of selective peripherally acting β-blockers (PABRA) that may in certain embodiments be used in the methods disclosed herein include nadolol, atenolol, sotalol and labetalol. In certain embodiments a β-blocker that can be used in the methods herein is one or more selected from the group consisting of acebutolol, betaxolol, bisoprolol, celiprolol, esmolol, metaprolol and nevivolol; in other embodiments the methods do not use acebutolol, betaxolol, bisoprolol, celiprolol, esmolol, metaprolol or nevivolol as a β-blocker.

In certain embodiments a peripherally acting β-blocker (PABRA) is administered to the subject prior to administration of a compound of the disclosure; in other embodiments a peripherally acting β-blocker (PABRA) is administered to the subject concurrently with the administration of a compound of the disclosure.

In certain embodiments of the compositions and methods provided herein, one or more peripherally acting β-blockers (PABRA) are administered prior to or concurrently with a compound of the disclosure in order to inhibit or preclude agonism of peripheral β1 and/or β2 adrenergic receptors by a compound of the disclosure. In various embodiments it is preferred to block peripheral β1 and/or β2 adrenergic receptors in accordance with the compositions and methods of the present disclosure in order to preclude, or at least minimize, any adverse peripheral cardiac, metabolic or muscular effects on humans being treated.

In some embodiments of the methods provided herein, a β1 agonist and or a β2 agonist, or a non-selective β1 / β2 agonist is administered to the patient in addition to a compound as disclosed herein.

As used herein, the term “β1 agonist” is used to mean β1-adrenergic receptor agonist or β1-ADR agonist. In certain embodiments the term β1 agonist is understood to include compounds that are primarily β1 agonists, but which may also exhibit some peripheral agonism for other adrenergic receptors, such as β2-adrenergic receptors. In this application, the terms “β1-adrenergic receptor agonist”, “β1-ADR agonist”, “β1AR agonist” and “β1 agonist” may be used interchangeably. In certain embodiments, the term β1-ADR agonist expressly includes both selective and partial agonists, as well as biased and non-biased agonists. Examples of β1 adrenergic agonists include, for example, xamoterol, noradrenalin, isoprenaline, dopamine, pindolol and dobutamine and the pharmaceutically acceptable salts of any of the above. Partial agonists and ligands of the β1-ADR are known. Further, using the methodology of Kolb et al, but for β1-ADR instead, one skilled in the art could determine new ligands by structure-based discovery. See Proc. Natl. Acad. Sci. USA 2009, 106, 6843-648.

As used herein, the term β2 agonist is used to mean β2-adrenergic receptor agonist or β2-ADR agonist. In certain embodiments, the term β2 agonist is understood to include compounds that are primarily β2 agonists, but which may also exhibit some peripheral agonism for other adrenergic receptors, such as β1-adrenergic receptors. In this application the terms “β2-adrenergic receptor agonist”, “β2-ADR agonist”, “β2AR agonist” and “β2 agonist” may be used interchangeably. In some embodiments the term β2-ADR agonist expressly includes both selective and partial agonists. β2 agonists that may be used in accordance with various aspects and embodiments of the present disclosure may be short-acting, long-acting or ultra long-acting. Examples of short-acting β2 agonists that may be used are salbutamol, levosalbutamol, terbutaline, pirbuterol, procaterol, metaproterenol, bitolterol mesylate, oritodrine, isoprenaline, salmefamol, fenoterol, terbutaline, albuterol, and isoetharine. Examples of long-acting β2 agonists that may be used are salmeterol, bambuterol, formoterol and clenbuterol. Examples of ultra long-acting β2 agonists include indacaterol, vilanterol and olodaterol.

As used herein, the terms “combination,” “combined,” and related terms refer to the simultaneous or sequential administration of therapeutic agents in accordance with this disclosure. For example, a described compound may be administered with another therapeutic agent simultaneously or sequentially in separate unit dosage forms or together in a single unit dosage form. Accordingly, the present disclosure provides a single unit dosage form comprising a described compound, an additional therapeutic agent, and a pharmaceutically acceptable carrier, adjuvant, or vehicle. Two or more agents are typically considered to be administered “in combination” when a patient or individual is simultaneously exposed to both agents. In many embodiments, two or more agents are considered to be administered “in combination” when a patient or individual simultaneously shows therapeutically relevant levels of the agents in a particular target tissue or sample (e.g., in brain, in serum, etc.).

When the compounds of this disclosure are administered in combination therapies with other agents, they may be administered sequentially or concurrently to the patient. Alternatively, pharmaceutical or prophylactic compositions according to this disclosure comprise a combination of ivermectin, or any other compound described herein, and another therapeutic or prophylactic agent. Additional therapeutic agents that are normally administered to treat a particular disease or condition may be referred to as “agents appropriate for the disease, or condition, being treated.”

In some embodiments, the subject method includes administering a therapeutically effective amount of one or more additional active agents. By combination therapy is meant that an adrenergic receptor modulating compound can be used in a combination with another therapeutic agent to treat a single disease or condition. In particular embodiments, a compound of the present disclosure is administered concurrently with the administration of another therapeutic agent, which can be administered as a component of a composition including the compound of the present disclosure or as a component of a different composition.

The subject compounds can be administered in combination with other therapeutic agents in a variety of therapeutic applications. Therapeutic applications of interest for combination therapy include those applications in which activity of a target adrenergic receptor is the cause or a compounding factor in disease progression. As such, the subject compounds find use in combination therapies in which the inhibition of a target adrenergic receptor in the subject is desired. Examples of disease conditions which may be treated by a combination therapy including a subject compound include, but are not limited to, cardiac conditions or diseases, neurodegenerative or neurodevelopmental disease, respiratory disorders, asthma, memory impairment, depression, inflammatory diseases, stroke, ischemic brain or tissue injury and cancer. Agents of interest which can be used in jointly with the subject adrenergic receptor modulating compounds include, but are not limited to, antidepressants, antipsychotics, beta-blockers, vasoconstrictors, antihypotensives, decongestants, chemotherapeutic agents, agents used in Alzheimer’s disease, and anti-inflammatory agents.

The subject adrenergic receptor modulating compounds can be used jointly with any agent useful in the treatment of a cardiac condition, such as cardiogenic shock, hypertension, congestive heart failure, coronary heart disease, arrhythmias, myocardial infarction or ischemic heart diseases. Agents of interest which can be used in jointly with the subject adrenergic receptor modulating compounds include, but are not limited to, denopamine, dobutamine, xamoterol, acebutolol, atenolol, betaxolol, bisoprolol, pindolol, esmolol, metoprolol, nebivolol, vortioxetine, Carvedilol, Labetalol, Phentolamine, Prazosin, Cirazoline, Methoxamine, Synephrine, Etilefrine, Metaraminol, Midodrine, and cumarin.

The subject adrenergic receptor modulating compounds can be used jointly with any agent useful in the treatment of a neurodegenerative or neurodevelopmental disease, such as such as Alzheimer’s Disease, memory impairment, cognitive impairment, depression, stroke and ischemic brain or tissue injury, Down’s syndrome or Autism. Agents of interest which can be used in jointly with the subject adrenergic receptor modulating compounds include, but are not limited to, acepromazine. In some embodiments, the subject adrenergic receptor modulating compounds can be used in the treatment of a disease, such as a neurodegenerative or neurodevelopmental disease, in combination with a cholinesterase inhibitor or a NMDA receptor modulator. Agents of interest include, but are not limited to, Donepezil, Aricept, Galantamine, Razadyne, Memantine, Namenda, Rivastigmine, Exelon, Tacrine and Cognex. Other agents of interest which can be used in jointly with the subject adrenergic receptor modulating compounds include, but are not limited to, 4-NEMD, 7-Me-marsanidine, Agmatine, Apraclonidine, Brimonidine, Cannabigerol, Clonidine, Detomidine, Dexmedetomidine, Fadolmidine, Guanabenz, Guanfacine, Lofexidine, Marsanidine, Medetomidine, Methamphetamine, Mivazerol, Rilmenidine, Romifidine, Talipexole, Tiamenidine, Tizanidine, Tolonidine, Xylazine, Xylometazoline, Aripiprazole, Asenapine, Atipamezole, Cirazoline, Clozapine, Efaroxan, Idazoxan, Lurasidone, Melperone, Mianserin, Mirtazapine, Napitane, Olanzapine, Paliperidone, Phenoxybenzamine, Phentolamine, Piribedil, Rauwolscine, Risperidone, Rotigotine, Quetiapine, Norquetiapine, Setiptiline, Tolazoline, Yohimbine, Ziprasidone and Zotepine. Other agents of interest which can be used in jointly with the subject adrenergic receptor modulating compounds include, but are not limited to, bitolterol, fenoterol, hexoprenaline, isoprenaline or isoproterenol, levosalbutamol or levalbuterol, orciprenaline or metaproterenol, pirbuterol, procaterol, salbutamol or albuterol, terbutaline, bambuterol, clenbuterol, formoterol, salmeterol, carmoterol, indacaterol, milveterol, olodaterol, vilanterol, fenoterol, hexoprenaline, isoxsuprine, ritodrine, salbutamol or albuterol, terbutaline, zilpaterol, ICI-118,551 and butoxamine.

The compounds utilized in the compositions and methods of this disclosure may also be modified by appending appropriate functionalities to enhance selective biological properties. Such modifications are known in the art and include those, which increase biological penetration into a given biological system (e.g., blood, lymphatic system, or central nervous system), increase oral availability, increase solubility to allow administration by injection, alter metabolism and/or alter rate of excretion.

The term “treatment” is used interchangeably herein with the term “therapeutic method” and refers to both 1) therapeutic treatments or measures that cure, slow down, lessen symptoms of, and/or halt progression of a diagnosed pathologic conditions, disease or disorder, and 2) and prophylactic/ preventative measures. Those in need of treatment may include individuals already having a particular medical disease or disorder as well as those who may ultimately acquire the disorder (i.e., those at risk or needing preventive measures).

The term “subject” as used herein refers to any individual or patient to which the subject methods are performed. Generally, the subject is human, although as will be appreciated by those in the art, the subject may be an animal.

The terms “therapeutically effective amount”, “effective dose”, “therapeutically effective dose”, “effective amount,” or the like refer to the amount of a subject compound that will elicit the biological or medical response in a tissue, system, animal or human that is being sought by administering said compound. Generally, the response is either amelioration of symptoms in a patient or a desired biological outcome. In some embodiments, such amount should be sufficient to modulate an adrenergic receptor.

In some embodiments, an effective amount of an adrenergic receptor modulating compound is an amount that ranges from about 50 ng/ml to 50 pg/ml (e.g., from about 50 ng/ml to 40 pg/ml, from about 30 ng/ml to 20 pg/ml, from about 50 ng/ml to 10 µg/ml, from about 50 ng/ml to 1 µg/ml, from about 50 ng/ml to 800 ng/ml, from about 50 ng/ml to 700 ng/ml, from about 50 ng/ml to 600 ng/ml, from about 50 ng/ml to 500 ng/ml, from about 50 ng/ml to 400 ng/ml, from about 60 ng/ml to 400 ng/ml, from about 70 ng/ml to 300 ng/ml, from about 60 ng/ml to 100 ng/ml, from about 65 ng/ml to 85 ng/ml, from about 70 ng/ml to 90 ng/ml, from about 200 ng/ml to 900 ng/ml, from about 200 ng/ml to 800 ng/ml, from about 200 ng/ml to 700 ng/ml, from about 200 ng/ml to 600 ng/ml, from about 200 ng/ml to 500 ng/ml, from about 200 ng/ml to 400 ng/ml, or from about 200 ng/ml to about ng/ml).

In some embodiments, an effective amount of an adrenergic receptor modulating compound is an amount that ranges from about 10 pg to 100 mg, e.g., from about 10 pg to 50 pg, from about 50 pg to 150 pg, from about 150 pg to 250 pg, from about 250 pg to 500 pg, from about 500 pg to 750 pg, from about 750 pg to 1 ng, from about 1 ng to 10 ng, from about 10 ng to 50 ng, from about 50 ng to 150 ng, from about 150 ng to 250 ng, from about 250 ng to 500 ng, from about 500 ng to 750 ng, from about 750 ng to 1 mg, from about 1 pg to 10 pg, from about 10 pg to 50 pg, from about 50 pg to 150 pg, from about 150 pg to 250 pg, from about 250 pg to 500 pg, from about 500 pg to 750 pg, from about 750 pg to 1 mg, from about 1 mg to 50 mg, from about 1 mg to 100 mg, or from about 50 mg to 100 mg. The amount can be a single dose amount or can be a total daily amount. The total daily amount can range from about 10 pg to 100 mg, or can range from about 100 mg to 500 mg, or can range from about 500 mg to 1000 mg.

Also disclosed herein are pharmaceutical compositions including compounds as disclosed herein e.g., any one of compounds 1, 2, 3, 4, 5, 6, 7, 8, 9, 10, 11, 12, 13, 14 and any polymorphic forms thereof.

The term “pharmaceutically acceptable carrier” refers to a non-toxic carrier that may be administered to a patient, together with a compound of this disclosure, and which does not destroy the pharmacological activity thereof. Pharmaceutically acceptable carriers that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In pharmaceutical compositions comprising only the compounds described herein as the active component, methods for administering these compositions may additionally comprise the step of administering to the subject an additional agent or therapy. Such therapies include, but are not limited to, an anemia therapy, a diabetes therapy, a hypertension therapy, a cholesterol therapy, neuropharmacologic drugs, drugs modulating cardiovascular function, drugs modulating inflammation, immune function, production of blood cells; hormones and antagonists, drugs affecting gastrointestinal function, chemotherapeutics of microbial diseases, and/or chemotherapeutics of neoplastic disease. Other pharmacological therapies can include any other drug or biologic found in any drug class. For example, other drug classes can comprise allergy/cold/ENT therapies, analgesics, anesthetics, anti-inflammatories, antimicrobials, antivirals, asthma/pulmonary therapies, cardiovascular therapies, dermatology therapies, endocrine/metabolic therapies, gastrointestinal therapies, cancer therapies, immunology therapies, neurologic therapies, ophthalmic therapies, psychiatric therapies or rheumatologic therapies. Other examples of agents or therapies that can be administered with the compounds described herein include a matrix metalloprotease inhibitor, a lipoxygenase inhibitor, a cytokine antagonist, an immunosuppressant, a cytokine, a growth factor, an immunomodulator, a prostaglandin or an anti-vascular hyperproliferation compound.

The term “therapeutically effective amount” as used herein refers to the amount of active compound or pharmaceutical agent that elicits the biological or medicinal response in a tissue, system, animal, individual or human that is being sought by a researcher, veterinarian, medical doctor or other clinician, which includes one or more of the following: (1) Preventing the disease; for example, preventing a disease, condition or disorder in an individual that may be predisposed to the disease, condition or disorder but does not yet experience or display the pathology or symptomatology of the disease, (2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) Ameliorating the disease; for example, ameliorating a disease, condition or disorder in an individual that is experiencing or displaying the pathology or symptomatology of the disease, condition or disorder (i.e., reversing the pathology and/or symptomatology).

### Pharmaceutically Acceptable Compositions

The compounds and compositions, according to the method of the present disclosure, are administered using any amount and any route of administration effective for treating or lessening the severity of a disorder provided above. The exact amount required will vary from subject to subject, depending on the species, age, and general condition of the subject, the severity of the infection, the particular agent, its mode of administration, and the like. Compounds of the disclosure are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this disclosure can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the disclosure are administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer’s solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

All features of each of the aspects of the disclosure apply to all other aspects mutatis mutandis. Each of the references referred to herein, including but not limited to patents, patent applications and journal articles, is incorporated by reference herein as though fully set forth in its entirety,

In order that the disclosure described herein may be more fully understood, the following examples are set forth. It should be understood that these examples are for illustrative purposes only and are not to be construed as limiting this disclosure in any manner.

### EXAMPLES

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to all compounds and subclasses and species of each of these compounds, as described herein.

### General Procedures

List of Solvents

| | |
|---|---|
| Solvent | Abbreviation |
| Methanol | MeOH |
| Ethanol | EtOH |
| 2-Propanol | IPA |
| Isobutanol | IBA |
| 2-Butanone | MEK |
| Tetrahydrofuran | THF |
| Acetonitrile | ACN |
| tert-Butyl methyl ether | MTBE |
| Acetone | - |
| Water | - |
| Toluene | Tol |
| Ethyl acetate | EtOAc |
| Isopropyl acetate | iPrOAc |
| n-Heptane | Hept |

### Analysis Methods

### X-Ray Powder Diffraction (XRPD)

XRPD patterns were identified with an X-ray diffractometer (Bruker D8 advance). The system was equipped with LynxEye detector. Samples were scanned from 3 to 40°2θ, at a step size of 0.02°2θ. The tube voltage and current were 40 KV and 40 mA, respectively.

### Differential Scanning Calorimeter (DSC)

DSC was performed using a Discovery DSC 250 (TA Instruments, US). The sample was placed into an aluminum pin-holed hermetic pan and the weight was accurately recorded. The sample was heated at a rate of 10° C./min from 25° C. to the final temperature.

### Thermogravimetric Analysis (TGA)

TGA analysis was carried out on a Discovery TGA 55 (TA Instruments, US). The sample was placed into an open tared aluminum pan, automatically weighed, and inserted into the TGA furnace. The sample was heated at a rate of 10° C./min from ambient temperature to the final temperature.

### Dynamic Vapor Sorption (DVS)

Moisture sorption/desorption data was collected on a DVS Intrinsic (SMS, UK). The sample was placed into a tared sample chamber and automatically weighed. The sample was dried at 40° C. until the dm/dt was less than 0.002% and cooled to 25° C. The instrument parameters were set as detailed below. Step time (min): 60 min; Sample temperature: 25° C.; Cycle: Full cycle; Adsorption: 0, 10, 20, 30, 40, 50, 60, 70, 80, 90; Desorption: 80, 70, 60, 50, 40, 30, 20, 10, 0; Save Data Rate: 5 s; Total flow rate: 200 sccm; Post experiment total flow: 200 sccm.

### Polarized Light Microscopy (PLM)

Light microscopy was performed using a Polarizing Microscope ECLIPSE LV100POL (Nikon, JPN).

### Proton Nuclear Magnetic Resonance (¹H NMR)

¹H NMR was performed using Bruker Advance 300 equipped with automated sample (B-ACS 120).

### High Performance Liquid Chromatography (HPLC)

HPLC analysis was performed with an Agilent HPLC 1260 series instrument. HPLC method for solubility and stability testing is listed in Table 16.

TABLE 16

HPLC Method for Solubility and Stability Testing

| | |
|---|---|
| Instrument | Agilent 1260 series |
| Column | Eclipse Plus C18, 5.0 µm, 4.6 mm x 100 mm |
| Column Temperature | 40° C. |
| Mobile phase | A: 0.1% TFA in water |
| | B: 0.1% TFA in ACN |
| Gradient condition (% of B) | 0 min: 10% |
| | 6 min: 50% |
| | 8 min: 100% |
| Flow rate | 1.0 mL/min |
| Injection Volume | 5 µL |
| UV wavelength | 220 nm |
| Post time | 2 min |
| Diluent | ACN/water (1/1) |

Example A: General Preparation of Compound 1

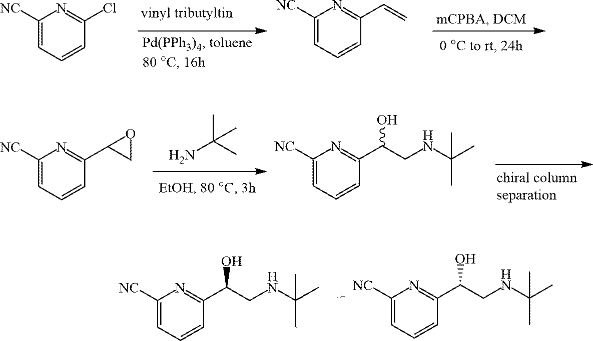

### Step 1: Synthesis of 2-cyano-6-vinylpyridine

A stirred mixture of 2-chloro-6-cyanopyridine (8.0 g, 69.3 mmol), 1-vinyltri-n-butyltin (21.97 g, 69.29 mmol, 20.34 mL), and Pd(PPh₃)₄ (3.34 g, 3.61 mmol) in anhydrous toluene (150 mL) was bubbled with N₂ for 5 min, before heating to 80° C. overnight. After cooling, the reaction mixture was poured into an aqueous solution of KF (40 g in 200 mL) and stirred for 30 min. The mixture was then filtered through celite and the solid was washed with EtOAc (2 x 50 mL). The aqueous phase of the filtrate was separated and extracted with EtOAc (2 x 250 mL). The combined organic phases were washed with brine, dried over Na₂SO₄, filtered and concentrated under reduced pressure. The residue was purified by flash chromatography eluting with Hexanes/EtOAc (silica, 95/5 to 90/10) to provide 2-cyano-6-vinylpyridine as a pale yellow liquid (6.5 g, 86%). MS (m/z): 131.1 (M+H)⁺.

### Step 2: Synthesis of 6-(oxiran-2-yl)picolinonitrile

To a stirred solution of 2-cyano-6-vinylpyridine (6.5 g, 49.94 mmol) in DCM (300 mL) was added meta-chloroperoxybenzoic acid (mCPBA) (61.56 g, 249.72 mmol) slowly portion wise at 0° C. over a period of 30 min and the mixture was stirred at RT for 24 h. After completion of the reaction, the reaction mixture was cooled to 5° C. and aqueous saturated NaHC03 solution was added and the solution was extracted with DCM (200 mL × 2). Organic layers were combined, dried over Na₂SO₄, filtered, and concentrated under reduced pressure. The residue was purified by flash chromatography eluting with Hexanes/EtOAc (silica, 90/10 to 80/20) to provide 6-(oxiran-2-yl)picolinonitrile as a colorless liquid (3.85 g, 52%). MS (m/z): 147.1 (M+H)⁺.

### Step 3: Synthesis of Compound 1

To a stirred solution of 6-(oxiran-2-yl) picolinonitrile (3.5 g, 18.2 mmol) in ethanol (25 mL) was added tert-butylamine (6.66 g, 91.0 mmol). The reaction mixture was stirred at 80° C. for 3 h in a sealed tube, while monitoring reaction by TLC and LCMS. After completion of reaction, solvent was evaporated to yield a residue, which was purified by reverse phase chromatography to provide the desired product as a racemic mixture. The racemic mixture was separated by SFC (Chiralpak AS-H (30*250) mm, 5 µ column, using CO₂: 80% Co-solvent: 20% (0.2% isopropylamine in IPA as eluent) to provide compound 1 (S)-6-(2-(tert-butylamino)-1-hydroxyethyl)picolinonitrile (1.05 g, 26.3%) and (R)-6-(2-(tert-butylamino)-1-hydroxyethyl)picolinonitrile (0.98 g, 24.5%) as white solids. Compound 1: ¹H NMR 400 MHz, DMSO-d6: δ 8.03 (t, J = 8.0 Hz, 1H), 7.90 (dd, J= 0.8 Hz, 7.6 Hz, 1H), 7.82 (d, J= 8.0 Hz, 1H), 5.63 (s, 1H), 4.60 (q, J= 4.4 Hz, 1H), 2.86-2.80 (m, 1H), 2.67-2.49 (m, 1H), 1.44-1.40 (m, 1H), 0.98 (s, 9H). (R)-6-(2-(tert-butylamino)-1-hydroxyethyl)picolinonitrile: ¹H NMR 400 MHz, DMSO-d6: 8 8.03 (t, J= 7.6 Hz, 1H), 7.90 (d, J= 6.8 Hz, 1H), 7.82 (d, J= 8.0 Hz, 1H), 5.62 (s, 1H), 4.6 (s, 1H), 2.81-2.82 (m, 1H), 2.62-2.64 (m, 1H), 1.44 (s, 1H), 0.98 (s, 9H).

### Example 1: Preparation of Free Base Forms A and B of Compound 1

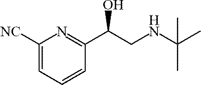

Compound 1 is prepared as described elsewhere herein.

### Form A of Compound 1

Form A of Compound 1 was prepared as described in Example A and was formed directly from the IPA/isopropylamine co-solvent as a white solid.

Characterization of the resulting material demonstrated crystalline Form A of Compound 1 free base.

Table 1, supra, is reproduced below and sets forth the X-ray diffraction peaks observed for Form A of compound 1.

TABLE 1

XRPD Peak Positions for Form A of Compound 1

| Position (°2θ) | Intensity % | Position (°2θ) | Intensity % |
|---|---|---|---|
| 6.5 | 3.1 | 23.5 | 11.8 |
| 9.3 | 100.0 | 23.9 | 3.3 |
| 9.7 | 15.8 | 25.1 | 23.1 |
| 10.2 | 7.0 | 25.6 | 6.4 |
| 11.5 | 1.4 | 25.9 | 3.7 |
| 12.6 | 20.0 | 26.2 | 1.7 |
| 13.0 | 1.5 | 27.5 | 1.6 |
| 14.7 | 2.6 | 28.3 | 11.4 |
| 15.3 | 1.5 | 29.1 | 3.9 |
| 15.7 | 3.5 | 29.6 | 6.0 |
| 16.4 | 12.8 | 30.7 | 2.4 |
| 16.9 | 21.7 | 31.1 | 2.2 |
| 17.5 | 8.6 | 32.5 | 1.1 |
| 18.8 | 20.0 | 32.9 | 1.2 |
| 19.3 | 1.9 | 34.3 | 2.8 |
| 20.0 | 2.0 | 35.6 | 0.8 |
| 20.6 | 47.0 | 36.2 | 1.2 |
| 21.7 | 1.7 | 37.2 | 2.0 |
| 22.0 | 2.5 | 38.1 | 1.6 |
| 22.5 | 6.2 | 38.6 | 0.9 |

In this and all subsequent tables, the position (°2θ) is within ± 0.2.

FIG. 1A.1 depicts an XRPD pattern of Form A of compound 1.

FIG. 1A.2 depicts a DSC thermogram and TGA trace of Form A of compound 1. The DSC thermogram of Form A of compound 1 was characterized by two endothermic peaks at about 100° C., and about 104° C.

FIG. 1A.3 depicts a ¹H NMR spectrum of Form A of compound 1.

### Form B of Compound 1

Form B of compound 1 was prepared as follows:

Procedure A: A solution of compound 1 was dissolved in THF (5 volumes) and stirred at r.t (< 25° C.). After 2 h, H₂O (5 volumes) was added followed by 3 M aqueous HCl (2 volumes). The mixture was extracted with dichloromethane (4 X 1 volume). The pH of the resultant aqueous solution was adjusted to ~9 with concentrated aqueous NH₄OH and extracted with dichloromethane (5 X 3 volumes). The combined organics were filtered and concentrated to afford Form B of compound 1 as a light brown solid.

Characterization of the resulting material demonstrated crystalline Form B of Compound 1 free base.

Table 2, supra, is reproduced below and sets forth the X-ray diffraction peaks observed for Form B of compound 1.

TABLE 2

XRPD Peak Positions for Form B of Compound 1

| Position (°2θ) | Intensity % | Position (°2θ) | Intensity % |
|---|---|---|---|
| 6.9 | 6.7 | 23.9 | 18.3 |
| 9.3 | 100.0 | 24.1 | 10.8 |
| 9.6 | 23.7 | 25.0 | 37.7 |
| 10.6 | 12.0 | 25.4 | 7.3 |
| 12.6 | 25.2 | 25.9 | 7.3 |
| 14.7 | 7.3 | 27.4 | 3.8 |
| 15.7 | 8.2 | 28.4 | 18.6 |
| 16.4 | 22.3 | 29.1 | 9.8 |
| 16.9 | 56.5 | 29.6 | 11.6 |
| 17.5 | 13.0 | 30.6 | 6.8 |
| 18.1 | 5.0 | 31.6 | 3.6 |
| 18.8 | 34.2 | 32.4 | 3.9 |
| 19.3 | 9.5 | 34.3 | 7.0 |
| 19.9 | 6.8 | 36.1 | 3.6 |
| 20.6 | 68.2 | 37.3 | 4.9 |
| 22.0 | 6.6 | 38.3 | 5.0 |
| 22.4 | 18.3 | 38.5 | 4.4 |
| 23.5 | 24.3 | | |

In this and all subsequent tables, the position (°2θ) is within ± 0.2.

FIG. 1B.1 depicts an XRPD pattern of Form B of compound 1.

FIG. 1B.2 depicts a DSC thermogram and TGA trace of Form B of compound 1. The DSC thermogram of Form B of compound 1 was characterized by an endothermic peak at about 100° C.

FIG. 1B.3 depicts a ¹H NMR spectrum of Form B of compound 1.

### Example 2: Preparation of Form A of Compound 2

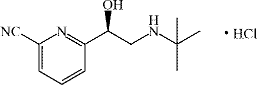

### Form A of Compound 2

Form A of compound 2 was prepared as follows:

Procedure A: Compound 1 (3.5 g) was dissolved in 35 mL IPA at 50° C. Hydrochloric acid (1.05 eq) was added. The resulting mixture was cooled to rt. After 2 h he solid was collected by filtration and dried under vacuum at 50° C. for 2 h to yield Compound 2 Form A (3.28 g, 80% yield, ~100% purity, 0.16% IPA).

Procedure B: Compound 1 (20.0 mg) was dissolved in IPA (15 V) at 50° C. The solution was allowed to cool to rt. Hydrochloric acid (1.05 eq) was added at rt. The resulting mixture was stirred for 2 h. The solid was collected by filtration and dried under vacuum at 50° C. for 2 h to yield Compound 2 Form A (71% yield, ~100% purity, 0.31% IPA).

Procedure C: Compound 1 (20.0 mg) was dissolved in IPA (10 V) at 50° C. Hydrochloric acid (1.05 eq) was added at 50° C. The resulting mixture was stirred at 50° C. for 0.5 h, and then allowed to warm to rt. After 1.5 he solid was collected by filtration and dried under vacuum at 50° C. for 2 h to yield Compound 2 Form A (77% yield, ~100% purity, 0.12% IPA).

Procedure D: Compound 1 (20.0 mg) was added to IPA (10 V) at rt. Hydrochloric acid (1.05 eq) was added. The resulting slurry was allowed to stir for 2 h. The solid was collected by filtration and dried under vacuum at 50° C. for 2 h to yield Compound 2 Form A (76% yield, ~100% purity, 0.20% IPA).

Procedure E: Compound 1 (20.0 mg) was mostly dissolved in EtOH/EtOAc (1:3; 10 V) at rt. Hydrochloric acid (1.05 eq) was added. The resulting mixture was stirred at rt for 2 h. The solid was collected by filtration and dried under vacuum at 50° C. for 2 h to yield Compound 2 Form A (70% yield, ~100% purity, negligible solvent).

Procedure F: Compound 1 was added to EtOAc (15 V) at rt. Hydrochloric acid (1.05 eq) was added. The resulting slurry was stirred for 4 h. The solid was collected by filtration to yield Compound 2 Form A.

Procedure G: Compound 1 (198.1 mg) was added to EtOAc (4 mL) at rt. Hydrochloric acid (1.05 eq) was added and the resulting precipitate was stirred at rt for 3 h. The solid was collected by filtration at 50° C. for 1 day to yield Compound 2 Form A.

Procedure H: Compound 1 (4 g) was dissolved in EtOAc (8 V). D-mandelic acid (2.4 g) was then added to the solution. The mixture was stirred for 10 min at rt. Heptane (25 V) was added to the mixture at rt and the suspension was stirred at 50° C. for 2 h and then at rt for an additional 1.5 h. The resulting solid was collected by filtration and dried under vacuum at 50° C. overnight to yield Compound 11 (5.8 g, 91% yield). The resulting compound 11 was added to EtOAc (2.58 V) and mixed to make a suspension. Concentrated HCl (1.2 mL) was added and the suspension became clear. The solution was stirred for 10 min at rt and MTBE (25.8 V) and Compound 2 Form A seed (10.12 mg) was added. Solid precipitated immediately. The suspension was stirred at rt for 3 h. The resulting solid was collected by filtration and dried under vacuum at 50° C. overnight. The solid was added to an EtOAc/MTBE solvent mixture (0.88 vol EtOAc + 7.92 vol MTBE) and stirred at rt for 10 min. The solid was filtered and dried under vacuum at 50° C. for 3 h to yield Compound 2 Form A (2.23 g, 53% yield, 99.9% purity).

Characterization of the resulting materials demonstrated anhydrous crystalline Form A of Compound 2.

Table 3, supra, is reproduced below and sets forth the X-ray diffraction peaks observed for Form A of compound 2.

TABLE 3

XRPD Peak Positions for Form A of Compound 2

| Position (°2θ) | Intensity % | Position (°2θ) | Intensity % |
|---|---|---|---|
| 6.9 | 12.4 | 27.8 | 6.7 |
| 10.6 | 100.0 | 28.0 | 29.0 |
| 12.6 | 1.8 | 28.4 | 2.1 |
| 13.9 | 3.6 | 29.4 | 2.6 |
| 15.6 | 19.7 | 30.4 | 1.7 |
| 17.7 | 2.5 | 31.6 | 5.5 |
| 19.3 | 59.9 | 32.2 | 18.1 |
| 19.9 | 8.5 | 34.6 | 2.2 |
| 21.0 | 8.6 | 35.4 | 2.5 |
| 21.3 | 6.0 | 36.3 | 1.6 |
| 23.8 | 83.2 | 36.8 | 1.3 |
| 25.4 | 6.9 | 37.4 | 3.6 |
| 25.6 | 4.4 | 38.3 | 2.3 |
| 26.6 | 2.0 | 39.3 | 2.5 |

In this and all subsequent tables, the position (°2θ) is within ± 0.2.

FIG. 2A.1 depicts an XRPD pattern of Form A of compound 2.

FIG. 2A.2 depicts a DSC thermogram and TGA trace of Form A of compound 2. The TGA trace showed almost no weight loss before decomposition. The DSC thermogram of Form A of compound 2 was characterized by an endothermic peak at about 215° C.

FIGS. 2A.3 and 2A.4 depict DVS plots of Form A of compound 2. The DVS results showed that Form A was slightly hygroscopic with 0.58% water uptake at 80%RH. The XRPD pattern remained unchanged after DVS testing.

Ion chromatography analysis of Form A of compound 2 determined an HCl content of about 13.5%, indicating that Form A of compound 2 is a mono-HCl salt (14.26% theoretical).

### Example 3: Preparation of Form A of Compound 3

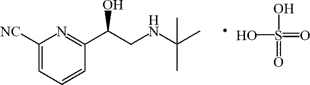

### Form A of Compound 3

Form A of compound 3 was prepared as follows:

Procedure A: Compound 1 (20.4 mg) was dissolved in iPrOAc (20 V) at rt. Sulfuric acid (0.6 eq) was added. Solid rapidly precipitated. The solid was collected by filtration and dried under vacuum to yield Compound 3 Form A.

Characterization of the resulting material demonstrated anhydrous crystalline Form A of Compound 3, with no residual organic solvent observed by NMR.

Table 4, supra, is reproduced below and sets forth the X-ray diffraction peaks observed for Form A of compound 3.

TABLE 4

XRPD Peak Positions for Form A of Compound 3

| Position (°2θ) | Intensity % | Position (°2θ) | Intensity % |
|---|---|---|---|
| 6.4 | 60.8 | 25.8 | 5.0 |
| 10.2 | 6.0 | 26.4 | 5.1 |
| 11.1 | 100.0 | 26.9 | 8.2 |
| 12.2 | 5.8 | 27.9 | 6.6 |
| 13.3 | 12.7 | 28.9 | 5.7 |
| 15.8 | 15.9 | 30.5 | 4.9 |
| 16.1 | 37.7 | 31.0 | 5.3 |
| 17.8 | 18.7 | 31.4 | 4.7 |
| 18.4 | 35.7 | 31.7 | 5.8 |
| 19.3 | 5.4 | 31.9 | 7.4 |
| 21.3 | 12.4 | 32.4 | 6.0 |
| 21.9 | 32.7 | 33.6 | 7.4 |
| 22.7 | 39.1 | 34.9 | 4.6 |
| 23.4 | 15.5 | 35.4 | 4.1 |
| 23.8 | 73.9 | 36.8 | 5.3 |
| 24.7 | 13.3 | 37.4 | 3.8 |

In this and all subsequent tables, the position (°2θ) is within ± 0.2.

FIG. 3A.1 depicts an XRPD pattern of Form A of compound 3.

FIG. 3A.2 depicts a DSC thermogram and TGA trace of Form A of compound 3. The TGA trace showed almost no weight loss before decomposition. The DSC thermogram of Form A of compound 3 was characterized by an endothermic peak at about 247° C.

### Example 4: Preparation of Form A of Compound 4

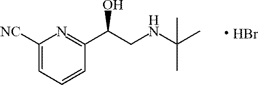

### Form A of Compound 4

Form A of compound 4 was prepared as follows:

Procedure A: Compound 1 was dissolved in EtOAc (20 V) at rt. HBr acid (1.05 eq) was added resulting in a precipitate. The solid was collected by filtration and dried under vacuum at 50° C. to yield Compound 4 Form A.

Characterization of the resulting material demonstrated anhydrous crystalline Form A of Compound 4, with no residual organic solvent observed by NMR.

Table 5, supra, is reproduced below and sets forth the X-ray diffraction peaks observed for Form A of compound 4.

TABLE 5

XRPD Peak Positions for Form A of Compound 4

| Position (°2θ) | Intensity % | Position (°2θ) | Intensity % |
|---|---|---|---|
| 6.8 | 42.7 | 28.9 | 3.2 |
| 10.6 | 100.0 | 30.1 | 3.7 |
| 11.5 | 11.5 | 30.4 | 3.4 |
| 13.6 | 5.9 | 30.9 | 11.7 |
| 15.6 | 31.1 | 31.3 | 6.3 |
| 17.5 | 5.6 | 31.6 | 6.1 |
| 18.8 | 40.0 | 32.1 | 11.7 |
| 19.5 | 10.7 | 32.5 | 2.8 |
| 20.5 | 2.4 | 34.1 | 3.4 |
| 21.1 | 2.6 | 34.5 | 6.0 |
| 21.3 | 8.4 | 35.2 | 3.3 |
| 23.5 | 55.9 | 35.5 | 4.0 |
| 24.9 | 13.5 | 36.7 | 2.6 |
| 25.4 | 15.2 | 37.1 | 4.3 |
| 26.8 | 4.7 | 37.4 | 5.1 |
| 27.4 | 48.2 | 38.2 | 5.0 |
| 28.1 | 4.8 | 39.2 | 1.6 |
| 28.6 | 10.0 | 39.7 | 2.1 |

In this and all subsequent tables, the position (°2θ) is within ± 0.2.

FIG. 4A.1 depicts an XRPD pattern of Form A of compound 4.

FIG. 4A.2 depicts a DSC thermogram and TGA trace of Form A of compound 4. The TGA trace showed no weight loss before decomposition. The DSC thermogram of Form A of compound 4 was characterized by an endothermic peak at about 173° C.

### Example 5: Preparation of Form A of Compound 5

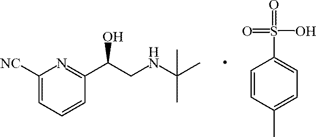

### Form A of Compound 5

Form A of compound 5 was prepared as follows:

Procedure A: Compound 1 (20.2 mg) was added to iPrOAc (20 V) at rt with stirring. P-toluenesulfonic acid (1.05 eq) was added resulting in a slurry. The solid was collected by filtration and dried under vacuum to yield Compound 5 Form A.

Characterization of the resulting material demonstrated anhydrous crystalline Form A of Compound 5, with no residual organic solvent observed by NMR. The ratio of tosylate to compound 1 in Form A of compound 5 was determined to be 1:1 by ¹H NMR analysis.

Table 6, supra, is reproduced below and sets forth the X-ray diffraction peaks observed for Form A of compound 5.

TABLE 6

XRPD Peak Positions for Form A of Compound 5

| Position (°2θ) | Intensity % | Position (°2θ) | Intensity % |
|---|---|---|---|
| 3.2 | 8.2 | 23.8 | 5.6 |
| 7.1 | 100.0 | 24.3 | 2.7 |
| 7.6 | 25.5 | 24.9 | 5.2 |
| 9.9 | 10.0 | 25.1 | 12.9 |
| 13.6 | 4.6 | 25.7 | 2.9 |
| 14.1 | 11.8 | 26.6 | 6.0 |
| 15.4 | 24.5 | 27.6 | 7.4 |
| 15.9 | 9.8 | 27.8 | 6.0 |
| 17.0 | 15.6 | 28.6 | 1.7 |
| 17.4 | 3.1 | 28.8 | 2.2 |
| 18.8 | 4.0 | 29.8 | 5.4 |
| 19.5 | 6.5 | 31.2 | 2.6 |
| 19.9 | 28.0 | 33.1 | 2.5 |
| 20.8 | 9.4 | 34.4 | 1.9 |
| 21.1 | 11.1 | 35.1 | 2.8 |
| 21.8 | 7.2 | 36.3 | 4.0 |
| 22.3 | 3.5 | 37.1 | 1.2 |
| 22.7 | 4.7 | 38.1 | 1.8 |
| 23.3 | 31.9 | 38.5 | 2.3 |

In this and all subsequent tables, the position (°2θ) is within ± 0.2.

FIG. 5A.1 depicts an XRPD pattern of Form A of compound 5.

FIG. 5A.2 depicts a DSC thermogram and TGA trace of Form A of compound 5. The TGA trace showed almost no weight loss before decomposition. The DSC thermogram of Form A of compound 5 was characterized by an endothermic peak at about 139° C.

FIG. 5A.3 depicts a ¹H NMR spectrum of Form A of compound 5.

FIGS. 5A.4 and 5A.5 depict DVS plots of Form A of compound 5. The DVS plot showed that Form A of compound 5 was slightly hygroscopic with 1.25% and 2.77% water uptake at 80%RH and 90%RH, respectively. The XRPD pattern of the tested sample remained unchanged after DVS testing.

### Example 6: Preparation of Form A of Compound 6

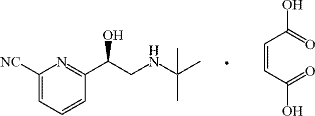

### Form A of Compound 6

Form A of compound 6 was prepared as follows:

Procedure A: Compound 1 (20 mg) was dissolved in EtOAc (15 V) at rt. Solid maleic acid (1.05 eq) was added. The solution was stirred for 4 hours, during which a solid precipitated. The solid was collected by filtration and dried under vacuum to yield Compound 6 Form A.

Procedure B: Compound 1 (20 mg) was dissolved in EtOAc (20 V) at rt. A solution of maleic acid (1 M in MeOH, 1.05 eq) was added. The solution was stirred for 3 hours. MTBE (150 V) was added and the solution was stirred for 1 additional hour. The resulting precipitate was collected by filtration and dried under vacuum to yield Compound 6 Form A.

Procedure C: Compound 1 (20 mg) was dissolved in EtOAc (20 V) at 50° C. A solution of maleic acid (1 M in MeOH, 1.0 eq) was added. The solution was stirred for 1 hour. MTBE (150 V) was added and the solution was stirred at 50° C. for 1 day. The resulting precipitate was collected by filtration and dried under vacuum to yield Compound 6 Form A.

Procedure D: Compound 1 (20 mg) was dissolved in EtOAc (20 V) at 50° C. A solution of maleic acid (1 M in MeOH, 0.55 eq) was added. The solution was stirred for 1 hour, MTBE (150 V) was added and the solution was stirred at 50° C. for 1 day. The resulting precipitate was collected by filtration and dried under vacuum to yield Compound 6 Form A.

Characterization of the resulting material demonstrated anhydrous crystalline Form A of Compound 6, with no residual organic solvent observed by ¹H NMR. The ratio of maleate to compound 1 in Form A was determined to be 1:1 based on ¹H NMR analysis.

Table 7, supra, is reproduced below and sets forth the X-ray diffraction peaks observed for Form A of compound 6.

TABLE 7

XRPD Peak Positions for Form A of Compound 6

| Position (°2θ) | Intensity % | Position (°2θ) | Intensity % |
|---|---|---|---|
| 6.4 | 2.7 | 24.7 | 2.8 |
| 7.7 | 100.0 | 25.6 | 4.4 |
| 8.6 | 22.1 | 26.0 | 28.2 |
| 10.2 | 4.2 | 26.3 | 17.2 |
| 10.8 | 40.7 | 27.4 | 20.2 |
| 12.9 | 7.2 | 29.1 | 5.4 |
| 14.0 | 1.4 | 30.0 | 3.4 |
| 14.5 | 12.9 | 30.8 | 3.0 |
| 15.1 | 4.6 | 31.1 | 4.8 |
| 15.5 | 7.5 | 32.8 | 2.3 |
| 16.4 | 5.0 | 33.2 | 2.0 |
| 17.4 | 5.7 | 33.8 | 2.2 |
| 18.0 | 19.5 | 34.1 | 3.4 |
| 18.5 | 9.1 | 34.7 | 2.5 |
| 19.5 | 2.2 | 35.3 | 5.7 |
| 19.9 | 16.1 | 36.1 | 1.6 |
| 20.5 | 4.2 | 36.6 | 2.0 |
| 21.3 | 2.7 | 37.2 | 1.6 |
| 22.2 | 2.1 | 37.8 | 1.1 |
| 22.7 | 8.4 | 38.3 | 2.3 |
| 23.4 | 16.6 | 38.8 | 1.9 |
| 24.3 | 5.4 | 39.6 | 2.0 |

In this and all subsequent tables, the position (°2θ) is within ± 0.2.

FIG. 6A.1 depicts an XRPD pattern of Form A of compound 6.

FIG. 6A.2 depicts a DSC thermogram and TGA trace of Form A of compound 6. The TGA trace showed almost no weight loss before decomposition. The DSC thermogram of Form A of compound 6 was characterized by two endothermic peaks at about 132° C., and about 146° C.

FIG. 6A.3 depicts a ¹H NMR spectrum of Form A of compound 6.

### Example 7: Preparation of Form A of Compound 7

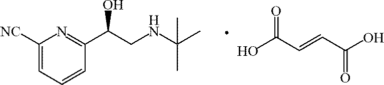

### Form A of Compound 7

Form A of compound 7 was prepared as follows:

Procedure A: Compound 1 (20.3 mg) was dissolved in EtOH (20 V) at rt. Fumaric acid (1.05 eq) was added. The solution was stirred at rt for 3 hours. MTBE (250 V) was added and the solution was stirred at 50° C. for 1 day. The resulting precipitate was collected by filtration and dried under vacuum at 50° C. to yield Compound 7 Form A.

Characterization of the resulting material demonstrated anhydrous crystalline Form A of Compound 7, with no residual organic solvent observed by ¹H NMR. The ratio of fumarate to compound 1 in Form A was determined to be 1:1 based on ¹H NMR analysis.

Table 8, supra, is reproduced below and sets forth the X-ray diffraction peaks observed for Form A of compound 7.

TABLE 8

XRPD Peak Positions for Form A of Compound 7

| Position (°2θ) | Intensity % | Position (°2θ) | Intensity % |
|---|---|---|---|
| 4.9 | 11.3 | 17.5 | 11.3 |
| 5.5 | 14.7 | 18.3 | 4.1 |
| 5.8 | 12.0 | 19.5 | 6.4 |
| 7.4 | 15.5 | 21.4 | 4.3 |
| 10.1 | 15.7 | 22.6 | 12.1 |
| 11.1 | 100.0 | 23.0 | 34.5 |
| 12.2 | 6.3 | 23.9 | 8.4 |
| 12.8 | 4.7 | 24.6 | 43.3 |
| 14.0 | 5.5 | 26.4 | 13.3 |
| 15.0 | 6.6 | 27.1 | 11.3 |
| 15.7 | 6.4 | 28.2 | 11.7 |
| 16.0 | 7.1 | 30.8 | 4.7 |
| 16.7 | 5.1 | 35.5 | 3.1 |

In this and all subsequent tables, the position (°2θ) is within ± 0.2.

FIG. 7A.1 depicts an XRPD pattern of Form A of compound 7.

FIG. 7A.2 depicts a DSC thermogram and TGA trace of Form A of compound 7. The TGA trace showed almost no weight loss before decomposition. The DSC thermogram of Form A of compound 7 was characterized by two endothermic peaks at about 138° C., and about 155° C.

FIG. 7A.3 depicts a ¹H NMR spectrum of Form A of compound 7.

### Example 8: Preparation of Form A of Compound 8

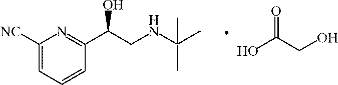

### Form A of Compound 8

Form A of compound 8 was prepared as follows:

Procedure A: Compound 1 (20.1 mg) was dissolved in EtOAc (20 V) at rt. Glycolic acid 1.05eq) was added. The resulting slurry was stirred at rt for 4 hours, and the solid was collected by filtration and dried under vacuum to yield Compound 8 Form A.

Procedure B: Compound 1 (20.0 mg) was dissolved in EtOAc (30 V) at 50° C. Glycolic acid (1.05 eq) was added. The solution was stirred at 50° C. for 1 day, during which a solid precipitated. The solid was collected by filtration and dried under vacuum to yield Compound 8 Form A.

Characterization of the resulting material demonstrated anhydrous crystalline Form A of Compound 8, with no residual organic solvent observed by NMR. The ratio of glycolate to compound 1 in Form A was determined to be 1:1 based on ¹H NMR analysis.

Table 9, supra, is reproduced below and sets forth the X-ray diffraction peaks observed for Form A of compound 8.

TABLE 9

XRPD Peak Positions for Form A of Compound 8

| Position (°2θ) | Intensity % | Position (°2θ) | Intensity % |
|---|---|---|---|
| 3.7 | 25.0 | 24.3 | 19.6 |
| 5.7 | 55.2 | 25.1 | 13.3 |
| 6.4 | 11.3 | 25.5 | 13.9 |
| 10.1 | 14.1 | 25.8 | 19.3 |
| 10.9 | 19.9 | 26.3 | 5.9 |
| 11.6 | 68.6 | 27.2 | 7.1 |
| 12.0 | 20.3 | 27.6 | 5.7 |
| 15.4 | 12.9 | 29.6 | 13.1 |
| 15.7 | 100.0 | 31.4 | 5.3 |
| 16.7 | 12.5 | 31.8 | 6.0 |
| 17.5 | 68.4 | 32.6 | 6.9 |
| 18.4 | 12.1 | 32.9 | 8.3 |
| 18.7 | 8.4 | 34.2 | 5.7 |
| 19.8 | 12.6 | 35.1 | 4.4 |
| 20.4 | 40.4 | 37.2 | 5.7 |
| 22.2 | 8.9 | 38.5 | 4.8 |
| 22.7 | 8.0 | 39.5 | 7.8 |
| 23.1 | 70.5 | | |

In this and all subsequent tables, the position (°2θ) is within ± 0.2.

FIG. 8A.1 depicts an XRPD pattern of Form A of compound 8.

FIG. 8A.2 depicts a DSC thermogram and TGA trace of Form A of compound 8. The TGA trace showed almost no weight loss before decomposition. The DSC thermogram of Form A of compound 8 was characterized by two endothermic peaks at about 122° C., and about 136° C.

FIG. 8A.3 depicts a ¹H NMR spectrum of Form A of compound 8.

### Example 9: Preparation of Form A of Compound 9

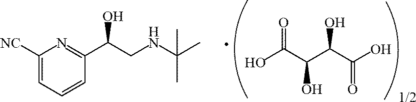

### Form A of Compound 9

Form A of compound 9 was prepared as follows:

Procedure A (salt screening method): Compound 1 (180 mg) was dissolved in MeOH (6 mL) to prepare a stock solution. Stock solution (100 µL) was added to each well of a 96 well plate. To one column of wells was added L-tartaric acid (150 µL 0.1 M solution, 1.1 eq). To each row of the plate was added a different solvent (MeOH, IPA, THF, ACN, MTBE, acetone, water and EtOAc, 200 µL each). The wells were covered with a film having a pinhole in the top and were allowed to evaporate to dryness under ambient conditions. The dried materials were collected and submitted for XRPD analysis. The wells to which L-tartaric acid was added that contained IPA, THF, ACN, acetone and EtOAc each yielded compound 9 Form A.

Procedure B: Compound 1 (~20 mg) was dissolved in EtOAc (22 V) at rt. L-tartaric acid (1 M solution in MeOH, 1.05 eq) was added to the solution and the solution was stirred at rt for 3 hours, during which a solid precipitated immediately upon addition of the acid solution. The solid was collected by filtration and dried under vacuum at 50° C. to yield Compound 9 Form A.

Procedure C: Compound 1 (~20 mg) was dissolved in acetone (25 V) at rt. L-tartaric acid (solid, 1.05 eq) was added to the solution and the solution was stirred at rt for 3 hours, during which a solid precipitated shortly after addition of the solid L-tartaric acid. The resulting precipitate was collected by filtration and dried under vacuum at 50° C. to yield Compound 9 Form A.

Procedure D: Compound 1 (121.0 mg) was dissolved in EtOAc (20 V) at rt. 1 M L-tartaric acid solution in MeOH (303.5 µL, 0.55 eq) was added to the solution and solid precipitated shortly after addition of the solid L-tartaric acid. The suspension was stirred at rt for 3 h, after which the resulting precipitate was collected by filtration and dried under vacuum at 50° C. to yield Compound 9 Form A (134.5 mg, 82.8% yield).

Characterization of the resulting material demonstrated anhydrous crystalline Form A of Compound 9, with no residual organic solvent observed by NMR. The ratio of L-tartrate to compound 1 in compound 9 Form A was determined to be 0.5:1 based on ¹H NMR analysis.

Table 10, supra, is reproduced below and sets forth the X-ray diffraction peaks observed for Form A of compound 9.

TABLE 10

XRPD Peak Positions for Form A of Compound 9

| Position (°2θ) | Intens ity % | Position (°2θ) | Intens ity % |
|---|---|---|---|
| 3.7 | 16.8 | 20.0 | 7.1 |
| 5.6 | 12.4 | 20.7 | 4.6 |
| 10.9 | 14.7 | 21.2 | 3.4 |
| 11.2 | 100.0 | 22.0 | 27.7 |
| 12.4 | 6.8 | 22.5 | 21.8 |
| 13.6 | 8.8 | 23.3 | 8.4 |
| 14.2 | 5.5 | 24.5 | 11.2 |
| 16.0 | 10.3 | 27.2 | 4.5 |
| 16.8 | 6.3 | 28.5 | 2.9 |
| 17.1 | 6.7 | 32.3 | 2.7 |
| 18.4 | 10.0 | 33.5 | 4.3 |

In this and all subsequent tables, the position (°2θ) is within ± 0.2.

FIG. 9A.1 depicts an XRPD pattern of Form A of compound 9.

FIG. 9A.2 depicts a DSC thermogram and TGA trace of Form A of compound 9. The TGA trace showed almost no weight loss below 150° C. The DSC thermogram of Form A of compound 9 was characterized by an endothermic peak at about 212° C.

FIG. 9A.3 depicts a ¹H NMR spectrum of Form A of compound 9.

FIGS. 9A.4 and 9A.5 depict DVS plots of Form A of compound 9. The DVS plot showed that Form A of compound 9 was slightly hygroscopic with 1.05% at 80%RH. The XRPD pattern of the tested sample remained unchanged after DVS testing.

### Example 10: Preparation of Form A of Compound 10

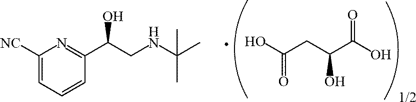

### Form A of Compound 10

Form A of compound 10 was prepared as follows:

Procedure A: Compound 1 (~20 mg) was dissolved in EtOAc (22 V) at rt. L-malic acid (1 M solution in MeOH, 1.05 eq) was added to the solution and the solution was stirred at rt for 3 hours, during which a solid precipitated shortly after addition of the acid solution. The solid was collected by filtration and dried under vacuum at 50° C. to yield Compound 10 Form A.

Characterization of the resulting material demonstrated crystalline Form A of Compound 10 as a solvate/hydrate form having small amounts of water, and about 1% residual MeOH, as observed by NMR. The ratio of L-malate to compound 1 in compound 10 Form A was determined to be 0.5:1 based on ¹H NMR analysis.

Table 11, supra, is reproduced below and sets forth the X-ray diffraction peaks observed for Form A of compound 10.

TABLE 11

XRPD Peak Positions for Form A of Compound 10

| Position (°2θ) | Intensity % | Position (°2θ) | Intensity % |
|---|---|---|---|
| 5.7 | 44.9 | 21.6 | 29.5 |
| 10.3 | 100.0 | 22.0 | 16.4 |
| 10.8 | 41.3 | 22.4 | 34.9 |
| 11.1 | 16.9 | 23.1 | 22.5 |
| 11.5 | 36.4 | 23.7 | 75.8 |
| 11.7 | 63.5 | 24.6 | 17.5 |
| 12.7 | 13.8 | 25.3 | 53.5 |
| 14.4 | 39.3 | 26.6 | 35.6 |
| 15.0 | 47.3 | 27.9 | 9.6 |
| 16.5 | 64.4 | 29.2 | 10.7 |
| 17.5 | 15.3 | 29.9 | 9.8 |
| 18.8 | 13.5 | 31.5 | 12.4 |
| 19.6 | 39.8 | 32.1 | 11.1 |
| 20.3 | 21.6 | 32.9 | 15.3 |
| 21.3 | 32.9 | | |

In this and all subsequent tables, the position (°2θ) is within ± 0.2.

FIG. 10A.1 depicts an XRPD pattern of Form A of compound 10.

FIG. 10A.2 depicts a DSC thermogram and TGA trace of Form A of compound 10. The TGA trace showed two weight loss features below 155° C.: a 1.0% weight loss between rt and 70° C. and a 1.8% weight loss between 70° C. and 125° C. Without intending to be limited to any particular theory, these weight losses are likely attributable to loss of the MeOH and water that were found to be part of Form A. The DSC thermogram of Form A of compound 10 was characterized by endothermic peaks at about 53° C., 107° C. and 155° C.

FIG. 10A.3 depicts a ¹H NMR spectrum of Form A of compound 10.

### Example 11: Preparation of Form A of Compound 11

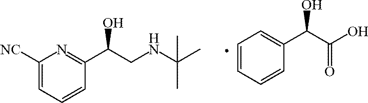

### Form A of Compound 11

Form A of compound 11 was prepared as follows:

Procedure A (salt screening method): Compound 1 (180 mg) was dissolved in MeOH (6 mL) to prepare a stock solution. Stock solution (100 µL) was added to each well of a 96 well plate. To one column of wells was added D-mandelic acid (150 µL 0.1 M solution, 1.1 eq). To each row of the plate was added a different solvent (MeOH, IPA, THF, ACN, MTBE, acetone, water and EtOAc, 200 µL each). The wells were covered with a film having a pinhole in the top and were allowed to evaporate to dryness under ambient conditions. The dried materials were collected and submitted for XRPD analysis. The wells to which D-mandelic acid was added that contained THF, MTBE and EtOAc each yielded compound 11 Form A.

Procedure B: Compound 1 (~20 mg) was dissolved in Acetone (25 V) at rt. D-mandelic acid (1.05 eq) was added to the solution and the solution was stirred at rt for 3 hours. No solid appeared after stirring. Solvent was evaporated under N₂ flow and the dried material was dissolved into EtOAc (25 V) and stirred at rt for an additional 3 hours, during which a solid precipitated. The solid was collected by filtration and dried under vacuum at 50° C. to yield Compound 11 Form A.

Procedure C: Compound 1 (~20 mg) was dissolved in EtOAc (20 V) at rt. D-mandelic acid (1.05 eq) was added to the solution and the solution was stirred at rt for 3 hours, during which a solid precipitated shortly after addition of the acid. Heptane (40 V) was added to increase the yield of precipitant and the mixture was stirred at rt for an additional 3 hours. The solid was collected by filtration and dried under vacuum at 50° C. to yield Compound 11 Form A.

Characterization of the resulting material demonstrated an anhydrous crystalline Form A of Compound 11, with no residual organic solvent, as observed by ¹H NMR. The ratio of D-mandelate to compound 1 in compound 11 Form A was determined to be 1:1 based on ¹H NMR analysis.

Table 12, supra, is reproduced below and sets forth the X-ray diffraction peaks observed for Form A of compound 11.

TABLE 12

XRPD Peak Positions for Form A of Compound 11

| Position (°2θ) | Intensity % | Position (°2θ) | Intensity % |
|---|---|---|---|
| 6.5 | 69.6 | 23.7 | 2.0 |
| 7.4 | 8.9 | 26.3 | 22.3 |
| 9.0 | 7.1 | 27.0 | 2.9 |
| 13.1 | 13.9 | 27.4 | 17.0 |
| 14.0 | 34.9 | 28.3 | 6.8 |
| 14.8 | 3.0 | 31.1 | 2.4 |
| 15.2 | 4.6 | 31.9 | 2.3 |
| 16.1 | 3.9 | 32.9 | 2.0 |
| 17.2 | 2.9 | 34.3 | 8.1 |
| 19.8 | 8.9 | 37.2 | 1.9 |
| 20.0 | 5.4 | 39.7 | 3.4 |
| 22.8 | 100.0 | | |

In this and all subsequent tables, the position (°2θ) is within ± 0.2.

FIG. 11A.1 depicts an XRPD pattern of Form A of compound 11.

FIG. 11A.2 depicts a DSC thermogram and TGA trace of Form A of compound 11. The TGA trace showed no weight loss below 100° C. The DSC thermogram of Form A of compound 11 was characterized by an endothermic peak at about 142° C.

FIG. 11A.3 depicts a ¹H NMR spectrum of Form A of compound 11.

### Example 12: Preparation of Form A of Compound 12

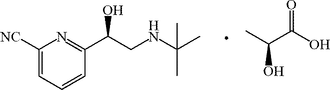

### Form A of Compound 12

Form A of compound 12 was prepared as follows:

Procedure A (salt screening method): Compound 1 (180 mg) was dissolved in MeOH (6 mL) to prepare a stock solution. Stock solution (100 µL) was added to each well of a 96 well plate. To one column of wells was added L-lactic acid (150 µL 0.1 M solution, 1.1 eq). To each row of the plate was added a different solvent (MeOH, IPA, THF, ACN, MTBE, acetone, water and EtOAc, 200 µL each). The wells were covered with a film having a pinhole in the top and were allowed to evaporate to dryness under ambient conditions. The dried materials were collected and submitted for XRPD analysis. The wells to which L-lactic acid was added that contained MeOH, THF, MTBE and acetone each yielded compound 12 Form A.

Procedure B: Compound 1 (~20 mg) was dissolved in acetone (25 V) at rt. L-lactic acid (1.05 eq) was added to the solution and the solution was stirred at rt for 3 hours. No solid appeared after stirring. Solvent was evaporated under N₂ flow and the dried material was dissolved into EtOAc (25 V) and stirred at rt for an additional 3 hours. Heptane (80 V) was added and the mixture was stirred at rt for 1 day, during which a solid precipitated. The solid was collected by filtration and dried under vacuum at 50° C. to yield Compound 12 Form A.

Characterization of the resulting material from Procedure B demonstrated a form with low crystallinity, which was assigned as crystalline Form A of Compound 12. The materials resulting from Procedure A demonstrated greater crystallinity.

Table 13, supra, is reproduced below and sets forth the X-ray diffraction peaks observed for Form A of compound 12.

TABLE 13

XRPD Peak Positions for Form A of Compound 12

| Position (°2θ) | Intensity % | Position (°2θ) | Intensity % |
|---|---|---|---|
| 5.0 | 58.5 | 22.1 | 59.5 |
| 6.4 | 18.5 | 22.8 | 27.9 |
| 7.1 | 15.0 | 23.2 | 19.1 |
| 10.1 | 100.0 | 24.0 | 15.8 |
| 10.4 | 77.7 | 24.6 | 29.6 |
| 10.8 | 47.7 | 25.0 | 19.3 |
| 11.1 | 56.6 | 25.6 | 29.6 |
| 13.7 | 25.1 | 25.8 | 19.4 |
| 15.2 | 28.3 | 26.4 | 20.1 |
| 15.7 | 23.2 | 27.7 | 22.3 |
| 16.2 | 53.1 | 28.6 | 16.9 |
| 17.0 | 24.1 | 29.1 | 13.4 |
| 17.4 | 26.3 | 30.2 | 9.9 |
| 17.7 | 27.9 | 31.0 | 13.9 |
| 19.1 | 16.0 | 31.3 | 13.4 |
| 19.8 | 24.0 | 31.9 | 12.4 |
| 20.4 | 29.2 | 32.6 | 12.5 |
| 21.3 | 65.0 | 35.8 | 11.6 |

In this and all subsequent tables, the position (°2θ) is within ± 0.2.

FIG. 12A.1 depicts an XRPD pattern of Form A of compound 12.

FIG. 12A.2 depicts a DSC thermogram and TGA trace of Form A of compound 12. The TGA trace showed weight loss of about 2.9% before 100° C. The DSC thermogram of Form A of compound 12 was complex and showed multiple overlapping endothermic peaks between rt and 120° C., with prominent endothermic features at about 39° C., 76° C. and 95° C.

### Example 13: Preparation of Forms A and B of Compound 13

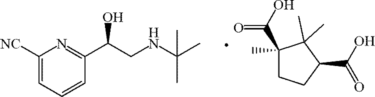

### Form A of Compound 13

Form A of compound 13 was prepared as follows:

Procedure A: Compound 1 (20 mg) was dissolved in EtOAc (20 V) at rt. D-camphoric acid (1.05 eq) was added to the solution and the solution was stirred at rt for 2 hours. No solid appeared after stirring. Heptane (0.8 mL) was added and the mixture was stirred at rt for an additional 2 hours, during which a solid precipitated. The solid was collected by filtration and dried under vacuum at 50° C. to yield Compound 13 Form A.

Characterization of the resulting material demonstrated an anhydrous crystalline Form A of Compound 13, with no residual organic solvent, as observed by ¹H NMR. The ratio of D-camphorate to compound 1 in compound 13 Form A was determined to be 1:1 based on ¹H NMR analysis.

Table 14, supra, is reproduced below and sets forth the X-ray diffraction peaks observed for Form A of compound 13.

TABLE 14

XRPD Peak Positions for Form A of Compound 13

| Position (°2θ) | Intensity % | Position (°2θ) | Intensity % |
|---|---|---|---|
| 4.5 | 10.9 | 22.2 | 3.2 |
| 7.9 | 100.0 | 23.2 | 4.0 |
| 8.6 | 10.3 | 23.6 | 3.4 |
| 9.5 | 35.3 | 24.9 | 3.6 |
| 10.1 | 7.6 | 25.6 | 3.7 |
| 10.9 | 3.6 | 26.1 | 6.1 |
| 11.7 | 23.7 | 26.5 | 4.7 |
| 12.9 | 19.4 | 27.9 | 2.8 |
| 13.3 | 2.5 | 28.6 | 2.8 |
| 14.7 | 20.4 | 29.1 | 2.6 |
| 15.7 | 3.6 | 29.4 | 3.9 |
| 16.2 | 10.0 | 29.7 | 5.9 |
| 16.8 | 13.1 | 31.0 | 5.3 |
| 17.2 | 21.4 | 32.0 | 1.9 |
| 17.8 | 4.9 | 32.9 | 2.3 |
| 18.1 | 11.3 | 33.1 | 3.4 |
| 18.5 | 20.5 | 34.8 | 1.9 |
| 18.9 | 21.6 | 36.5 | 2.0 |
| 19.1 | 13.4 | 37.4 | 2.8 |
| 20.3 | 3.8 | 37.7 | 2.2 |
| 20.6 | 3.9 | 38.8 | 2.2 |
| 21.1 | 7.5 | 39.6 | 2.1 |
| 21.8 | 7.2 | | |

In this and all subsequent tables, the position (°2θ) is within ± 0.2.

FIG. 13A.1 depicts an XRPD pattern of Form A of compound 13.

FIG. 13A.2 depicts a DSC thermogram and TGA trace of Form A of compound 13. The TGA trace showed no weight loss below 100° C. The DSC thermogram of Form A of compound 13 was characterized by a minor endothermic peak at about 147° C. and a larger endothermic peak at about 166° C.

FIG. 13A.3 depicts a ¹H NMR spectrum of Form A of compound 13.

### Example 14: Preparation of Form A of Compound 14

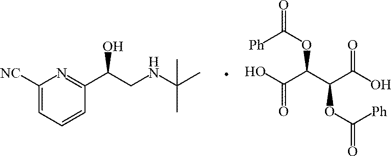

### Form A of Compound 14

Form A of compound 14 was prepared as follows:

Procedure A: Compound 1 (~20 mg) was dissolved in IPA (0.6 mL) at rt. Dibenzoyl-D-tartaric acid (1.05 eq) was added to the solution and the solution was stirred at rt for 2 hours, during which solid began precipitating shortly after adding the acid. The solid was collected by filtration and dried under vacuum at 50° C. to yield Compound 14 Form A.

Characterization of the resulting material demonstrated an anhydrous crystalline Form A of Compound 14, with no residual organic solvent, as observed by NMR. The ratio of dibenzoyl-D-tatrate to compound 1 in compound 14 Form A was determined to be 1:1 based on ¹H NMR analysis.

Table 15, supra, is reproduced below and sets forth the X-ray diffraction peaks observed for Form A of compound 14.

TABLE 15

XRPD Peak Positions for Form A of Compound 14

| Position (°2θ) | Intensity % | Position (°2θ) | Intensity % |
|---|---|---|---|
| 4.9 | 8.6 | 19.6 | 5.6 |
| 6.3 | 100.0 | 20.3 | 2.9 |
| 6.8 | 9.6 | 20.7 | 5.6 |
| 8.8 | 36.1 | 21.1 | 2.8 |
| 11.2 | 7.7 | 22.3 | 4.6 |
| 12.2 | 26.9 | 22.6 | 5.7 |
| 12.6 | 34.7 | 23.0 | 7.5 |
| 12.8 | 25.3 | 23.4 | 7.9 |
| 14.0 | 18.0 | 23.8 | 8.8 |
| 14.2 | 12.8 | 24.3 | 10.5 |
| 14.8 | 4.3 | 24.6 | 4.4 |
| 15.3 | 4.4 | 25.1 | 6.9 |
| 15.6 | 9.0 | 25.3 | 5.4 |
| 16.2 | 4.9 | 25.9 | 4.3 |
| 16.5 | 7.3 | 26.2 | 4.7 |
| 16.9 | 18.1 | 26.5 | 5.0 |
| 17.2 | 13.8 | 27.2 | 5.1 |
| 17.6 | 7.7 | 27.8 | 3.2 |
| 18.2 | 7.7 | 28.2 | 2.5 |
| 18.9 | 13.7 | 32.5 | 2.4 |
| 19.3 | 11.7 | | |

In this and all subsequent tables, the position (°2θ) is within ± 0.2.

FIG. 14A.1 depicts an XRPD pattern of Form A of compound 14.

FIG. 14A.2 depicts a DSC thermogram and TGA trace of Form A of compound 14. The TGA trace showed no weight loss below 150° C. The DSC thermogram of Form A of compound 14 was characterized by an endothermic peak at about 182° C.

FIG. 14A.3 depicts a ¹H NMR spectrum of Form A of compound 14.

### Example 15: Chiral Purification of Compound 1 Through Formation of Chiral Acid Salts

Several crystalline salt forming acids described above were tested for their ability to enhance the chiral purity of a mixture of compound 1 and its enantiomer. Compound 1, (S)-6-(2-(tert-butylamino)-1-hydroxyethyl)picolinonitrile), and its enantiomer, (R)-6-(2-(tert-butylamino)-1-hydroxyethyl)picolinonitrile were mixed together in an 80/20 or 90/10 ratio, dissolved in a solvent at rt or 60° C., and one of the acid forming salts was added to the solution, as described in Table 17 below:

TABLE 17

Chiral Purification Evaluation

| Entry | Initial chiral purity | Solvent | Acid | Cmpd | Crystallization Method | Final chiral purity |
|---|---|---|---|---|---|---|
| 1 | 80% | EtOAc (20V) | 1 M L-tartaric acid in MeOH | 9 | Precip. occurred quickly after adding the acid, stirred at rt for 3 h | 87.14% |
| 2 | 80% | EtOH (20V) | Solid L-tartaric acid | 9 | Precip. occurred quickly after adding the acid, stirred at rt for 2 h | 88.20% |
| 3 | 80% | EtOH (20V) | 1 M L-tartaric acid in MeOH | 9 | Precip. occurred ~2 min after adding the acid at 60° C., stirred at 60° C. for 2 h and rt for 1 h | 89.89% |
| 4* | 80% | EtOH (30V) | 1 M L-tartaric acid in MeOH | 9 | Precip. occurred quickly after adding the acid at 60° C., stirred at 60° C. for 2 h | 86.35% |
| 5* | 80% | THF (20V) | 1 M L-tartaric acid in MeOH | 9 | Precip. occurred quickly after adding the acid at 60° C., stirred at 60° C. for 2 h | 85.15% |
| 6* | 80% | ACN (20V) | 1 M L-tartaric acid in MeOH | 9 | Precip. occurred quickly after adding the acid at 60° C., stirred at 60° C. for 2 h | 86.96% |
| 7* | 80% | DCM (20V) | 1 M L-tartaric acid in MeOH | 9 | Precip. occurred quickly after adding the acid at rt, stirred at rt for 2 h | 89.37% |
| 8* | 90% | EtOH (30V) | 1 M L-tartaric acid in MeOH | 9 | Precip. occurred quickly after adding the acid at 60° C., stirred at 60° C. for 2 h | 94.64% |
| 9* | 90% | DCM (30V) | 1 M L-tartaric acid in MeOH | 9 | Precip. occurred ~10 min after adding the acid at rt, stirred at rt for 2 h | 95.72% |
| 10 | 80% | EtOAc (20V) | 1 M L-malic acid in MeOH | 10 | Trace solid precip. after stirring at rt for 1 h, additional 15 V EtOAc was added, stirred for additional 1 h | 95.12% |
| 11 | 80% | EtOAc (20V) | 1 M L-malic acid in MeOH | 10 | No precip. after stirring 2h at 60° C., additional 18 V EtOAc was added, stirred at rt for 1 d, precip. occurred >2 h at rt | 95.92% |
| 12* | 80% | EtOAc (20V) | 1 M L-malic acid in MeOH | 10 | No precip. after stirring 2h at 60° C., additional 100 V EtOAc was added, stirred at rt for 1 d | Sticky |
| 13* | 80% | IPA (30V) | 1 M L-malic acid in MeOH | 10 | No precip. after stirring 2 h at 60° C., 150 V EtOAc was added, stirred at rt for 1 d | Sticky |
| 14* | 80% | MEK (20V) | 1 M L-malic acid in MeOH | 10 | No precip. after stirring 1h at 60° C., 250 V MTBE was added, stirred at rt for 1 d | Solution |
| 15* | 80% | iPrOAc (20V) | 1 M L-malic acid in MeOH | 10 | No precip. after stirring 1h at 60° C., precip. occurred after stirring at rt for 2 h | Sticky |
| 16* | 80% | iPrOAc (20V) | Solid L-malic acid | 10 | Oil obtained after stirring at 60° C. for 1 h, added 100 V heptane and stirred at 60° C. for 1 d | 80.33% |
| 17* | 80% | 2-Me-2-butanol (30V) | Solid L-malic acid | 10 | Precip. occurred quickly after adding the acid, but little amount after being stirred at 50° C. for 1 h. 50 V MTBE added and stirred at 50° C. for another 2 h | 83.24% High Yield |
| 18 | 80% | EtOAc (20V) | Solid D-mandelic acid | 11 | Precip. occurred ~10 min after adding the acid at rt, stirred at rt for 2 h | 92.19% |
| 19 | 80% | EtOAc (20V) | Solid D-mandelic acid | 11 | Precip. occurred 2 h after adding the acid at 60° C., cooled to rt and stirred for another 1 h | 88.12% |
| 20 | 80% | MEK (25V) | Solid D-camphoric acid | 13 | No precip. after stirring 1 h at 60° C. for 1 h and rt for 2 h, 100 V MTBE added and stirred at rt for 1d | Solution |
| 21 | 80% | iPrOAc (25V) | Solid D-camphoric acid | 13 | Precip. occurred quickly after adding the acid at 60° C., stirred at 60° C. for 2 h | 59.71% |

Note: Salts denoted with * were formed using 0.55 eq acid and others were formed using 1.05 eq acid

Chiral purity was determined via HPLC analysis. The results of the chiral purification screen indicated that addition of L-tartaric acid substantially reduced the content of (R)-6-(2-(tert-butylamino)-l-hydroxyethyl)picolinonitrile in the final collected solids. Under certain conditions, addition of L-tartaric acid was able to reduce the content of the undesired enantiomer by more than half.

### Example 16: Solubility and Stability Testing

### Solubility Testing

Compound 1 Form A, Compound 2 Form A and Compound 9 Form A were tested for stability in three biorelevant media and water at 37° C. for 0.5, 2 and 24 hours. About 15 mg of Compound 1 Form A, Compound 2 Form A and Compound 9 Form A were weighted into four separate vials each and to each set of vials was added 3 mL of one of the following biorelevant media: simulated gastric and intestinal fluid (SGF), fasted state simulated intestinal fluid (FaSSIF), fed state simulated intestinal fluid (FeSSIF), and water. All samples were shaken at 200 rpm at 37° C. for up to 24 hours. Compound 2 Form A dissolved immediately at rt in each of the 4 media. Compound 9 Form A dissolved in each medium after about 15 minutes of shaking at 37° C. Compound 1 Form A dissolved in each medium over the course of about 2 hours of shaking at 37° C. Compound 1 Form A, Compound 2 Form A and Compound 9 Form A all showed high solubility in each medium (> 5 mg/ml). The results of the solubility test are summarized below in Table 18:

TABLE 18

Solubility Evaluation Results

| Compound | Medium | Solubility (mg/mL) | | pH |
|---|---|---|---|---|
| | | 0.5 h | 2 and 24 h | 24 h |
| Compound 1 Form A | SGF | 4.76 | >5 | 1.20 |
| | FeSSIF | 4.41 | >5 | 5.52 |
| | FaSSIF | 4.33 | >5 | 8.03 |
| | Water | 4.19 | >5 | 10.35 |
| Compound 2 Form A | SGF | >5 | >5 | 1.11 |
| | FeSSIF | >5 | >5 | 5.01 |
| | FaSSIF | >5 | >5 | 6.47 |
| | Water | >5 | >5 | 5.24 |
| Compound 9 Form A | SGF | >5 | >5 | 1.18 |
| | FeSSIF | >5 | >5 | 5.01 |
| | FaSSIF | >5 | >5 | 6.51 |
| | Water | >5 | >5 | 5.68 |

Note: pH of water, SGF, FaSSIF and FeSSIF controls were 5.26, 1.19, 6.53, and 5.01 respectively

As all of the samples dissolved after about 2 hours, estimated solubility testing was conducted, wherein about 5 mg of Compound 1 Form A, Compound 2 Form A and Compound 9 Form A were weighed into four separate vials each and each media was added stepwise while stirring at rt until the solid dissolved. The results of the estimated solubility test are summarized in Table 19:

TABLE 19

Estimated Solubility Results

| Compound | Solubility (mg/mL) | | | |
|---|---|---|---|---|
| | SGF | FeSSIF | FaSSIF | Water |
| Compound 1 Form A | ~12.7 | ~9.1 | ~6.3 | ~5.0 |
| Compound 2 Form A | >101.2 | >100.4 | >100 | >101.6 |
| Compound 9 Form A | >71.4 | >101.4 | >71.4 | >71.3 |

It was found that Compound 2 Form A and Compound 9 Form A were much more soluble than the free base Compound 1 Form A, and the HC1 salt Compound 2 had the highest solubility in all media.

### Stability Testing

Solid stability of Compound 1 Form A, Compound 2 Form A and Compound 9 Form A were evaluated at 60° C. and 40° C./75%RH for 7 days. Purity was determined via HPLC. The results are summarized below in Table 20. Compound 2 and Compound 9 remained highly stable over the 7-day experiment with the purity of the Compound 2 HC1 salt remaining nearly unchanged. The free base showed a decrease in purity of ~0.3% under both sets of conditions. All tested compounds retained the same solid form after the 7-day experiment.

TABLE 20

Stability Evaluation Results

| Compound | Purity @ 0 d (Area%) | Purity @ 7 d (Area%) | | Solid form @ 7 d | |
|---|---|---|---|---|---|
| | | 40° C./75%RH | 60° C. | 40° C./75%RH | 60° C. |
| Compound 1 Form A | 96.09% | 95.78% | 95.76% | unchanged | unchanged |
| Compound 2 Form A | 99.27% | 99.27% | 99.25% | unchanged | unchanged |
| Compound 9 Form A | 98.62% | 98.49% | 98.62% | unchanged | unchanged |

1. A crystalline solid form of Compound 1: 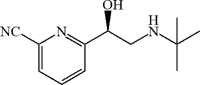 [SMILES: CC(C)(C)NC[C@H](O)c1cccc(C#N)n1]
selected from Form A and Form B.

2. The crystalline solid form according to claim 0, wherein said compound is a crystalline solid substantially free of amorphous compound 1.

3-207. (canceled)

208. The crystalline solid form according to claim 1, having one or more peaks in its X-ray powder diffraction pattern selected from those at about 9.3, about 12.6, about 16.9, about 18.8, about 20.6, and about 25.1 degrees 2-theta.

209. The crystalline solid form according to claim 1, having a DSC thermogram characterized by endothermic peaks at about 100° C., and about 104° C.

210. The crystalline solid form according to claim 1, having an X-ray powder diffraction pattern substantially as shown in Figure 1A.1.

211. The crystalline solid form according to claim 1, having a DSC thermogram substantially as shown in Figure 1A.2.

212. A salt form of compound 1: 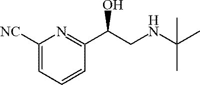 [SMILES: CC(C)(C)NC[C@H](O)c1cccc(C#N)n1]
selected from: 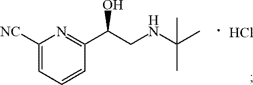 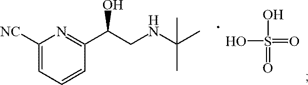 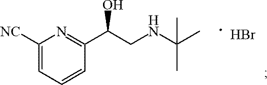 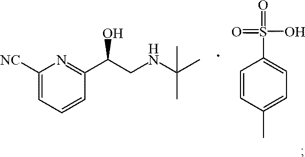 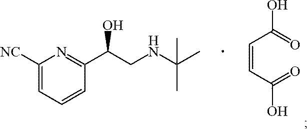 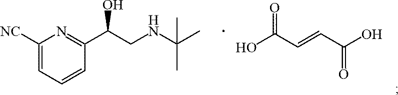 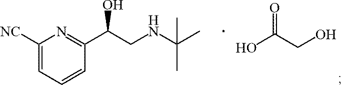 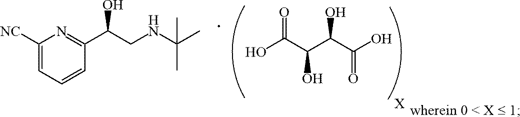 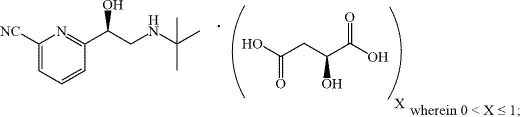 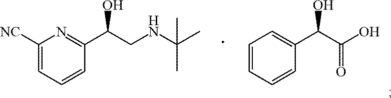 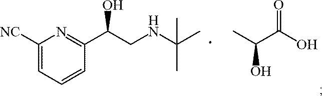 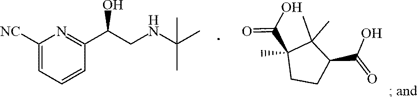 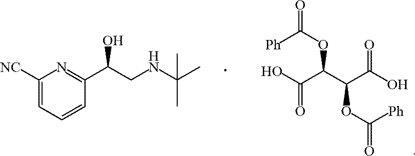
.

213. The salt form of claim 212, wherein the salt form of Compound 1 is Compound 2:

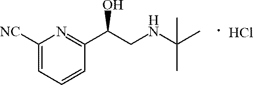

214. The salt form according to claim 213, wherein said salt form is crystalline.

215. The salt form according to claim 213, wherein said salt form is a crystalline solid substantially free of amorphous compound 2.

216. The salt form of claim 212 wherein the salt form of Compound 1 is Compound 3:

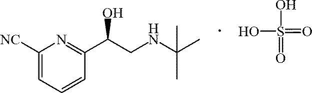 [SMILES: CC(C)(C)NC[C@H](O)c1cccc(C#N)n1 ; O=S(=O)(O)O]

217. The salt form according to claim 216, wherein said salt form is crystalline.

218. The salt form according to claim 217, wherein said salt form is a crystalline solid substantially free of amorphous compound 3.

219. A composition comprising a crystalline solid form or salt form according to claim 1 and a pharmaceutically acceptable carrier or excipient.

220. A composition comprising a crystalline solid form or salt form according to claim 212 and a pharmaceutically acceptable carrier or excipient.

221. A method of modulating the activity of one or both of β1-adrenergic receptor and β2-adrenertic receptor in a patient, comprising administering to said patient a crystalline solid form or salt form according to claim 1, or a composition thereof.

222. A method of modulating the activity of one or both of β1-adrenergic receptor and β2-adrenertic receptor in a patient, comprising administering to said patient a crystalline solid form or salt form according to claim 212, or a composition thereof.

223. A method of treating a β1-adrenergic receptor or β2-adrenergic receptor mediated disease or disorder in a patient, comprising administering to said patient a crystalline solid form or salt form according to claim 1, or a composition thereof.

224. A method of treating a β1-adrenergic receptor or β2-adrenergic receptor mediated disease or disorder in a patient, comprising administering to said patient a crystalline solid form or salt form according to claim 212, or a composition thereof.

225. The method according to claim 0, wherein the β1-adrenergic receptor or β2-adrenergic receptor mediated disease or disorder is one or more selected from the group consisting of MCI (mild cognitive impairment), aMCI (amnestic MCI), Vascular Dementia, Mixed Dementia, FTD (fronto-temporal dementia; Pick’s disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wemicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson’s disease), PDD (PD dementia), ADHD (attention deficit hyperactivity disorder), Alzheimer’s disease (AD), early AD, and Down Syndrome (DS).
